(19) United States
(12) Patent Application Publication
(10) Pub. No.: US 2024/0327339 A1
(43) Pub. Date: Oct. 3, 2024

(54) COMPOSITIONS AND METHODS FOR TREATING AND PREVENTING VIRAL INFECTIONS

(71) Applicant: University of Georgia Research Foundation, Inc., Athens, GA (US)

(72) Inventors: Ralph A. Tripp, Wakinsville, GA (US); Jackelyn Murray, Monroe, GA (US); Devi Kalyan Karumanchi, Chicago, IL (US); David Eugene Martin, Shawnee, OK (US)

(21) Appl. No.: 18/577,631

(22) PCT Filed: Jul. 6, 2022
(86) PCT No.: PCT/US2022/073476

§ 371 Date: Jan. 8, 2024

Related U.S. Application Data
(60) Provisional application No. 63/203,026, filed on Jul. 6, 2021.
(60) Provisional application No. 63/349,364, filed on Jun. 6, 2022.

(51) Int. Cl. C07C 311/16; A23K 20/111; A23K 50/30; A23K 50/75; A61K 31/18; A61P 31/14

(52) U.S. Cl. CPC C07C 311/16; A23K 20/111; A23K 50/30; A23K 50/75; A61K 31/18; A61P 31/14

(57) ABSTRACT

Probenecid prodrug compounds, and formulations including one or more of probenecid, metabolites and analogs thereof, and prodrugs thereof, and pharmaceutically acceptable salt thereof are provided. Delivery vehicles and pharmaceutical compositions including any of the foregoing are also provided. Methods of using the compounds and compositions to treat viral infections are also provided. In some embodiments, the viral infection is caused by an RNA virus. Particular viruses include, but are not limited to, influenza virus A, influenza virus B, or influenza virus C, respiratory syncytial virus (RSV), coronaviruses, measles virus, mumps virus, Zika virus, and dengue virus. Dosage regimens are also provided.

### CROSS-REFERENCE TO RELATED APPLICATIONS

This application claims the benefit of and priority to U.S. Ser. No. 63/203,026, filed on Jul. 6, 2021, and U.S. Ser. No. 63/349,364, filed on Jun. 6, 2022, each of which is incorporated by reference herein in its entirety.

### REFERENCE TO SEQUENCE LISTING

The Sequence Listing submitted as an xml file named “UGA2022-128-03PCT.xml,” created on Jul. 6, 2022, and having a size of 146,686 bytes is hereby incorporated by reference pursuant to 37 C.F.R. § 1.834(c)(1).

### FIELD OF THE INVENTION

The field of invention generally relates to small molecule compounds and formulations thereof, and methods of use thereof for the treatment of viral infections.

### BACKGROUND OF THE INVENTION

Viral replication and transmissibility are the principal causes of endemic and pandemic disease threats. There remains a need for broad-spectrum antiviral agents. Examples of most common respiratory viruses are endemic agents such as coronaviruses, respiratory syncytial viruses, and influenza viruses. Although vaccines are available for severe acute respiratory syndrome coronavirus 2 (SARS-CoV-2) and some influenza viruses, there is a paucity of effective antiviral drugs, while for RSV there is no vaccine available, and therapeutic treatments are very limited.

For example, there are currently only two FDA-approved drugs for RSV: palivizumab, a monoclonal antibody for the prevention of RSV in high-risk children, and ribavirin, approved for the treatment of severe RSV disease. Both of these drugs have questionable effectiveness (Bergeron, et al., Expert Opin Investig Drugs, 29, 285-294, doi:10.1080/13543784.2020.1735349 (2020)). Despite the availability of these approved drugs, RSV remains a worldwide health concern due to the lack of a safe and effective vaccine, and substantial morbidity and some mortality across a spectrum of ages, i.e., the young and old. Several promising antiviral candidates with different mechanisms of action (Bergeron, et al., Expert Opin Investig Drugs, 29, 285-294, doi:10.1080/13543784.2020.1735349 (2020), Boyoglu-Barnum, et al., Expert Opin Biol Ther, 20, 1073-1082, doi:10.1080/14712598.2020.1753696 (2020)) are helping to advance development (Soto, et al., Front Immunol 11, 1507, doi:10.3389/fimmu.2020.01507 (2020), Drydale, et al., Sci Transl Med 12, doi:10.1126/scitranslmed.aax2466 (2020)), however, new options for treating and preventing RSV disease and other viral infections are needed.

Probenecid has been identified as a repurposed therapeutic effective for the treatment of influenza and SARS-CoV-2. See, e.g., U.S. Published Application No. 2014/0121237 and U.S. Pat. No. 11,116,737. Probenecid is a highly lipid soluble benzoic acid derivative with an excellent safety profile that was developed in the 1950's to decrease the serum concentrations of uric acid in patients with gout and the renal tubular excretion of penicillin. Probenecid, USP is a white or nearly white, fine, crystalline powder. Probenecid is soluble in dilute alkali, in alcohol, in chloroform, and in acetone, however, it is practically insoluble in water and in dilute acids. Probenecid is completely absorbed after oral administration, where peak plasma concentration is reached in 2-4 hr. The half-life of the drug in plasma is dose dependent and varies from less than 5 hr to more than 8 hr. It is often administered in the form of high dose tablets and has less patient compliance especially in pediatric patients. Thus, there remains a need for improved, more soluble and patient compliant probenecid prodrugs and formulations.

Thus, it is an object of provide compounds, compositions, and methods of using the same for the treatment of viruses.

It is also an object of the invention to provide probenecid analogs and prodrug, preferably with improved solubility, and compositions and methods of use thereof.

### SUMMARY OF THE INVENTION

Synthetic compounds that can be used as prodrugs of probenecid or probenecid analogs have been developed. These compounds (also referred to herein as “prodrugs”) are inactive forms prepared from active probenecid or probenecid analogs, which can release probenecid or probenecid analogs in their active forms after being administered into a subject in need thereof. The released probenecid or probenecid analogs have antiviral properties and should be suitable for use in the prevention and/or treatment of multiple classes of viruses.

In some forms, the compound is formed by modifying the carboxyl group of probenecid using a water-soluble moiety, such as a carbonate, carbamate, imine, ether, ester, amide, or phosphate. The addition of such water-soluble moieties can effectively diminish the ability of these compounds to cross certain biological membranes, such as those associated with the blood-brain barrier or the blood-placental barrier. In some forms, these compounds show reduced biological membrane crossing rate as compared to the biological membrane crossing rate of probenecid not attached to the water-soluble moiety. Additionally, compared to probenecid administered in its free form, the prodrugs disclosed herein can alter the pharmacokinetics of probenecid, improve the stability and solubility of probenecid, decrease the toxicity of probenecid, increase the specificity of probenecid, and/or increase the duration of the pharmacological effect of probenecid.

The prodrug can have the structure of Formula I:

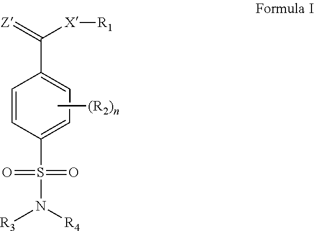

where: (a) Z′ is O, NR₅, or S; (b) X′ can be absent, O, NR₅, or S; (c) R₁ can be hydrogen, a substituted or unsubstituted alkyl, a substituted or unsubstituted alkenyl, a substituted or unsubstituted alkynyl, a substituted or unsubstituted aryl, a substituted or unsubstituted heteroaryl, a substituted or unsubstituted polyaryl, a substituted or unsubstituted polyheteroaryl, an azo, an alkoxy, a polyether, a thiol, a sulfanimine, an amino, a carbonate, an ester, an amide, a carbamate, an imine, a substituted or unsubstituted carbonyl, a hydroxyl, a polyol, a phosphonyl, sulfinyl, a sulfonamide, a nitro, a cyano, a lipid, a peptide, a cholesterol, a phytosterol, a glycoside, or a glucuronide; (d) n can be an integer from 0 to 4; (e) each R₂ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted aralkyl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen; (f) R₃-R₅ can be independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol; and (g) the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

In some forms of Formula I, R₁ can be hydrogen, a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, a substituted or unsubstituted heteropolyaryl, a polyol, a polyalkylene glycol, a lipid, a peptide, a cholesterol, a phytosterol, a glucuronide,

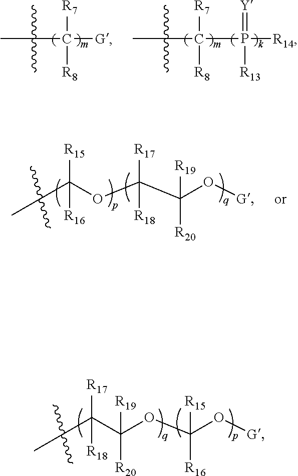

wherein G′ can be hydrogen, a lipid, a peptide, a cholesterol, a phytosterol, a glycoside, a glucuronide,

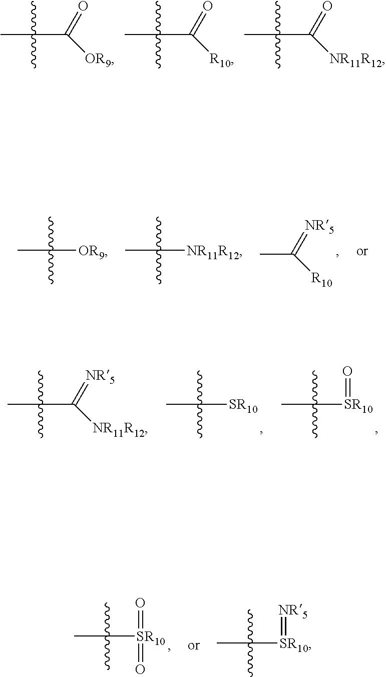

and R₉-R₁₂ can be independently hydrogen, a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, an alkoxy, a di-alkyl amino, or a halogen; R′₅ can be independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol, such as a hydrogen or a substituted or unsubstituted C₁-C₆ alkyl (e.g., an unsubstituted C₁-C₆ linear or branched alkyl, an unsubstituted C₁-C₆ cycloalkyl, an unsubstituted C₁-C₄ linear or branched alkyl, an unsubstituted C₁-C₄ cycloalkyl, an unsubstituted C₁-C₃ linear or branched alkyl, an unsubstituted C₁-C₃ cycloalkyl, etc.); m, k, p, and q can be independently an integer from 0 to 20, from 0 to 18, from 0 to 16, from 0 to 14, from 0 to 12, from 0 to 10, from 0 to 8, from 0 to 6, from 0 to 4, from 0 to 3, or from 0 to 2, such as 0 or 1; each Y′ can be independently O or S; each occurrence of R₇, R₈, and R₁₅-R₂₀ can be independently hydrogen, a substituted or unsubstituted C₁-C₂₀ alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen, or R₇ and R₈ together, R₁₅ and R₁₆ together, and/or R₁₇ and R₁₈ together, with the carbon atom to which they are attached, form a C₁-C₂₀ cycloalkyl, or when X′ is NR₅, m is not 0, at least one of p and q is not 0, then (i) R₇ is hydrogen and R₈ is a substituted or unsubstituted C₁-C₂₀ alkyl that form a ring together with R₅ that includes the adjoining N and C atoms, (ii) R₁₅ is hydrogen and R₁₆ is a substituted or unsubstituted C₁-C₂₀ alkyl that form a ring together with R₅ that includes the adjoining N and C atoms, and/or (iii) R₁₇ is hydrogen and R₁₈ is a substituted or unsubstituted C₁-C₂₀ alkyl that form a ring together with R₅ that includes the adjoining N and C atoms; and R₁₃ and R₁₄ can be independently hydrogen, a substituted or unsubstituted C₁-C₂₀ alkyl, or an alkoxy.

In some forms of Formula I, R₁ can be an unsubstituted C₁-C₂₀ linear or branched alkyl, an unsubstituted C₃-C₂₀ cycloalkyl, a C₁-C₂₀ haloalkyl, an unsubstituted aryl, an unsubstituted polyaryl, an unsubstituted heteroaryl, an unsubstituted heteropolyaryl, a polyalkylene glycol, a lipid, a peptide, a cholesterol, a phytosterol, a glucuronide,

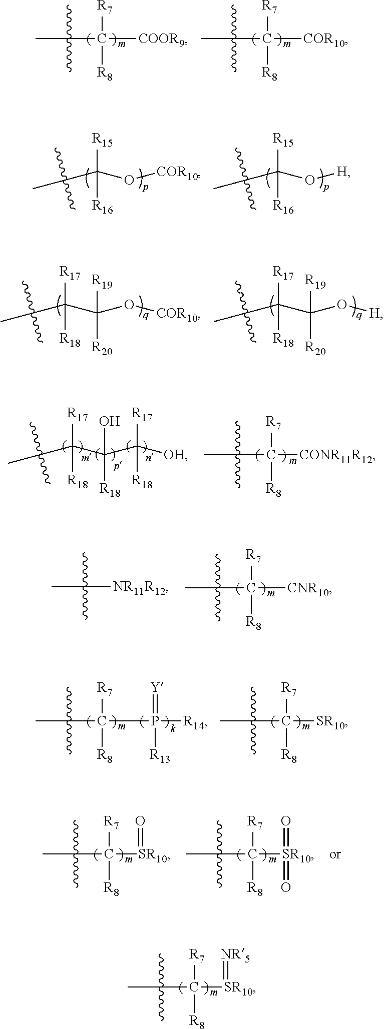

and wherein m, m′, p′, and n′ can be independently an integer from 0 to 10, from 0 to 8, from 0 to 6, from 0 to 4, from 0 to 3, from 0 to 2, or 0 or 1, p is an integer from 1 to 6, from 1 to 4, from 1 to 3, or 1 or 2, and k is an integer from 1 to 6, from 1 to 4, or.

In some forms of Formula I, Z′ can be O, X′ can be absent or O, and R₁ can be a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, a substituted or unsubstituted heteropolyaryl, a polyalkylene glycol, a lipid, a peptide, a cholesterol, a phytosterol, a glycoside, or a glucuronide.

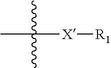

In some forms of Formula I, Z′ can be O or NR₅ and can be

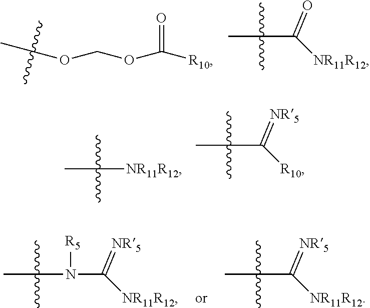

In some forms of Formula I, Z′ can be O, X′ can be O, and R₁ can be

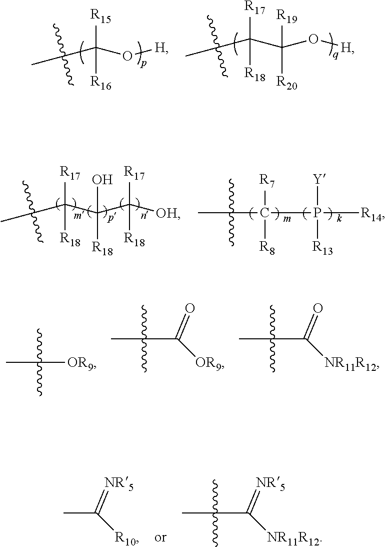

In some forms of Formula I, Z′ can be O, X′ can be O, and R₁ can be

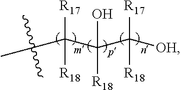

where m′ and n′ are independently an integer from 0 to 6, from 0 to 5, from 0 to 4, from 0 to 3, from 1 to 3, such as 1 or 2; p′ is an integer from 1 to 10, from 1 to 8, from 1 to 6, from 1 to 4, or from 1 to 3; each occurrence of R₁₇ and R₁₈ are independently hydrogen, hydroxyl, —SH, —(CH₂)₁₋₆NR₂₂R₂₃, —(CH₂)₁₋₆OH, —(CH₂)₁₋₆SH, or an unsubstituted C₁-C₁₀ alkyl. In some forms of Formula I, Z′ can be O, X′ can be O, and R₁ can be

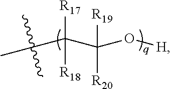

where q is an integer from 1 to 10, from 1 to 8, from 1 to 6, from 1 to 4, or from 1 to 3; each occurrence of R₁₇-R₂₀ are independently hydrogen, hydroxyl, —SH, —(CH₂)₁₋₆NR₂₂R₂₃, —(CH₂)₁₋₆OH, —(CH₂)₁₋₆SH, or an unsubstituted C₁-C₁₀ alkyl. In these forms of Formula I, R₂ can be hydrogen, hydroxyl, —SH, —(CH₂)₁₋₆NR₂₂R₂₃, —(CH₂)₁₋₆OH, —(CH₂)₁₋₆SH, or an unsubstituted C₁-C₁₀ alkyl, such as hydrogen; and R₃ and R₄ can be independently a an unsubstituted C₁-C₁₀ alkyl, such as an unsubstituted methyl, ethyl, propyl, butyl, pentyl, or hexyl, for example, an unsubstituted propyl.

In some forms of Formula I, Z′ can be O, X′ can be S, and R₁ can be

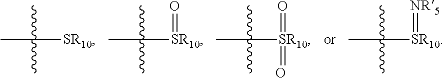

In any one of the forms of Formula I described above, each occurrence of R₇, R₈, and R₁₅-R₂₀ can be independently hydrogen, a substituted or unsubstituted C₁-C₂₀ alkyl, —(CH₂)₁₋₆NR₂₂R₂₃, —(CH₂)₁₋₆OH, a substituted or unsubstituted aralkyl, —(CH₂)₁₋₆SH, —(CH₂)₁₋₆S(O)₀₋₂CH₃, —(CH₂)₁₋₆NHC(═NH)NH₂, -(1H-indol-3-yl) methyl, -(1H-imidazol-4-yl)methyl, —(CH₂)₀₋₆COOR₂₁, —(CH₂)₀₋₆CONR₂₂R₂₃, a substituted or unsubstituted aryl, an aryl-C₁₋₃ alkyl, CH₂-indol-3-yl, —(CH₂)₁₋₆SCH₃, —CH₂-imidazol-4-yl, CH(OH)(CH₂)₀₋₅CH₃, —CH₂((4′-OH)-Ph), and wherein R₂₁-R₂₃ can be independently hydrogen or an unsubstituted C₁₋₆ alkyl.

In any one of the forms of Formula I described above, R₃ and R₄ can be independently hydrogen, a substituted or unsubstituted alkyl, a substituted or unsubstituted aryl, a substituted or unsubstituted hetercyclyl, a substituted or unsubstituted heteroaryl, an alkoxy, an amino, or an imine, preferably R₃ and R₄ are independently hydrogen or a substituted or unsubstituted C₁-C₂₀ alkyl.

In any one of the forms of Formula I described above, R₅ and/or R′₅ can be hydrogen or a substituted or unsubstituted C₁-C₂₀ alkyl.

In any one of the forms of Formula I described above, when substituents are present, the substituents can be independently an unsubstituted C₁-C₆ alkyl, a C₁-C₆ alkyl substituted with unsubstituted C₁₋₆ alkyl, an unsubstituted C₁-C₆ heteroalkyl, a C₁-C₆ heteroalkyl substituted with unsubstituted C₁₋₆ alkyl, an unsubstituted C₂-C₆ alkenyl, an unsubstituted C₂-C₆ alkynyl, an unsubstituted aryl, an unsubstituted heteroaryl, an unsubstituted C₁-C₆ alkoxy, —(CH₂)₁₋₆CO₂R₂₁, a halogen, C₁-C₆ haloalkyl, —NR₂₂R₂₃, C₁₋₆ acylamino, —NHSO₂C₁₋₆ alkyl, —SO₂NR₂₂R₂₃, —SO₂C₁₋₆ alkyl, —COOR₂₁, —CONR₂₂R₂₃, nitro, cyano, hydroxide, thiol, or an aryl or heteroaryl substituted with unsubstituted C₁_₅ alkyl, an alkoxy, a di(C₁₋₆ alkyl)-amino, a fluoro, or an unsubstituted C₃-C₆ cycloalkyl.

In some forms of Formula I, when a peptide is present, the peptide can be any one of the peptides listed in Table 1.

In some forms, the prodrug is not any one of the compounds listed in Table 2.

In some forms, the prodrug has the following structure:

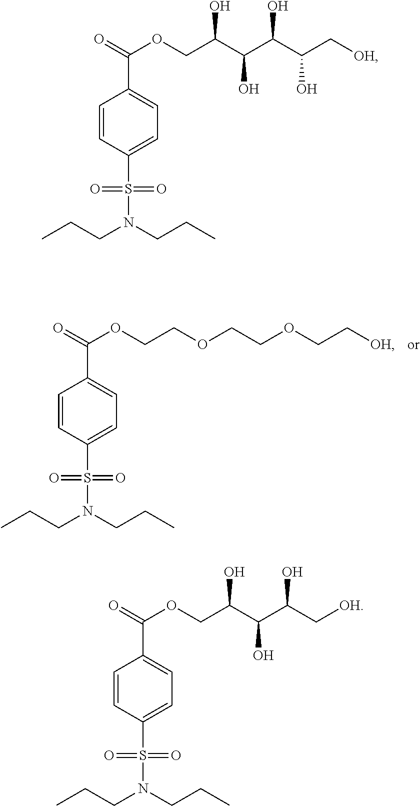 [SMILES: CCCN(CCC)S(=O)(=O)c1ccc(C(=O)OC[C@@H](O)[C@H](O)[C@@H](O)[C@@H](O)CO)cc1 ; CCCN(CCC)S(=O)(=O)c1ccc(C(=O)OCCOCCOCCO)cc1 ; CCCN(CCC)S(=O)(=O)c1ccc(C(=O)OC[C@@H](O)[C@H](O)[C@@H](O)CO)cc1]

Any of the disclosed compounds including probenecid, a metabolite or analog, a prodrug thereof including the prodrugs described herein can be in a delivery vehicle. In some embodiments, the delivery vehicle is nanoparticles or liposomes.

Any of the disclosed compounds alone or in a delivery vehicle can be in a composition such a pharmaceutical composition or a feed composition. A pharmaceutical composition typically includes a pharmaceutically acceptable carrier and/or excipient. For example, pharmaceutical formulations containing one or more compounds selected from probenecid, a metabolite or analog, a prodrug thereof including the prodrugs described herein, and pharmaceutically acceptable salts thereof, and a pharmaceutically acceptable excipient and/or carrier are also disclosed. Feed compositions, which include e.g., commercial agricultural feeds, can include, proteins, fats, sugars, amino acids, minerals, starch, vitamins, and the like. The one or more compounds can be in an effective amount to prevent, treat, or ameliorate one or more symptoms associated with a viral infection in a subject in need thereof. The formulations may further contain one or more additional active agents. The one or more additional active agents can be one or more antiviral and/or anti-inflammatory agents.

In some forms, the pharmaceutically acceptable carrier can be nanoparticles, liposomes, cyclodextrins, or hydrogels, and optionally wherein the one or more prodrugs are encapsulated in, conjugated to, and/or complexed with the nanoparticles, liposomes, cyclodextrins, or hydrogels. In some forms, the pharmaceutical formulation can be in the form of tablets, syrups, capsules, powders, or microneedles.

Methods of treatment are also provided. For example, a method of treating a subject for gout can include administering a subject in need thereof an effective amount of any of the disclosed prodrugs. Methods of treating a subject for hyperuricaemia are also provided and can include administering a subject in need thereof an effective amount of the disclosed prodrugs.

Also provided are methods of treating or preventing a viral infection in a subject. The methods can include administering the subject an effective amount containing one or more compounds selected from probenecid, a metabolite or analog, a prodrug thereof including the prodrugs described herein, and pharmaceutically acceptable salts thereof, or a pharmaceutical composition thereof. Subjects include, but are not limited to humans, non-human mammals, and birds. The subject can be adults or children of any age. In some embodiments, the subjects are human children under 18, for example, from 2-10 years old inclusive. The viral infection can be due to DNA or RNA viruses. Exemplary DNA viruses include those belonging to the families adenoviridae, papoviridae, herpesviridae, poxviridae, anelloviridae, and pleolipoviridae. Exemplary RNA viruses include those belonging to the families Reoviridae, Picornaviridae, Caliciviridae, Togaviridae, Arenaviridae, Flaviviridae, Orthomyxoviridae, Paramyxoviridae, Bunyaviridae, Rhabdoviridae, Filoviridae, Coronaviridae, Astroviridae, Bornaviridae, Arteriviridae, and Hepeviridae. For example, in some embodiments, the virus is from a negative-sense RNA virus family such as Arenaviridae, Bunyaviridae, Filovirida, Nymaviridae, Orthmyxoviridae, Paramyxoviridae, Pneumoviridae, or Rhabdoviridae; or a positive strand family such as Arteriviridae, Astroviridae, Caliciviridae, Coronaviridae, Flaviviridae, Hepeviridae/Nodaviridae, Picornaviridae, orTogaviridae.

In some embodiments, the virus is a respiratory virus.

In some embodiments, the virus is selected from influenza viruses, optionally influenza virus A, influenza virus B, or influenza virus C, respiratory syncytial virus (RSV), human metapneumovirus, coronaviruses, measles virus, parainfluenza virus, mumps virus, Zika virus, dengue virus, yellow fever virus, Japanese encephalitis virus, West Nile virus, Hepatitis A virus, Hepatitis B virus, or Hepatitis C virus.

In some embodiments, the virus is a coronavirus, optionally selected from a Severe acute respiratory syndrome-related coronavirus, a Bat Hp-betacoronavirus Zhejiang2013, a Rousettus bat coronavirus GCCDCl, a Rousettus bat coronavirus HKU9, a Eidolon bat coronavirus C704, a Pipistrellus bat coronavirus HKU5, a Tylonycteris bar coronovirus HKU4, a Middle East respiratory syndrome-related coronavirus, a Hedgehog coronavirus, a murine coronavirus, a Human coronavirus HKU1, a China Rattus coronavirus HKU24, a Betacoronavirus 1, a Myodes coronavirus 2JL14, a Human coronavirus NL63, a Human coronavirus 229E, or a Human coronavirus OC43.

In particular embodiments, the coronavirus is a Severe acute respiratory syndrome-related coronavirus such as SARS-CoV-2, SARS-CoV, SARSr-CoV RaTG13, SARS-CoV PC4-227, or SARSr-CoV BtKY72.

In some embodiments, the subject has COVID-19.

In some embodiments, the virus has an RNA genome optionally encoding an RNA-dependent RNA polymerase (RdRp).

The virus can be a member of the kingdom Orthornavirae. In some embodiments, the virus utilizes a host organic anion transporter optionally selected from OAT1, OAT2, OAT3, OAT4, OAT5, OAT6, OAT7, rOAT8, OAT9, OAT10, and/or URAT1.

In some embodiments, the subject has one or more symptoms are selected from fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhea, tiredness, nausea, anosmia, skin rash, and combinations thereof. In some embodiments, the subject is asymptomatic.

In some embodiments, the compound is administered systemically, locally, or regionally. In some embodiments, the compound is administered orally, parenterally, topically, or mucosally. In particular embodiments, the compound is administered mucosally to the lungs, nasal mucosa, or combination thereof. In some embodiments, the compound is administered prophylactically and/or therapeutically to animals, particularly agricultural animals such as chickens, cattle, and/or pigs, or domesticated animals such as dogs or cats.

Dosage and dosage regimens are also provided. For example, in some embodiments, the compound is administered in an effective amount to reduce viral replication. Exemplary dosages include, but are not limited to, 10 mg-2,000 mg, or 600 mg, 900 mg, or 1,800 mg, optionally twice daily, optionally for 14 days or more. In more particular embodiments, the dose is 250 mg to 2,000 mg once or twice a day, for example, 600 mg or 900 mg twice a day, or 1,800 mg once a day. In some embodiments, the subject is treated by pulse dosing. The subject can be a mammal such as a human.

### BRIEF DESCRIPTION OF THE DRAWINGS

FIG. 1 is a graph of probenecid prophylaxis from RSV A2. Cell lines were prophylactically treated with probenecid 24 h before RSV A2 infection. Probenecid prophylaxis significantly (**** p<0.0001) inhibited the virus replication in Vero E6 cells, HEp-2 cells, and NHBE cells compared to control (DMSO only). Viral titers were determined by plaque assay. The IC50 and IC90 values are shown in Table 3.

FIG. 2 is a graph of probenecid therapy from RSV A2. Cell lines were treated with probenecid 24 h after RSV A2 infection. Treatment significantly (**** p<0.0001) inhibited the virus replication in Vero E6 cells, HEp-2 cells, and NHBE cells compared to control (DMSO only). Viral titers were determined by plaque assay. The IC50 and IC90 values are shown in Table 3.

FIG. 3 is a graph of probenecid prophylaxis from RSV B1. Cell lines were prophylactically treated with probenecid 24 h before RSV B1 infection. Probenecid prophylaxis significantly (**** p<0.0001) inhibited the virus replication in Vero E6 cells, HEp-2 cells, and NHBE cells compared to control (DMSO only). Viral titers were determined by plaque assay. The IC50 values are shown in Table 3, IC90 values are not available as the virus was not reduced by 90%.

FIG. 4 is a graph of probenecid therapy from RSV B1. Cell lines were treated with probenecid 24 h after RSV B1 infection. Treatment significantly (**** p<0.0001) inhibited the virus replication in Vero E6 cells, HEp-2 cells, and NHBE cells compared to control (DMSO only). Viral titers were determined by plaque assay. The IC50 values are shown in Table 1, IC90 values are not available, as the virus was not reduced by 90%.

FIG. 5 is a graph of probenecid prophylaxis from Memphis-37. Cell lines were prophylactically treated with probenecid 24 h before RSV Memphis-37 infection. Probenecid prophylaxis significantly (**** p<0.0001) inhibited the virus replication in Vero E6 cells, HEp-2 cells, and NHBE cells compared to control (DMSO only). Viral titers were determined by plaque assay. The IC50 and IC90 values are shown in Table 3.

FIG. 6 is a graph of probenecid therapy from Memphis-37. Cell lines were treated with probenecid 24 h after RSV Memphis-37 infection. Treatment significantly (**** p<0.0001) inhibited the virus replication in Vero E6 cells, HEp-2 cells, and NHBE cells compared to control (DMSO only). Viral titers were determined by plaque assay. The IC50 and IC90 values are shown in Table 3.

FIG. 7 is a bar graph of lung virus titers from female BALB/c mice. The mice received 2 mg/kg or 200 mg/kg probenecid 24 h before infection (prophylaxis) or 24 h pi (treatment). The mice were i.n. infected with 10⁶ PFU of RSV A2. At days 3, 5, and 7 pi, the lungs were harvested, and virus titers were determined by plaque assay. There is a significant (**** p<0.0001) reduction in lung viral titers with all probenecid treatments compared to control (PBS).

FIG. 8 is a bar graph of lung virus titers from male BALB/c mice. The mice received 2 mg/kg or 200 mg/kg probenecid 24 h before infection (prophylaxis) or 24 h pi (treatment). The mice were i.n. infected with 10⁶ PFU of RSV A2. At days 3, 5, and 7 pi, the lungs were harvested, and virus titers were determined by plaque assay. There is a significant (**** p<0.0001) reduction in lung viral titers with all probenecid treatments compared to control (PBS).

FIG. 9 is a bar graph showing the results of RSV A2 ELISA. Day 7 pi serum from female BALB/c mice (n=4/group) were assayed for antibodies against RSV A2. The graph indicates total IgG, IgG1, or IgG2a determined by specific secondary antibodies. Controls include 131-2A (anti-F protein; IgG2a) or 131-2G (anti-G protein; IgG1), or a mixture of these monoclonal antibodies (total IgG). As expected, there were very low IgG levels as sera derived from primary RSV A2 infection. Bars represent the mean OD₄₅₀ values+SEM of three independent experiments.

FIG. 10 is a graph of probenecid therapy from Mumps (Jones) virus. Cell lines were treated with probenecid 24 h after viral infection. L.O.D.=limit of detection.

FIG. 11 is a graph of probenecid therapy from Zika (MR766) virus. Cell lines were treated with probenecid 24 h after viral infection. L.O.D.=limit of detection.

FIG. 12 is a graph of probenecid therapy from Dengue Type 1 virus. Cell lines were treated with probenecid 24 h after viral infection. L.O.D.=limit of detection.

FIGS. 13A-13F are graphs of probenecid therapy from influenza viruses: influenza A strain Swine/Missouri/2006 (13A, 13B), influenza A strain Vietnam/2004 PR8 (13C, 13D), and influenza B strain B/Malaysia/2506/2004 (13E, 13F). Cell lines were treated with probenecid 24 h after viral infection. L.O.D.=limit of detection.

FIG. 14A is a graph of probenecid prophylaxis from measles virus as a measure of average syncytia per well (log 10) vs. concentration of probenecid (μM). FIGS. 14B and 14C are graphs of probenecid treatment before infection of Hep2 (14B) and Vero cells (14C) with measles virus (MOI=0.01).

FIG. 15A is a flow-chart showing simulation plan for steady-state concentration to reach IC90. FIG. 15B is a flow-chart showing simulation plan for time to reach IC90.

FIG. 16 is graph showing a concentration-time profile of probenecid stratified based on dose administered.

FIGS. 17A-17D are goodness-of-fit plots demonstrating observation versus population (17A) and individual predictions (17B) and conditional weighted residuals (CWRES) versus population prediction (17C) and time (Time after Dose (TAD) (17D).

FIGS. 18A-18C are plots showing observed (DV) and predicted (PRED) plasma concentration-time profiles.

FIG. 19 is a plot of simulated pharmacokinetic (PK) profiles following repeat dosing of probenecid at 600 and 900 mg.

FIG. 20 is a plot of simulated pharmacokinetic following repeat dosing of probenecid at 1800 mg.

FIGS. 21A-21D are plots of simulated pharmacokinetic (PK) profiles following once daily (qd) repeat dosing of probenecid at 100 mg (21A), 500 mg (21B), 600 mg (21C), and 1800 mg (21D). The IC₉₀ level corrected for 95% protein binding is 2.08 μg/ml (shown as a dashed line).

FIGS. 22A-22D are plots of simulated pharmacokinetic (PK) profiles following twice daily (bid) repeat dosing of probenecid at 500 mg (22A), 600 mg (22B), 900 mg (22C), and 1000 mg (22D). The IC₉₀ level corrected for 95% protein binding is 2.08 μg/ml (shown as a dashed line).

FIGS. 23A-23D are plots showing comparison of once (qd) (23C, 23D) or twice daily (bid) (23A, 23B) administration of probenecid 10 mg/kg in female (23A, 23C) and male (23B, 23D) pediatric subjects (ages 2-10 years).

FIGS. 24A-24D are plots showing comparison of once (qd) (24C, 24D) or twice daily (bid) (24A, 24B) administration of probenecid 20 mg/kg in female (24A, 24C) and male (24B, 24D) pediatric subjects (ages 2-10 years).

FIGS. 25A-25F are plots showing comparison of once (qd) daily administration of probenecid 10 mg/kg (25A, 25B), 20 mg/kg (25C, 25D), and 25 mg/kg (25E, 25F) in female (25A, 25C, 25E) and male (25B, 25D, 25F) pediatric subjects (ages 2-13 years).

FIG. 26A-26B are plots showing plaque assays results following prophylactic (24 h prior to) or post-infection (24 h post) (“treatment”) probenecid treatment of BALB/c male (26A) and female (26B) mice challenged intranasally with 1e6 PFU hMPV CAN83 (A2 strain). Mice (n=5/sex/group).

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

“Substituted alkyl” refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, substituted or unsubstituted carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF₃, —CH₂—CF₃, —CCl₃); —CN; —NCOCOCH₂CH₂; —NCOCOCHCH; and —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, substituted or unsubstituted carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom on the carbon backbone. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure.

Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C═C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond.

Asymmetric structures such as (AB)C≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

“Aryl,” as used herein, refers to C₅-C₂₆-membered aromatic or fused aromatic ring systems. Examples of aromatic groups are benzene, naphthalene, anthracene, phenanthrene, chrysene, pyrene, corannulene, coronene, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, substituted or unsubstituted carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle” and “heterocyclyl” are used interchangeably, and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₂₆-membered aromatic or fused aromatic ring systems, in which one or more carbon atoms on one or more aromatic ring structures have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, substituted or unsubstituted carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a chemical moiety that includes two or more fused aryl groups. When two or more fused heteroaryl groups are involved, the chemical moiety can be referred to as a “polyheteroaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “cyclic ring” or “cyclic group” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, or a substituted or unsubstituted heterocyclyl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, and heterocyclyls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The term “alkoxyl” or “alkoxy” generally describe compounds represented by the formula —OR^{v}, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of —O-alkyl, —O-alkenyl, —O-alkynyl, —O-arakyl, —O-aryl, —O-heteroaryl, —O-polyaryl, —O-polyheteroaryl, —O-heterocyclyl, etc. When R^{v} is a substituted group, one or more substituents replace one or more hydrogen atoms on one or more carbons of R^{v}. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA¹, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

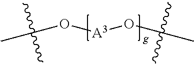

where A³ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized and refers to a compound of the formula —OR^{v} wherein R^{v} is C₆H₅ (i.e., —O—C₆H₅). One of skill in the art recognizes that a phenoxy is a species of the alkoxyl genus and aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein. One of skill in the art recognizes that an aroxy or aryloxy is a species of the alkoxyl genus.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

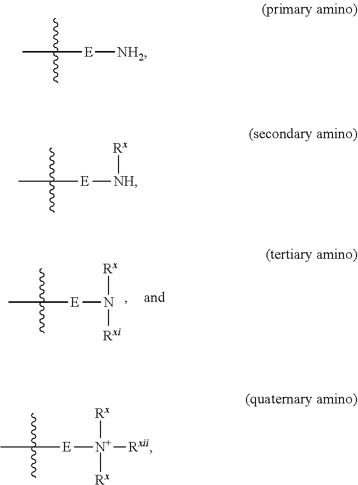

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The terms “amide” or “amido” are used interchangeably, refer to both “unsubstituted amido” and “substituted amido” and are represented by the general formula:

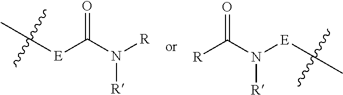

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed. It is understood by those of ordinary skill in the art, that the E groups listed above are divalent (e.g., methylene, ethane-1,2-diyl, ethene-1,2-diyl, 1,4-phenylene, cyclohexane-1,2-diyl).

“Carbonyl,” as used herein, is art-recognized and includes such moieties as can be represented by the general formula:

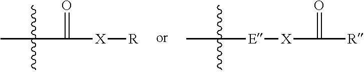

wherein X is a bond, or represents an oxygen or a sulfur, and R represents a hydrogen, unsubstituted alkyl, unsubstituted alkenyl, unsubstituted alkynyl, unsubstituted heterocyclyl, unsubstituted aralkyl (e.g. unsubstituted alkylaryl, unsubstituted arylalkyl), unsubstituted aryl, unsubstituted heteroaryl, unsubstituted polyaryl, unsubstituted polyheteroaryl, unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is unsubstituted alkylene, unsubstituted alkyl, unsubstituted alkenyl, unsubstituted alkynyl, unsubstituted aralkyl, unsubstituted aryl, unsubstituted heteroaryl, unsubstituted polyaryl, unsubstituted polyheteroaryl, unsubstituted heterocyclyl; R′ represents a hydrogen, unsubstituted alkyl, unsubstituted alkenyl, unsubstituted alkynyl, unsubstituted heterocyclyl, unsubstituted aralkyl (e.g. unsubstituted alkylaryl, unsubstituted arylalkyl), unsubstituted aryl, unsubstituted heteroaryl, unsubstituted polyaryl, unsubstituted polyheteroaryl, unsubstituted heterocyclyl, a hydroxyl, an alkoxy, a phosphonium, a phosphanyl, an amido, an amino, or —(CH₂)ₘ—R″; R″ represents a hydroxyl group, unsubstituted aryl, unsubstituted cycloalkyl, unsubstituted cycloalkenyl, unsubstituted heterocyclyl, unsubstituted aryl, unsubstituted heteroaryl, unsubstituted polyaryl, unsubstituted polyheteroaryl, an alkoxy, a phosphonium, a phosphanyl, an amido, or an amino; and m is zero or an integer ranging from 1 to 8. It is understood by those of ordinary skill in the art, that the E″ groups listed above are divalent (e.g., methylene, ethane-1,2-diyl, ethene-1,2-diyl, 1,4-phenylene, cyclohexane-1,2-diyl). Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The term “substituted carbonyl” refers to a carbonyl, as defined above, wherein one or more hydrogen atoms in R, R′, and/or E″ are independently substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. It is understood by those of ordinary skill in the art, that the E and E″ groups listed above are divalent (e.g., methylene, ethane-1,2-diyl, ethene-1,2-diyl, 1,4-phenylene, cyclohexane-1,2-diyl).

The term “phosphanyl” is represented by the formula

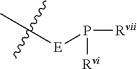

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonium” is represented by the formula

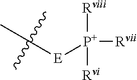

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi}, R^{vii}, and R^{viii} taken together with the P⁺ atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonyl” is represented by the formula

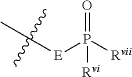

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfinyl” is represented by the formula

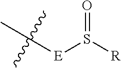

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonyl” is represented by the formula

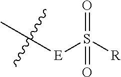

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R′″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfamoyl” refers to a sulfonamide or sulfonamide represented by the formula

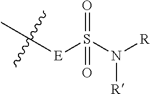

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “silyl group” as used herein is represented by the formula —SiRR′R,″ where R, R′, and R″ can be, independently, a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above. Such substituents can be any substituents described above, e.g., halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “phenylthio” is art recognized, and refers to —S—C₆H₅, i.e., a phenyl group attached to a sulfur atom. One of skill in the art recognizes that a phenylthio is a species of the thiol genus.

The term “substituted phenylthio” refers to a phenylthio group, as defined above, having one or more substituents replacing a hydrogen on one or more carbons of the phenyl ring. Such substituents include, but are not limited to, halogen, azide, alkyl, aralkyl, alkenyl, alkynyl, cycloalkyl, hydroxyl, substituted or unsubstituted carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

“Analog” as relates to a given compound, refers to another compound that is structurally similar, functionally similar, or both, to the specified compound. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered an analog of a specified compound, if it has a Tanimoto coefficient with the specified compound between 0.5 and 1.0, inclusive, preferably between 0.7 and 1.0, inclusive, most preferably between 0.85 and 1.0, inclusive. A compound is functionally similar to a specified, if it induces the same pharmacological effect, physiological effect, or both, as the specified compound. “Analog” can also refer to a modification including, but not limited to, hydrolysis, reduction, or oxidation products, of the disclosed compounds. Hydrolysis, reduction, and oxidation reactions are known in the art.

As used herein, the terms “individual”, “host”, “subject”, and “patient” are used interchangeably herein, and refer animals, particularly birds and mammals, including, but not limited to, primates such as humans, bats, rodents, such as mice and rats, and other laboratory animals, or agricultural or domesticated animals.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Numerical ranges disclose individually each possible number that such a range could reasonably encompass, as well as any sub-ranges and combinations of sub-ranges encompassed therein. For example, in a given range carbon range of C₁-C₆, the range also discloses C₁, C₂, C₃, C₄, C₅, and C₆, as well as any subrange between these numbers (for example, C₃-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Compositions

Probenecid, metabolites, analogs, prodrugs, and pharmaceutically acceptable salts thereof including, but not limited to a sodium salt, of any of the foregoing; compositions and formulations thereof; and methods of use thereof including, but not limited to, the prevention and treatment of viral infections are provided. The disclosed methods typically include administering a subject in need thereof an effective amount of probenecid, a metabolite, analog, or prodrug thereof, or a pharmaceutically acceptable salt thereof, including, but not limited to a sodium salt thereof.

### A. Probenecid & Metabolites, Analogs, and Prodrugs Thereof

Probenecid (4-[(dipropylamino) sulfonyl] benzoic acid (CAS No. 57-66-9)) has the structure:

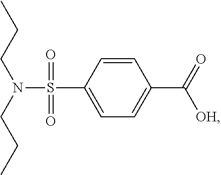 [SMILES: CCCN(CCC)S(=O)(=O)c1ccc(C(=O)O)cc1]

and has been sold under the brand names BENEMID® and PROBALAN®.

Probenecid is a highly lipid soluble benzoic acid derivative with an excellent safety profile that was developed in the 1950's to decrease the renal tubular excretion of penicillin. Probenecid, USP is a white or nearly white, fine, crystalline powder. Probenecid is soluble in dilute alkali, in alcohol, in chloroform, and in acetone; it is practically insoluble in water and in dilute acids. It has a half life of 6-12 hours. See also Drugbank Accession Number DB01032 (APRD00167), and PubChem CID 4911.

Metabolites and analogs of probenecid are known, see, for example, Guarino, et al., “Mass spectral identification of probenecid metabolites in rat bile,” Eur. J. Pharmacol., 8, 244-252 (1969), Perel, et al., “Identification and renal excretion of probenecid metabolites in man,” Life Sciences, 9, 23, 1337-1343 (1970), Perel, et al., “Studies of the renal excretion of probenecid acyl glucuronide in man,” Eur. J. Clin. Pharmacol, 3, 106-112 (1971), Dayton and Perel, “The metabolism of probenecid in man,”. N. Y. Acad. Sci., 179, 399-402 (1971), Dayton, et al., “The effect of probenecid, phenylbutazone and their analogues on the excretion of L-ascorbic acid in rats,” J. Med. Chem. 9, 941-944 (1966), and Israili, et al., “Metabolites of probenecid. Chemical, physical, and pharmacological studies,” J. Med. Chem., 15, 7, 709-713 (1972), each of which is specifically incorporated by reference in its entirety.

In some embodiments, the metabolite is a glucuronide derivative of probenecid such as acyl glucuronide or a R-ether glucuronide.

Exemplary probenecid metabolites and analogs include, but are not limited to,
dl-p-(N-propyl-N-2-hydroxypropylsulfamoyl)benzoic acid,
p-(N-Propyl-N-3-hydroxypropylsulfamoyl)benzoic acid,
p-(N-propyl-N-3-propionitrilosulfamoyl)benzoic acid,
p-(N-propyl-N-2-carboxyethylsulfamoyl)benzoic acid,
p-(N-propylsulfamoyl)benzoic acid,
p-(N,N-pentamethylenesulfamoyl)benzoic acid (piperidyl analog),
p-(N-propyl-N-2-propenylsulfamoyl)benzoic acid,
p-(N-propyl-N-2-oxopropylsulfamoyl)benzoic acid,
p-sulfamoylbenzoic acid,
p-(N-methylsulfamoyl)benzoic acid,
p-(N-ethylsulfamoyl)benzoic acid,
p-(N-hexylsulfamoyl)benzoic acid,
p-(N-cyclohexylsulfamoyl)benzoic acid,
p-(N-dimethylsulfamoyl)benzoic acid,
p-(N-methyl,N-ethylsulfamoyl)benzoic acid,
p-(N,N-diethylsulfamoyl)benzoic acid,
p-(N-ethyl,N-propylsulfamoyl)benzoic acid,
p-(N,N-diisopropylsulfamoyl)benzoic acid,
p-(N,N-dibutylsulfamoyl)benzoic acid,
o-hydroxy-p-(N-dipropylsulfamoyl)benzoic acid,
o-methoxy-p-(N-dipropylsulfamoyl)benzoic acid,
o-nitro-p-(N,N-dipropylsulfamoyl)benzoic acid,
m-nitro-p-(N,N-dipropylsulfamoyl)benzoic acid, and
m-methyl-p-(N,N-dipropylsulfamoyl)benzoic acid.

Other exemplary probenecid-related compounds are listed in Table 1. Thus, in some embodiments, the compound is selected from those listed in Table 1.

TABLE 1
Probenecid and Probenecid Analogs

| Structure Name | Reference |
|---|---|
| Probenecid Methyl | webbook.nist.gov/cgi/ |
| | cbook.cgi?ID=R40461&Units=SI |
| Methyl 4- | pubchem.ncbi.nlm.nih.gov/compound/ |
| [(dipropylamino)sulfonyl]benzoate | 800079 |
| Probenecid Sodium | pubchem.ncbi.nlm.nih.gov/compound/ |
| | 23662399 |
| but-3-enyl 4- | pubchem.ncbi.nlm.nih.gov/compound/ |
| (dipropylsulfamoyl)benzoate | 146680789 |
| but-3-enyl 4- | nature.com/articles/s41557-020-0541-1 |
| (dipropylsulfamoyl)benzoate | |
| Iodomethyl 4- | pubchem.ncbi.nlm.nih.gov/compound/ |
| (dipropylsulfamoyl)benzoate | 23449285#section=Structures |
| Chloromethyl 4-(N,N- | pubchem.ncbi.nlm.nih.gov/compound/ |
| dipropylsulfamoyl)benzoat | 21567634 |
| Ethyl 4-(dipropylsulfamoyl)benzoate | pubchem.ncbi.nlm.nih.gov/compound/ |
| | 17811092#section=2D-Structure |
| 2-(6-Morpholin-4-yl-1,3- | pubchem.ncbi.nlm.nih.gov/compound/ |
| dioxobenzo[de]isoquinolin-2-yl)ethyl 4- | 4277062 |
| (dipropylsulfamoyl)benzoate | |
| [(2R,3S,5R)-5-[5-[(E)-2-Bromoethenyl]- | pubchem.ncbi.nlm.nih.gov/compound/ |
| 3-[4-(dipropylsulfamoyl)benzoyl]-2,4- | 88740230 |
| dioxopyrimidin-1-yl]-3-[4- | |
| (dipropylsulfamoyl)benzoyl]oxyoxolan- | |
| 2-yl]methyl 4- | |
| (dipropylsulfamoyl)benzoate | |
| (2,2-Dichloro-3-{[4- | pubchem.ncbi.nlm.nih.gov/compound/ |
| (dipropylsulfamoyl)benzoyloxy]methyl} | 4121940 |
| cyclopropyl)methyl 4- | |
| (dipropylsulfamoyl)benzoate | |
| [3-[(2R,3S,5R)-5-[5-[(E)-2- | pubchem.ncbi.nlm.nih.gov/compound/ |
| Bromoethenyl]-2,4-dioxopyrimidin-1- | 88740084#section=2D-Structure |
| yl]-3-[2-[4- | |
| (dipropylsulfamoyl)benzoyl]oxyacetyl]- | |
| 3-hydroxyoxolan-2-yl]-3-hydroxy-2- | |
| oxopropyl] 4- | |
| (dipropylsulfamoyl)benzoate | |
| [(2R,3S,5R)-5-[5-[(E)-2-Bromoethenyl]- | pubchem.ncbi.nlm.nih.gov/compound/ |
| 3-[4-(dipropylsulfamoyl)benzoyl]-2,4- | 88740230 |
| dioxopyrimidin-1-yl]-3-[4- | |
| (dipropylsulfamoyl)benzoyl]oxyoxolan- | |
| 2-yl]methyl 4- | |
| (dipropylsulfamoyl)benzoate | |
| [3-[(2S,3S,5R)-5-[5-[(E)-2- | pubchem.ncbi.nlm.nih.gov/compound/ |
| Bromoethenyl]-3-[4- | 88740588 |
| (dipropylsulfamoyl)benzoyl]-2,4- | |
| dioxopyrimidin-1-yl]-3-hydroxyoxolan- | |
| 2-yl]-3-hydroxy-2-oxopropyl] 4- | |
| (dipropylsulfamoyl)benzoate | |
| [(1R,3S)-2,2-Dichloro-3-{[4- | pubchem.ncbi.nlm.nih.gov/compound/ |
| (dipropylsulfamoyl)benzoyloxy]methyl} | 51109367 |
| cyclopropyl]methyl 4- | |
| (dipropylsulfamoyl)benzoate | |
| 3-(Dimethylamino)propyl 4- | pubchem.ncbi.nlm.nih.gov/compound/ |
| (dipropylsulfamoyl)benzoate | 409707 |
| Trimethylsilyl 4- | https://pubchem.ncbi.nlm.nih.gov/compound/ |
| (dipropylsulfamoyl)benzoate | 91742743 |
| [3-[(2R,3R,5R)-5-[5-[(E)-2- | pubchem.ncbi.nlm.nih.gov/compound/ |
| Bromoethenyl]-2,4-dioxopyrimidin-1- | 88740025 |
| yl]-3-hydroxy-3-trimethylsilyloxolan-2- | |
| yl]-3-hydroxy-2-oxopropyl] 4- | |
| (dipropylsulfamoyl)benzoate | |
| [2-[(2R,3S,5R)-5-[5-[(E)-2- | pubchem.ncbi.nlm.nih.gov/compound/ |
| Bromoethenyl]-2,4-dioxopyrimidin-1- | 88740851 |
| yl]-3-hydroxy-2- | |
| (hydroxymethyl)oxolan-3-yl]-2- | |
| oxoethyl] 4- | |
| (dipropylsulfamoyl)benzoate | |
| 2-(4-Bromophenyl)-2-oxoethyl 4- | pubchem.ncbi.nlm.nih.gov/compound/ |
| (dipropylsulfamoyl)benzoate | 17370326 |
| [3-[(2S,3S,5R)-5-[5-[(E)-2- | pubchem.ncbi.nlm.nih.gov/compound/ |
| Bromoethenyl]-2,4-dioxopyrimidin-1- | 88740038 |
| yl]-3-hydroxyoxolan-2-yl]-3-hydroxy-2- | |
| oxopropyl] 4- | |
| (dipropylsulfamoyl)benzoate | |
| [4-[(2S,3S,5R)-5-[5-[(E)-2- | pubchem.ncbi.nlm.nih.gov/compound/ |
| Bromoethenyl]-2,4-dioxopyrimidin-1- | 88740157#section=2D-Structure |
| yl]-3-hydroxyoxolan-2-yl]-4-hydroxy-3- | |
| oxobutan-2-yl] 4- | |
| (dipropylsulfamoyl)benzoate | |
| [(1R,3R)-2,2-dichloro-3-[[4- | pubchem.ncbi.nlm.nih.gov/compound/ |
| (dipropylsulfamoyl)benzoyl]oxymethyl] | 97962996#section=Structures |
| cyclopropyl]methyl 4- | |
| (dipropylsulfamoyl)benzoate | |
| [(1S,3S)-2,2-dichloro-3-[[4- | pubchem.ncbi.nlm.nih.gov/compound/ |
| (dipropylsulfamoyl)benzoyl]oxymethyl]cyclopropyl | 97962997#section=3D-Status |
| methyl 4- | |
| (dipropylsulfamoyl)benzoate | |
| [2-[[(2S,5S)-5-[5-[(E)-2-bromoethenyl]- | pubchem.ncbi.nlm.nih.gov/compound/ |
| 2,4-dioxopyrimidin-1-yl]-3-[2-[4- | 88740085#section=2D-Structure |
| (dipropylsulfamoyl)benzoyl]oxyacetyl]oxyoxolan- | |
| 2-yl]methoxy]-2-oxoethyl] 4- | |
| (dipropylsulfamoyl)benzoate | |
| [2-[[(2S,5S)-5-[5-[(E)-2-Bromoethenyl]- | pubchem.ncbi.nlm.nih.gov/compound/ |
| 3-[4-(dipropylsulfamoyl)benzoyl]-2,4- | 88740589#section=Structures |
| dioxopyrimidin-1-yl]-3-hydroxyoxolan- | |
| 2-yl]methoxy]-2-oxoethyl] 4- | |
| (dipropylsulfamoyl)benzoate | |
| 2-Oxo-2-phenylethyl 4- | pubchem.ncbi.nlm.nih.gov/compound/ |
| [(dipropylamino)sulfonyl]benzoate | 1260335 |
| 2-(4-Nitrophenyl)-2-oxoethyl 4- | pubchem.ncbi.nlm.nih.gov/compound/ |
| [(dipropylamino)sulfonyl]benzoate | 1273942 |
| [1-(2-Hydroxyphenyl)ethylideneamino] | pubchem.ncbi.nlm.nih.gov/compound/ |
| 4-(dipropylsulfamoyl)benzoate | 2803566 |
| [[Amino(1,3-benzodioxol-5- | pubchem.ncbi.nlm.nih.gov/compound/ |
| yl)methylidene]amino] 4- | 2803598 |
| (dipropylsulfamoyl)benzoate | |
| Magnesium; 4- | pubchem.ncbi.nlm.nih.gov/compound/ |
| (dipropylsulfamoyl)benzoate | 3015457#section=2D-Structure |
| [(Z)-[Amino(1,3-benzodioxol-5- | pubchem.ncbi.nlm.nih.gov/compound/ |
| yl)methylidene]amino] 4- | 5713542 |
| (dipropylsulfamoyl)benzoate | |
| [3-[(3- | pubchem.ncbi.nlm.nih.gov/compound/ |
| Phenylphenyl)methylcarbamoyl]naphthalen- | 11215720 |
| 2-yl] 4- | |
| (dipropylsulfamoyl)benzoate | |
| [3-(naphthalen-1- | pubchem.ncbi.nlm.nih.gov/compound/ |
| ylmethylcarbamoyl)naphthalen-2-yl] 4- | 11238722#section=Names-and-Identifiers |
| (dipropylsulfamoyl)benzoate | |
| [3-(1,3-Thiazol-2- | pubchem.ncbi.nlm.nih.gov/compound/ |
| ylcarbamoyl)naphthalen-2-yl] 4- | 11432642 |
| (dipropylsulfamoyl)benzoate | |
| potassium; 4- | pubchem.ncbi.nlm.nih.gov/compound/ |
| (dipropylsulfamoyl)benzoate | 71727220#section=3D-Conformer |
| [1-(1,3-benzothiazol-2-yl)azetidin-3-yl] | pubchem.ncbi.nlm.nih.gov/compound/ |
| 4-(dipropylsulfamoyl)benzoate | 90606878 |
| 1-nitrooxyethyl 4- | pubchem.ncbi.nlm.nih.gov/compound/ |
| (dipropylsulfamoyl)benzoate | 118520564#section=IUPAC-Name |
| [(2S,5R)-5-(6-oxo-1H-purin-9- | pubchem.ncbi.nlm.nih.gov/compound/ |
| yl)oxolan-2-yl]methyl 4- | 145998147#section=Computed-Descriptors |
| (dipropylsulfamoyl)benzoate | |
| [(2S,5R)-5-(6-oxo-1H-purin-9- | library.ua.edu/vwebv/holdingsInfo?bibId=761592 |
| yl)oxolan-2-yl]methyl 4- | |
| (dipropylsulfamoyl)benzoate | |
| (2,5-dioxopyrrolidin-1-yl) 4- | pubchem.ncbi.nlm.nih.gov/compound/ |
| (dipropylsulfamoyl)benzoate | 124286370#section=Molecular-Formula |
| (furan-2-carbonylamino) 4- | pubchem.ncbi.nlm.nih.gov/compound/ |
| (dipropylsulfamoyl)benzoate | 134118298#section=Names-and-Identifiers |
| [(E)-1-(2- | pubchem.ncbi.nlm.nih.gov/compound/ |
| hydroxyphenyl)ethylideneamino] 4- | 135520584#section=Names-and-Identifiers |
| (dipropylsulfamoyl)benzoate | |
| (3-methyl-3-propoxybutyl) 4- | pubchem.ncbi.nlm.nih.gov/compound/ |
| (dipropylsulfamoyl)benzoate | 143379281#section=Names-and-Identifiers |
| 3-(Dimethylamino)propyl 4- | pubchem.ncbi.nlm.nih.gov/compound/ |
| (dipropylsulfamoyl)benzoate | 409707#section=Structures |
| (2-methoxy-6-methylphenyl) 4- | pubchem.ncbi.nlm.nih.gov/compound/ |
| (dipropylsulfamoyl)benzoate | 2797739#section=IUPAC-Name |
| (cyclohexylideneamino) 4- | pubchem.ncbi.nlm.nih.gov/compound/ |
| (dipropylsulfamoyl)benzoate | 2803455#section=Names-and-Identifiers |
| [(E)-[amino(1,3-benzodioxol-5- | pubchem.ncbi.nlm.nih.gov/compound/ |
| yl)methylidene]amino] 4- | 5911623 |
| (dipropylsulfamoyl)benzoate | |
| [3-[(2- | pubchem.ncbi.nlm.nih.gov/compound/ |
| phenylphenyl)methylcarbamoyl]naphthalen- | 11169529#section=Names-and-Identifiers |
| 2-yl] 4- | |
| (dipropylsulfamoyl)benzoate | |
| [3-(benzylcarbamoyl)naphthalen-2-yl] | pubchem.ncbi.nlm.nih.gov/compound/ |
| 4-(dipropylsulfamoyl)benzoate | 11478320#section=Names-and-Identifiers |
| (2-Fluoro-3-phenylprop-2-enoyl) 4- | pubchem.ncbi.nlm.nih.gov/compound/ |
| (dipropylsulfamoyl)benzoate | 57258224 |
| 2-(2-hydroxyethoxy)ethyl 4- | pubchem.ncbi.nlm.nih.gov/compound/ |
| (dipropylsulfamoyl)benzoate | 58833024#section=3D-Conformer |
| [3-(3-hydroxy-3-methylbutoxy)-3- | pubchem.ncbi.nlm.nih.gov/compound/ |
| methylbutyl] 4- | 71108565#section=Related- |
| (dipropylsulfamoyl)benzoate | Compounds-with-Annotation |
| [(Z)-2-fluoro-3-phenylprop-2-enoyl] 4- | pubchem.ncbi.nlm.nih.gov/compound/ |
| (dipropylsulfamoyl)benzoate | 88206223#section=Names-and-Identifiers |
| [2-[(2S,5S)-5-[5-[(E)-2-bromoethenyl]- | pubchem.ncbi.nlm.nih.gov/compound/ |
| 2,4-dioxopyrimidin-1-yl]-2- | 88740852#section=Names-and-Identifiers |
| (hydroxymethyl)oxolan-3-yl]oxy-2- | |
| oxoethyl] 4- | |
| (dipropylsulfamoyl)benzoate | |
| [1-(6-fluoro-1,3-benzothiazol-2- | pubchem.ncbi.nlm.nih.gov/compound/ |
| yl)azetidin-3-yl] 4- | 90606661#section=Names-and-Identifiers |
| (dipropylsulfamoyl)benzoate | |
| 2-hydroxyethyl 4- | pubchem.ncbi.nlm.nih.gov/compound/ |
| (dipropylsulfamoyl)benzoate | 148882150#section=3D-Conformer |
| 3-[3-(5-tert-butyl-2-ethenylphenyl)-4- | pubchem.ncbi.nlm.nih.gov/compound/ |
| cyanophenoxy]propyl 4- | 153525185#section=3D-Status |
| (dipropylsulfamoyl)benzoate | |
| [2-[(2S,5S)-5-[5-[(E)-2-bromoethenyl]- | pubchem.ncbi.nlm.nih.gov/compound/ |
| 2,4-dioxopyrimidin-1-yl]-2- | 88740852#section=Patents |
| (hydroxymethyl)oxolan-3-yl]oxy-2- | |
| oxoethyl] 4- | |
| (dipropylsulfamoyl)benzoate | |
| 5-methylsulfanylpentyl 4- | pubchem.ncbi.nlm.nih.gov/compound/ |
| (dipropylsulfamoyl)benzoate | 134128377#section=Names-and-Identifiers |
| [(Z)-1-(2- | pubchem.ncbi.nlm.nih.gov/compound/ |
| hydroxyphenyl)ethylideneamino] 4- | 135781369 |
| (dipropylsulfamoyl)benzoate | |
| 2-[7-bromo-2- | pubchem.ncbi.nlm.nih.gov/compound/ |
| [methyl(methylsulfonyl)amino]-1- | 141735897#section=Computed-Descriptors |
| benzoxepin-3-yl]ethyl 4- | |
| (dipropylsulfamoyl)benzoate | |
| nitrooxymethyl 4- | pubchem.ncbi.nlm.nih.gov/compound/ |
| (dipropylsulfamoyl)benzoate | 144670166#section=Names-and-Identifiers |
| but-3-enyl 4- | pubchem.ncbi.nlm.nih.gov/compound/ |
| (dipropylsulfamoyl)benzoate | 146680789#section=Literature |
| but-3-enyl 4- | nature.com/articles/s41557-020-0541-1 |
| (dipropylsulfamoyl)benzoate | |
| 2-[(15R,19R)-16,18-dioxo-17- | pubchem.ncbi.nlm.nih.gov/compound/ |
| azapentacyclo[6.6.5.02,7.09, 14.015,19] | 1548553#section=3D-Conformer |
| nonadeca-2,4,6,9,11,13-hexaen-17- | |
| yl]ethyl 4-(dipropylsulfamoyl)benzoate | |
| Probenecid Isopropyl Ester | pubchem.ncbi.nlm.nih.gov/compound/ |
| | Probenecid-Isopropyl- |
| | Ester#section=Structures |
| Probenecid Acyl β-D-Glucuronide Benzyl | scbt.com/p/probenecid-acyl-beta-d- |
| Ester | glucuronide-benzyl-ester |
| Probenecid Acyl β-D-Glucuronide | molcan.com/products/10684 |
| Probenecidoxymethyl | US-3931150-A |
| AND probenecidoxy-1-ethyl ester | |
| OR | |
| chloromethyl 4- | |
| (dipropylsulfamoyl)benzoate | |
| 3-[(3-{[4- | WO-2004099127-A1 |
| (dipropylsulfamoyl)phenyl]carbonyloxy}naphthalen- | |
| 2-yl)carbonyloxy]naphthalene-2- | |
| carboxylic acid | |
| ethyl 4-(dipropylsulfamoyl)benzoate | US-20030149016-A1 |
| ethyl 4-(dipropylsulfamoyl)benzoate | US-6677331-B2 |
| AND | |
| 1-iodoethyl 4- | |
| (dipropylsulfamoyl)benzoate | |
| ethyl 4-(dipropylsulfamoyl)benzoate | EP-1101766-A1 |
| 1-iodoethyl 4- | US-20030149016-A1 |
| (dipropylsulfamoyl)benzoate | |
| 2-(2-hydroxyethoxy)ethyl 4- | US-20070254859-A1 |
| (dipropylsulfamoyl)benzoate | |
| 2-(2-hydroxyethoxy)ethyl 4- | US-20070060610-A1 |
| (dipropylsulfamoyl)benzoate | |
| Methyl 4- | WIPO Appl. No. 1020190115018 |
| [(dipropylamino)sulfonyl]benzoate | |
| Methyl 4- | WIPO Appl. No 201910683788.3 |
| [(dipropylamino)sulfonyl]benzoate | |
| [3-[(2R,3S,5R)-5-[5-[(E)-2- | EP0097039A1 |
| Bromoethenyl]-2,4-dioxopyrimidin-1- | |
| yl]-3-[2-[4- | |
| (dipropylsulfamoyl)benzoyl]oxyacetyl]- | |
| 3-hydroxyoxolan-2-yl]-3-hydroxy-2- | |
| oxopropyl] 4- | |
| (dipropylsulfamoyl)benzoate | |
| AND | |
| [3-[(2S,3S,5R)-5-[5-[(E)-2- | |
| Bromoethenyl]-3-[4- | |
| (dipropylsulfamoyl)benzoyl]-2,4- | |
| dioxopyrimidin-1-yl]-3-hydroxyoxolan- | |
| 2-yl]-3-hydroxy-2-oxopropyl] 4- | |
| (dipropylsulfamoyl)benzoate | |
| AND | |
| [3-[(2R,3R,5R)-5-[5-[(E)-2- | |
| Bromoethenyl]-2,4-dioxopyrimidin-1- | |
| yl]-3-hydroxy-3-trimethylsilyloxolan-2- | |
| yl]-3-hydroxy-2-oxopropyl] 4- | |
| (dipropylsulfamoyl)benzoate | |
| AND | |
| [2-[(2R,3S,5R)-5-[5-[(E)-2- | |
| Bromoethenyl]-2,4-dioxopyrimidin-1- | |
| yl]-3-hydroxy-2- | |
| (hydroxymethyl)oxolan-3-yl]-2- | |
| oxoethyl] 4- | |
| (dipropylsulfamoyl)benzoate | |
| AND | |
| [3-[(2S,3S,5R)-5-[5-[(E)-2- | |
| Bromoethenyl]-2,4-dioxopyrimidin-1- | |
| yl]-3-hydroxyoxolan-2-yl]-3-hydroxy- | |
| 2-oxopropyl] 4- | |
| (dipropylsulfamoyl)benzoate | |
| AND | |
| [2-[[(2S,5S)-5-[5-[(E)-2- | |
| bromoethenyl]-2,4-dioxopyrimidin-1- | |
| yl]-3-[2-[4- | |
| (dipropylsulfamoyl)benzoyl]oxyacetyl] | |
| oxyoxolan-2-yl]methoxy]-2-oxoethyl] | |
| 4-(dipropylsulfamoyl)benzoate | |
| AND | |
| [2-[[(2S,5S)-5-[5-[(E)-2- | |
| Bromoethenyl]-3-[4- | |
| (dipropylsulfamoyl)benzoyl]-2,4- | |
| dioxopyrimidin-1-yl]-3-hydroxyoxolan- | |
| 2-yl]methoxy]-2-oxoethyl] 4- | |
| (dipropylsulfamoyl)benzoate | |
| AND | |
| [2-[(2S,5S)-5-[5-[(E)-2-bromoethenyl]- | |
| 2,4-dioxopyrimidin-1-yl]-2- | |
| (hydroxymethyl)oxolan-3-yl]oxy-2- | |
| oxoethyl] 4- | |
| (dipropylsulfamoyl)benzoate | |
| AND | |
| [2-[(2S,5S)-5-[5-[(E)-2-bromoethenyl]- | |
| 2,4-dioxopyrimidin-1-yl]-2- | |
| (hydroxymethyl)oxolan-3-yl]oxy-2- | |
| oxoethyl] 4- | |
| (dipropylsulfamoyl)benzoate | |
| [(2R,3S,5R)-5-[5-[(E)-2-Bromoethenyl]- | EP-0082668-A1 |
| 3-[4-(dipropylsulfamoyl)benzoyl]-2,4- | |
| dioxopyrimidin-1-yl]-3-[4- | |
| (dipropylsulfamoyl)benzoyl]oxyoxolan- | |
| 2-yl]methyl 4- | |
| (dipropylsulfamoyl)benzoate | |
| 1-nitrooxyethyl 4- | US20150328323A1 |
| (dipropylsulfamoyl)benzoate | |
| 1-nitrooxyethyl 4- | US20180125986A1 |
| (dipropylsulfamoyl)benzoate | |
| 1-nitrooxyethyl 4- | US9844599B2 |
| (dipropylsulfamoyl)benzoate | |
| [3-(3-hydroxy-3-methylbutoxy)-3- | WO2007115181A2 |
| methylbutyl] 4- | |
| (dipropylsulfamoyl)benzoate | |
| [(Z)-2-fluoro-3-phenylprop-2-enoyl] 4- | EP1091958A1 |
| (dipropylsulfamoyl)benzoate | |
| [(Z)-2-fluoro-3-phenylprop-2-enoyl] 4- | WO2000001692A1 |
| (dipropylsulfamoyl)benzoate | |
| 2-hydroxyethyl 4- | CN-106470981-B |
| (dipropylsulfamoyl)benzoate | |
| 3-[3-(5-tert-butyl-2-ethenylphenyl)-4- | CN110845363 |
| cyanophenoxy]propyl 4- | |
| (dipropylsulfamoyl)benzoate | |
| 2-[7-bromo-2- | CN109096241 |
| [methyl(methylsulfonyl)amino]-1- | |
| benzoxepin-3-yl]ethyl 4- | |
| (dipropylsulfamoyl)benzoate | |
| nitrooxymethyl 4- | WO2014111957 |
| (dipropylsulfamoyl)benzoate | |

Probenecid is soluble in dilute alkali, in alcohol, in chloroform, and in acetone; it is practically insoluble in water and in dilute acids. Probenecid is completely absorbed after oral administration, where peak plasma concentration is reached in 2-4 hr. The half-life of the drug in plasma is dose dependent and varies from less than 5 hr to more than 8 hr. It is often administered in the form of high dose tablets and has less patient compliance especially in pediatric patients.

Synthetic compounds that can be used as prodrugs of probenecid or probenecid analogs have been developed. These compounds (also referred to herein as “prodrugs”) are inactive forms prepared from active probenecid or probenecid analogs, which can release probenecid or probenecid analogs in their active forms after being administered into a subject in need thereof. For example, the prodrug is an inactive form of probenecid and can be converted to probenecid by chemical or enzymatic cleavages in the body of the subject. The released probenecid or probenecid analogs have antiviral properties and should be suitable for use in the prevention and/or treatment of multiple families of viruses.

The compounds disclosed herein can be formed by modifying an active probenecid or probenecid analog to include one or more water-soluble moieties. For example, the compound can be prepared by (a) formation of ester, hemiesters, carbonate esters, nitrate esters, amides, hydroxamic acids, carbamates, imines, mannich bases, and enamines of an active probenecid or probenecid analog; (b) functionalizing an active probenecid or probenecid analog with azo, glycoside, peptide, and ether groups; and/or (c) use of polymers, salts, complexes, phosphoramides, acetals, hemiacetals, and ketal forms of an active probenecid or probenecid analog. Additional functional groups that may be used to modify an active probenecid or probenecid analog can be found in Andrejus Korolkovas's, “Essentials of Medicinal Chemistry”, pp. 97-118 and U.S. Pat. No. 8,357,723, which are hereby incorporated by reference in their entirety.

In some forms, the compound is formed by modifying the carboxyl group of probenecid using a water-soluble moiety, such as a carbonate, carbamate, imine, ether, ester, amide, or phosphate. The addition of such water-soluble moieties can effectively diminish the ability of these compounds to cross certain biological membranes, such as those associated with the blood-brain barrier or the blood-placental barrier. In some forms, these compounds show reduced biological membrane crossing rate as compared to the biological membrane crossing rate of probenecid not attached to the water-soluble oligomer. Additionally, compared to probenecid administered in its free form, the prodrugs disclosed herein can alter the pharmacokinetics of probenecid, improve the stability and solubility of probenecid, decrease the toxicity of probenecid, increase the specificity of probenecid, and/or increase the duration of the pharmacological effect of probenecid.

Pharmaceutical compositions and formulations containing the prodrugs are also disclosed.

### 1. Prodrug Compounds

The compound can have the structure of Formula I:

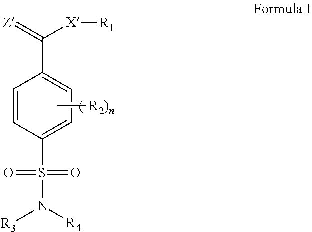

where: (a) Z′ can be O, NR₅, or S; (b) X′ can be absent, O, NR₅, or S; (c) R₁ can be hydrogen, a substituted or unsubstituted alkyl, a substituted or unsubstituted alkenyl, a substituted or unsubstituted alkynyl, a substituted or unsubstituted aryl, a substituted or unsubstituted heteroaryl, a substituted or unsubstituted polyaryl, a substituted or unsubstituted polyheteroaryl, an azo, an alkoxy, a polyether, a thiol, a sulfanimine, an amino, a carbonate, an ester, an amide, a carbamate, an imine, a substituted or unsubstituted carbonyl, a hydroxyl, a polyol, a phosphonyl, sulfinyl, a sulfonamide, a nitro, a cyano, a lipid, a peptide, a cholesterol, a phytosterol, a glycoside, or a glucuronide; (d) n can be an integer from 0 to 4; (e) each R₂ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted aralkyl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen; (f) R₃-R₅ can be independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol; and (g) the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

In some forms of Formula I, R₁ can be hydrogen, a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, a substituted or unsubstituted heteropolyaryl, a polyol, a polyalkylene glycol, a lipid, a peptide, a cholesterol, a phytosterol, a glucuronide,

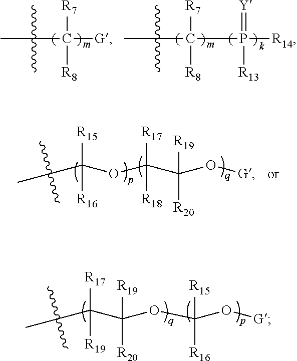

where (a) G′ can be hydrogen, a lipid, a peptide, a cholesterol, a phytosterol, a glycoside, a glucuronide,

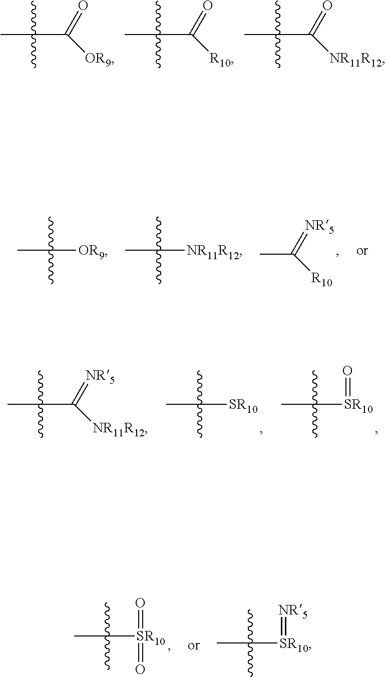

and R₉-R₁₂ can be independently hydrogen, a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, an alkoxy, a di-alkyl amino, or a halogen; R′₅ can be independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol, such as a hydrogen or a substituted or unsubstituted C₁-C₆ alkyl (e.g., an unsubstituted C₁-C₆ linear or branched alkyl, an unsubstituted C₁-C₆ cycloalkyl, an unsubstituted C₁-C₄ linear or branched alkyl, an unsubstituted C₁-C₄ cycloalkyl, an unsubstituted C₁-C₃ linear or branched alkyl, an unsubstituted C₁-C₃ cycloalkyl, etc.); (b) m, k, p, and q can be independently an integer from 0 to 20, from 0 to 18, from 0 to 16, from 0 to 14, from 0 to 12, from 0 to 10, from 0 to 8, from 0 to 6, from 0 to 4, from 0 to 3, or from 0 to 2, such as 0 or 1; (c) each Y′ can be independently O or S; (d) each occurrence of R₇, R₈, and R₁₅-R₂₀ can be independently hydrogen, a substituted or unsubstituted C₁-C₂₀ alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen, or R₇ and R₈ together, R₁₅ and R₁₆ together, and/or R₁₇ and R₁₈ together, with the carbon atom to which they are attached, form a C₁-C₂₀ cycloalkyl, or when X′ is NR₅, m is not 0, at least one of p and q is not 0, then (i) R₇ is hydrogen and R₈ is a substituted or unsubstituted C₁-C₂₀ alkyl that form a ring together with R₅ that includes the adjoining N and C atoms, (ii) R₁₅ is hydrogen and R₁₆ is a substituted or unsubstituted C₁-C₂₀ alkyl that form a ring together with R₅ that includes the adjoining N and C atoms, and/or (iii) R₁₇ is hydrogen and R₁₈ is a substituted or unsubstituted C₁-C₂₀ alkyl that form a ring together with R₅ that includes the adjoining N and C atoms; and (e) R₁₃ and R₁₄ can be independently hydrogen, a substituted or unsubstituted C₁-C₂₀ alkyl, or an alkoxy.

In some forms of Formula I, R₁ can be an unsubstituted C₁-C₂₀ linear or branched alkyl, an unsubstituted C₃-C₂₀ cycloalkyl, a C₁-C₂₀ haloalkyl, an unsubstituted aryl, an unsubstituted polyaryl, an unsubstituted heteroaryl, an unsubstituted heteropolyaryl, a polyalkylene glycol, a lipid, a peptide, a cholesterol, a phytosterol, a glucuronide,

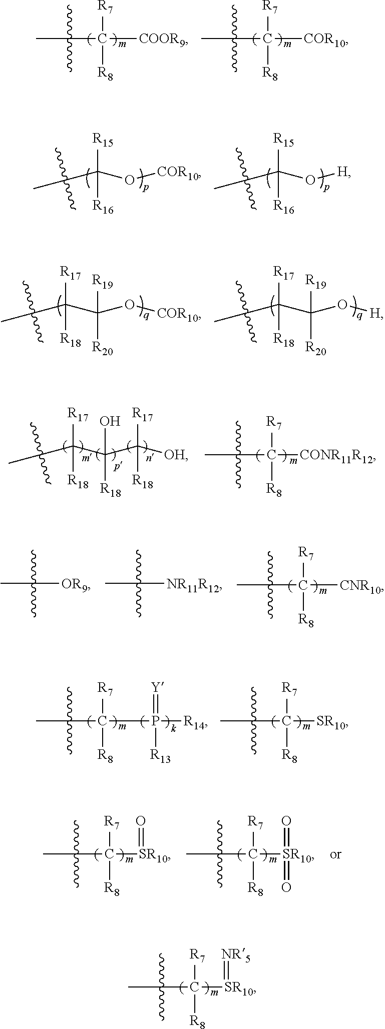

where m, m′, p′, and n′ can be independently an integer from 0 to 10, from 0 to 8, from 0 to 6, from 0 to 4, from 0 to 3, from 0 to 2, or 0 or 1, p can be an integer from 1 to 6, from 1 to 4, from 1 to 3, or 1 or 2, and k can be an integer from 1 to 6, from 1 to 4, or from 1 to 3.

In some forms of Formula I, Z′ can be O. In some forms of Formula I, Z′ can be S. In some forms of Formula I, Z′ can be NR₅. In some forms of Formula I, X′ can be absent, O, or S. In some forms of Formula I, X′ can be absent. In some forms of Formula I, X′ can be O. In some forms of Formula I, X′ can be S. In some forms of Formula I, Z′ can be O and X′ can be O. In some forms of Formula I, Z′ can be O and X′ can be S. In some forms of Formula I, Z′ can be O and X′ can be absent. In some forms of Formula I, Z′ can be S and X′ can be O. In some forms of Formula I, Z′ can be S and X′ can be S. In some forms of Formula I, Z′ can be S and X′ can be absent. In some forms of Formula I, Z′ can be NR₅ and X′ can be absent.

In some forms of Formula I, Z′ can be O, X′ can be absent or O, and R₁ can be a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, a substituted or unsubstituted heteropolyaryl, a polyalkylene glycol, a lipid, a peptide, a cholesterol, a phytosterol, a glycoside, or a glucuronide.

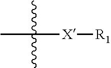

In some forms of Formula I, Z′ can be O or NR₅ and can be

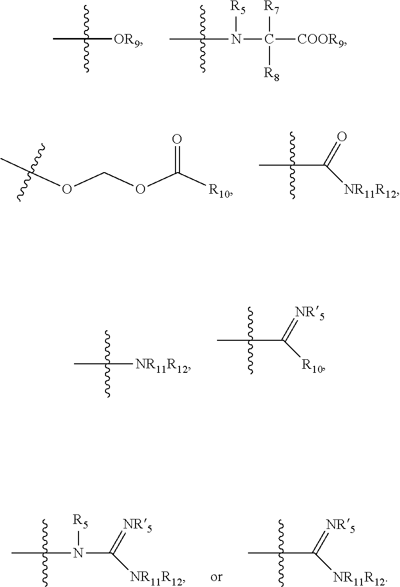

In some forms of Formula I, Z′ can be O, X′ can be O, and R₁ can be

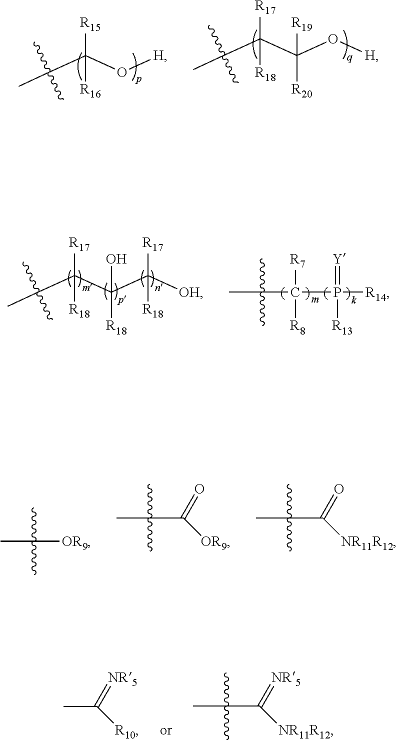

In some forms of Formula I, Z′ can be O, X′ can be S, and R₁ can be

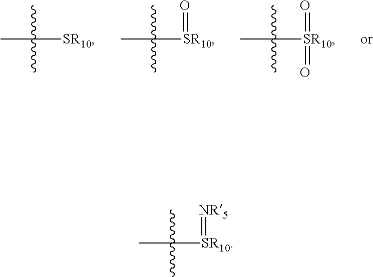

In some forms of Formula I, Z′ can be O, X′ can be O, and R₁ can be

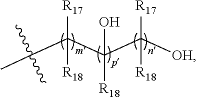

where m′ and n′ are independently an integer from 0 to 6, from 0 to 5, from 0 to 4, from 0 to 3, from 1 to 3, such as 1 or 2; p′ is an integer from 1 to 10, from 1 to 8, from 1 to 6, from 1 to 4, or from 1 to 3; each occurrence of R₁₇ and R₁₈ are independently hydrogen, hydroxyl, —SH, —(CH₂)₁₋₆NR₂₂R₂₃, —(CH₂)₁₋₆OH, —(CH₂)₁₋₆SH, or an unsubstituted C₁-C₁₀ alkyl. In some forms of Formula I, Z′ can be O, X′ can be O, and R₁ can be

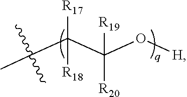

where q is an integer from 1 to 10, from 1 to 8, from 1 to 6, from 1 to 4, or from 1 to 3; each occurrence of R₁₇-R₂₀ are independently hydrogen, hydroxyl, —SH, —(CH₂)₁₋₆NR₂₂R₂₃, —(CH₂)₁₋₆OH, —(CH₂)₁₋₆SH, or an unsubstituted C₁-C₁₀ alkyl. In these forms of Formula I, R₂ can be hydrogen, hydroxyl, —SH, —(CH₂)₁₋₆NR₂₂R₂₃, —(CH₂)₁₋₆OH, —(CH₂)₁₋₆SH, or an unsubstituted C₁-C₁₀ alkyl, such as hydrogen; and R₃ and R₄ can be independently a an unsubstituted C₁-C₁₀ alkyl, such as an unsubstituted methyl, ethyl, propyl, butyl, pentyl, or hexyl, for example, an unsubstituted propyl.

In any one of the forms of Formula I described above, each occurrence of R₇, R₈, and R₁₅-R₂₀ can be independently hydrogen, a substituted or unsubstituted C₁-C₂₀ alkyl, —(CH₂)₁₋₆NR₂₂R₂₃, —(CH₂)₁₋₆OH, a substituted or unsubstituted aralkyl, —(CH₂)₁₋₆SH, —(CH₂)₁₋₆S(O)₀₋₂CH₃, —(CH₂)₁₋₆NHC(═NH)NH₂, -(1H-indol-3-yl) methyl, -(1H-imidazol-4-yl)methyl, —(CH₂)₀₋₆COOR₂₁, —(CH₂)₀₋₆CONR₂₂R₂₃, a substituted or unsubstituted aryl, an aryl-C₁₋₃ alkyl, CH₂-indol-3-yl, —(CH₂)₁₋₆SCH₃, —CH₂-imidazol-4-yl, CH(OH)(CH₂)₀₋₅CH₃, —CH₂((4′-OH)-Ph), where R₂₁-R₂₃ can be independently hydrogen or an unsubstituted C₁₋₆ alkyl. In some forms of Formula I, each occurrence of R₇, R₈, and R₁₅-R₂₀ can be independently hydrogen, an unsubstituted C₁-C₂₀ alkyl, —(CH₂)₁₋₆NR₂₂R₂₃, —(CH₂)₁₋₆OH, an unsubstituted aralkyl, —(CH₂)₁₋₆SH, —(CH₂)₁₋₆S(O)₀₋₂CH₃, —(CH₂)₁₋₆NHC(═NH)NH₂, -(1H-indol-3-yl) methyl, -(1H-imidazol-4-yl)methyl, —(CH₂)₀₋₆COOR₂₁, —(CH₂)₀₋₆CONR₂₂R₂₃, an unsubstituted aryl, an aryl-C₁₋₃ alkyl, CH₂-indol-3-yl, —(CH₂)₁₋₆SCH₃, —CH₂-imidazol-4-yl, CH(OH)(CH₂)₀₋₅CH₃, —CH₂((4′-OH)-Ph), where R₂₁-R₂₃ can be independently hydrogen or an unsubstituted C₁₋₆ alkyl.

In any one of the forms of Formula I described above, R₃ and R₄ can be independently hydrogen, a substituted or unsubstituted alkyl, a substituted or unsubstituted aryl, a substituted or unsubstituted hetercyclyl, a substituted or unsubstituted heteroaryl, an alkoxy, an amino, or an imine, preferably R₃ and R₄ are independently hydrogen or a substituted or unsubstituted C₁-C₂₀ alkyl.

In any one of the forms of Formula I described above, R₅ and/or R′₅ can be hydrogen or a substituted or unsubstituted C₁-C₂₀ alkyl, such as an unsubstituted C₁-C₂₀ linear or branched alkyl or an unsubstituted C₁-C₂₀ cycloalkyl.

In any one of the forms of Formula I described above, when substituents are present, the substituents can be independently an unsubstituted C₁-C₆ alkyl, a C₁-C₆ alkyl substituted with unsubstituted C₁₋₆ alkyl, an unsubstituted C₁-C₆ heteroalkyl, a C₁-C₆ heteroalkyl substituted with unsubstituted C₁₋₆ alkyl, an unsubstituted C₂-C₆ alkenyl, an unsubstituted C₂-C₆ alkynyl, an unsubstituted aryl, an unsubstituted heteroaryl, an unsubstituted C₁-C₆ alkoxy, —(CH₂)₁₋₆CO₂R₂₁, a halogen, C₁-C₆ haloalkyl, —NR₂₂R₂₃, C₁₋₆ acylamino, —NHSO₂C₁₋₆ alkyl, —SO₂NR₂₂R₂₃, —SO₂C₁₋₆ alkyl, —COOR₂₁, —CONR₂₂R₂₃, nitro, cyano, hydroxide, thiol, or an aryl or heteroaryl substituted with unsubstituted C₁_₅ alkyl, an alkoxy, a di(C₁₋₆ alkyl)-amino, a fluoro, or an unsubstituted C₃-C₆ cycloalkyl.

In some forms of Formula I, when R₁ is a peptide, the peptide can be any one of the peptides shown in Table 2.

TABLE 2
Cell penetrating peptides

| Peptide | Sequence | Length | Origin | References |
|---|---|---|---|---|
| Cationic | | | | |
| CPPs | | | | |
| TAT | RKKRRQRRR | 9 | Protein | Baoum et al., Int J Pharm. |
| | (SEQ ID NO: 6) | | derived | 2012 May 1; 427(1):134-42 |
| R8 | RRRRRRRR | 8 | Synthetic | Chu et al., Nanomedicine. |
| | (SEQ ID NO: 7) | | | 2015 February; 11(2):435- |
| | | | | 462015 |
| DPV3 | RKKRRRESRKK | 16 | Protein | De coupade et al., Biochem |
| | RRRES (SEQ ID | | derived | J. 2005 Sep. 1; 390(Pt |
| | NO: 8) | | | 2):407-18. 2005 |
| DPV6 | GRPRESGKKRK | 17 | Protein | De coupade et al., Biochem |
| | RKRLKP (SEQ ID | | derived | J. 2005 Sep. 1; 390(Pt |
| | NO: 9) | | | 2):407-18. 2005 |
| Penetratin | RQIKIWFQNRR | 16 | Protein | Nielsen et al., J Control |
| | MKWKK (SEQ | | derived | Release, 2014 Sep. |
| | ID NO: 10) | | | 10; 189:19-24. 2014 |
| R9-TAT | GRRRRRRRRRP | 13 | Protein | Futaki et al., J Biol Chem. |
| | PQ (SEQ ID | | derived | 2001 Feb. 23; 276(8):5836- |
| | NO: 11) | | | 40. 2001 |
| Amphipathic | | | | |
| CPPs | | | | |
| pVEC | LLIILRRRIRKQA | 18 | Protein | Eggimann et al., Chem |
| | HAHSK (SEQ ID | | derived | Commun (Camb). 2014 Jul. |
| | NO: 12) | | | 14; 50(55):7254-7. 2014 |
| ARF | RVRVFVVHIPRL | 13 | Protein | Johansson et al., Mol Ther. |
| (19-31) | T (SEQ ID NO: 13) | | derived | 2008 January; 16(1):115-23. |
| | | | | 2008 |
| MPG | GALFLGFLGAA | 27 | Chimeric | Simeoni, Nucleic Acids |
| | GSTMGAWSQPK | | | Res. 2003 Jun. |
| | KKRVK (SEQ ID | | | 1; 31(11):2717-24 2003 |
| | NO: 14) | | | |
| MAP | KLALKLALKAL | 18 | Synthetic | Wada et al., Bioorg Med |
| | KAALKLA (SEQ | | | Chem. 2013 Dec. |
| | ID NO: 15) | | | 15; 21(24):7669-73 2013 |
| Trans- | GWTLNSAGYLL | 27 | Protein | Pae et al., J Control |
| portan | GKINLKALAAL | | derived | Release. 2014 Oct. |
| | AKKIL (SEQ ID | | | 28; 192:103-13. 2014 |
| | NO: 16) | | | |
| Hydrophobic | | | | |
| CPPs | | | | |
| Bip4 | VSALK (SEQ ID | 5 | Protein | Gomez et al., |
| | NO: 17) | | derived | Pharmaceuticals (Basel). |
| | | | | 2010 December; 3(12):3594- |
| | | | | 3613. 2010 |
| C105Y | CSIPPEVKFNPF | 16 | Protein | Rhee and Davis, J Biol |
| | VYLI (SEQ ID | | derived | Chem., 2006 Jan. |
| | NO: 18) | | | 13; 281(2):1233-40 2006 |
| Melittin | GIGAVLKVLTT | 26 | Protein | Hou et al., Biomaterials. |
| | GLPALISWIKRK | | derived | 2013 April; 34(12):3110-9 |
| | RQQ (SEQ ID | | | |
| | NO: 19) | | | |
| gH625 | HGLASTLTRWA | 20 | Protein | Galdiero et al., Biochim |
| | HYNALIRAF | | derived | Biophys Acta. 2015 |
| | (SEQ ID NO: 20) | | | January; 1848(1 Pt A):16-25. |
| | | | | 2015 |

In some forms of Formula I, the compound is not any one of the compounds in Table 1 above.

Examples of probenecid prodrugs are shown below:

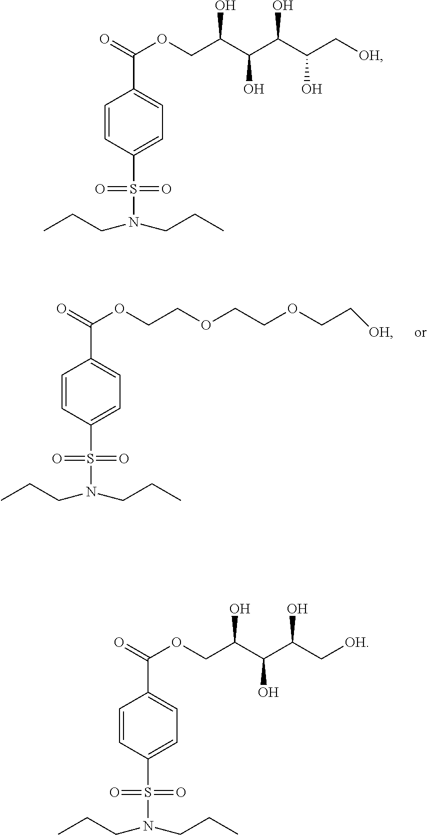 [SMILES: CCCN(CCC)S(=O)(=O)c1ccc(C(=O)OC[C@@H](O)[C@H](O)[C@@H](O)[C@@H](O)CO)cc1 ; CCCN(CCC)S(=O)(=O)c1ccc(C(=O)OCCOCCOCCO)cc1 ; "or" ; CCCN(CCC)S(=O)(=O)c1ccc(C(=O)OC[C@@H](O)[C@H](O)[C@@H](O)CO)cc1]

The prodrugs may contain one or more chiral centers or may otherwise be capable of existing as multiple stereoisomers. These may be pure (single) stereoisomers or mixtures of stereoisomers, such as enantiomers, diastereomers, and enantiomerically or diastereomerically enriched mixtures. The prodrugs may be capable of existing as geometric isomers. Accordingly, it is to be understood that the disclosed prodrugs include pure geometric isomers or mixtures of geometric isomers.

### 2. Pharmaceutically Acceptable Salts

The compound including prodrugs may be neutral or may be one or more pharmaceutically acceptable salts, crystalline forms, non crystalline forms, hydrates, or solvates, or a combination thereof. References to the compounds including prodrugs may refer to the neutral molecule, and/or those additional forms thereof collectively and individually from the context. Pharmaceutically acceptable salts of the prodrugs include the acid addition and base salts thereof.

Suitable acid addition salts of the prodrugs are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the compounds including prodrugs are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases of the prodrugs may also be formed, for example, hemisulphate and hemicalcium salts.

Typically, the metabolites, analogs, and prodrugs can, upon metabolism thereof by a subject, prevent and/or treat a virus when administered in an effective amount as discussed herein. For example, in some embodiments, the prodrug can, upon metabolism thereof by a subject, reduce viral replication.

### B. Formulations

Probenecid, metabolites, analogs, and prodrugs thereof, and pharmaceutically acceptable salts thereof can be formulated in a composition, for example, a pharmaceutical composition or animal feed or water. Pharmaceutical compositions can be for administration by parenteral (intramuscular, intraperitoneal, intravenous (IV) or subcutaneous injection), enteral, topical, transdermal (either passively or using iontophoresis or electroporation), transmucosal (nasal, pulmonary, vaginal, rectal, or sublingual) routes of administration or using bioerodible inserts and can be formulated in dosage forms appropriate for each route of administration. Formulations can also be administered via ocular routes, e.g., topical, local ocular (ie, subconjunctival, retrobulbar, intracameral, intravitreal), and systemic delivery to the eyes. See, e.g., Whelan, Merck Manual, Veterinary Manual, “Systemic Pharmacotherapeutics Of The Eye,” (2022). For example, in some embodiments, the compounds are administered in eye drops.

The compositions can be administered systemically.

The compositions can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage forms: tablets”, eds. Liberman et. al. (New York, Marcel Dekker, Inc., 1989), “Remington—The science and practice of pharmacy”, 21st ed., Lippincott Williams & Wilkins, Baltimore, M D, 2006, and “Ansel's Pharmaceutical dosage forms and drug delivery systems”, 11^{th} Edition, Loyd Allen., (Media, PA: Williams and Wilkins, 2017) which provides information on carriers, materials, equipment and process for preparing tablets and capsules and delayed release dosage forms of tablets, capsules, and granules.

The simulation studies show that while safe, preferably probenecid should be administered in relatively large doses to maintain steady state concentration in the plasma and to provide a therapeutic level of concentration. To decrease the dosage and to attain a steady release of drug over several days, delivery vehicles can be employed for delivering probenecid as well as probenecid metabolites, analogs, and prodrugs to achieve the same or improved effect with a smaller dose and/or less frequent administration. Smaller doses and/or less frequent administration may also increase patient compliance.

Thus, the compounds can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by, or bound to, a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before all of the first agent is released. In still other embodiments, both agents are released at the same time over the same period of time or over different periods of time.

Cyclodextrins can be employed for manufacturing an aqueous based formulation of drugs like probenecid with low or no water solubility. The formulation comprises cyclodextrins and probenecid with and a sufficient amount of water to solubilize the cyclodextrins, wherein probenecid is driven into the cyclodextrins. For example, an aqueous composition can be heated to a temperature which is less than the decomposition point but above the melting point of the compound; wherein the compound and cyclodextrins are mixed in a ratio of from about 1:100 to about 1:10 respectively, on a percent weight basis. In a specific embodiment, the cyclodextrin is hydroxypropyl β-cyclodextrin.

Liposomes are water-in-oil-in-water (w/o/w) emulsions with closed bilayer membranes that contain an entrapped aqueous volume. Liposomes encapsulate both hydrophilic and hydrophobic molecules. Liposomes are of two types: multilamellar vesicles (MLVs) and large unilamellar vesicles (LUVs). Unilamellar and multilamellar liposomes and can be prepared by standard methods and from commercially available phospholipids such as phosphatidylcholine and phosphatidylethanolamine. The drug-encapsulating liposomes can be used as a liquid suspension (for intravenous injection) or attached to the surface of an implant. Attachment can be achieved by covalent tethering using click chemistry or by embedding in a biocompatible hydrogel. In an exemplary embodiments, a 65:10:25 molar ratio DMPC: DSPG: Cholesterol is used to form liposomes, though other components and/or ratios are also contemplated. Exosomal formulations can be prepared by mixing the compound with EL-4 exosomes in PBS.

### 1. Oral Immediate Release Formulations

Suitable oral dosage forms include tablets, capsules, solutions, suspensions, syrups, and lozenges. Thus, the composition can be formulated as a solid or liquid. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

### 2. Extended Release Dosage Forms

The extended release formulations are generally prepared as diffusion or osmotic systems, for example, as described in “Remington—The science and practice of pharmacy” (21st ed., Lippincott Williams & Wilkins, Baltimore, M D, 2006). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

### 3. Delayed Release Dosage Forms

Delayed release formulations are created by coating a solid dosage form with a film of a polymer that is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITS®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### Methods of Manufacturing

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel's Pharmaceutical Dosage Forms and Drug Delivery Systems, 11th Ed. (Media, PA: Williams & Wilkins, 2017).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### 4. Formulations for Mucosal and Pulmonary Administration

The probenecid, metabolites, analogs, and prodrugs thereof, and pharmaceutical compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In some embodiments, the compound is formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption.

Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

In some embodiments, the composition is formulated as an aerosol. The term aerosol as used herein refers to any preparation of a fine mist of particles, which can be in solution or a suspension, whether or not it is produced using a propellant. Aerosols can be produced using standard techniques, such as ultrasonication or high-pressure treatment.

Probenecid has been used to reduce nephrotoxicity of cidofovir when administered intravenously. Delivering cidofovir directly to the respiratory tract was also shown to be an effective prophylactic strategy that maximizes the tissue concentration at the site of initial viral replication, while minimizing its accumulation in the kidneys (Roy, et al., Antimicrob Agents Chemother., 47(9): 2933-2937 (2003)).

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter, and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

Compositions and methods of preparing inhalation-type pharmaceutical compositions including probenecid are described in U.S. Published Application No. 2015/0272870.

Thus, formulations and methods of administering the disclosed compositions to the nasal mucosa and/or the lungs by intranasal delivery, and to the lung by oral inhalation are provided. With respect to intranasal delivery, the formulation and delivery device can be selected and prepared to drive absorption through the nasal mucosa or the lungs. The nasal mucosa is—compared to other mucous membranes—easily accessible and provides a practical entrance portal for small and large molecules (Bitter, et al., “Nasal Drug Delivery in Humans,” in Surber, et al., (eds): Topical Applications and the Mucosa. Curr Probl Dermatol. Basel, Karger, 2011, vol 40, pp 20-35, Pires, et al., J Pharm Pharmaceut Sci., 12(3) 288-311, 2009, and Djupesland, Drug Deliv. and Transl. Res., 3:42-62 (2013) DOI 10.1007/s13346-012-0108-9). Intranasal administration offers a rapid onset of therapeutic effects, reduced first-pass effect, reduced gastrointestinal degradation and lung toxicity, noninvasiveness, essentially painless application, and easy and ready use by patients—particularly suited for children—or by physicians in emergency settings. Flu Mist®, for example, is an exemplary effective nasal influenza vaccine spray.

Numerous delivery devices are available for intranasal administration. Devices vary in accuracy of delivery, dose reproducibility, cost and ease of use. Metered-dose systems provide dose accuracy and reproducibility. Differences also exist in force of delivery, spray patterns and emitted droplet size. The latter being important for drug deposition within the nasal cavity. Parameters can be can be modulated to enhance deposition while limiting the fraction of small particles able to bypass the nose and enter the lungs, or reduce deposition while increasing the fraction of small particles able to bypass the nose and enter the lungs.

The following aspects of nasal anatomy can influence drug delivery. During exhalation the soft palate closes automatically, separating the nasal and oral cavities. Thus, it becomes possible to use smaller particles in a nasal spray and still avoid lung deposition. Additionally, during closure of the soft palate there is a communication pathway between the two nostrils, located behind the walls separating the two passages. Under these circumstances, it is possible for airflow to enter via one nostril and leave by the other. This bidirectional delivery concept combines the two anatomical facts into one fully functional device. The device is inserted into one nostril by a sealing nozzle, and the patient blows into the mouthpiece. The combination of closed soft palate and sealed nozzle creates an airflow which enters one nostril, turns 1800 through the communication pathway and exits through the other nostril (bidirectional flow). Since delivery occurs during exhalation, small particles cannot enter the lungs.

Particle size, flow rate and direction can be tuned for efficient delivery to the nasal mucosa. By adding an exit resistor to give additional control of the input pressure, it is possible to improve distribution to the sinuses and the middle ear. Manipulation of the flow pattern enables delivery to the olfactory region, thereby possibly achieving direct ‘nose-to-brain’ delivery. The 180-degree turn behind the septum will trap particles still airborne, allowing targeted delivery of cargo to the adenoid.

Strategies for enhancing drug absorption via nasal and pulmonary routes are also known in the art and can be utilized in the disclosed formulations and methods of delivery. Such strategies include, for example, use of absorption enhancers such as surfactants, cyclodextrins, protease inhibitors, and tight junction modulators, as well as application of carriers such as liposomes and nanoparticles. See, e.g., Ghadiri, et al., Pharmaceutics, 11(3): 113 (2019).

For example, in a particular embodiment, the disclosed compounds are formulated as an aerosol composition for treating a respiratory disease or disorder including a suspension of the drug particles in a propellant, the process including forming a slurry of a bulking agent such as lactose with a low volatility solvent like ethanol and reducing the mass median diameter particle size of the bulking agent to less than one micron by subjecting the slurry to high pressure homogenation, and thereafter mixing the resulting slurry with other components of the aerosol formulation, wherein the other components of the aerosol formulation include a drug and a propellant, and wherein the mass median diameter particle size of the drug is equal to or greater than 1 micron. The aerosol formulation can be administered either nasally or orally.

### 5. Formulations for Parenteral Administration

Probenecid, metabolites, analogs, and prodrugs thereof, and pharmaceutical compositions thereof can be administered in an aqueous solution, by parenteral injection or infusion. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions. In some embodiments, the formulation is a long-acting parenteral formulation, with or without the use of implantable devices and/or delivery vehicles. These can include, but are not limited to, micro-encapsulation, oil-based formulations, nanoparticle or nanocrystal formulations, and hydrogel formulations.

### 6. Topical and Transdermal Formulations

Transdermal formulations may also be prepared. These will typically be gels, ointments, lotions, sprays, or patches, all of which can be prepared using standard technology. Transdermal formulations can include penetration enhancers.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) composed of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alkylene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some embodiments, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some embodiments, the penetration enhancer is, or includes, an alcohol such ethanol, or others disclosed herein or known in the art.

Delivery of drugs by the transdermal route has been known for many years. Advantages of a transdermal drug delivery compared to other types of medication delivery such as oral, intravenous, intramuscular, etc., include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

### 7. Animal Feed, Water, and Milk

Probenecid, metabolites, analogs, and prodrugs thereof, and pharmaceutically acceptable salts thereof can be formulated in animal feed, supplements, drinking water, and/or milk. Animal feed can include commercial livestock feed, and the like.

Exemplary animal feeds include chicken feeds, including (i) starter diets, grower diets and/or finisher diets, particular for a meat-type chicken such as broiler chicken, or (ii) for egg-laying chicken such as a pullet or hen, or (iii) for breeder chickens. Also included are feeds for other poultry, such as a turkey, geese, quail, pheasant, or ducks, or livestock, such as cattle, sheep, goats or swine, alpaca, banteng, bison, camel, cat, deer, dog, donkey, gayal, guinea pig, horse, llama, mule, rabbit, reindeer, water buffalo, yak, although the skilled person will appreciate that other feeds for animals, including zoo animals, captive animals, game animals, domestic animals such as cats and dogs, rodents (such as mice, rats, guinnea pigs, hamsters), and horses, are also provided, as well as any other domestic, wild and farmed animals, including mammals and birds. For example, in some embodiments, the probenecid, metabolite, analog, or prodrug thereof, or pharmaceutically acceptable salt thereof is formulated as a part of chicken feed. Chicken feed diets generally contain crude proteins, fats, sugars, amino acids, minerals, starch, and vitamins. There are many ingredients available, see, e.g., Commercial Poultry Nutrition, 3rd Edition, University books, Steven Leeson, John D. Summers, P.O. Box 1326 Guelph, Ontario, Canada N1H 6N8 (2005), the entire contents of which is specifically incorporated by reference herein in its entirety. Chapter 2 describes the advantages and disadvantages of the common ingredients in such diets in detail. The major ingredients delivering energy in diets are corn, wheat, soybean, soy oil and amino acids. Corn can be a major contributor of metabolizable energy. Thus, in some embodiments, the probenecid, metabolite, analog, or prodrug thereof, or pharmaceutically acceptable salt thereof is formulated is formulated with one or more of corn, soybean, sorghum, vegetable fat, molasses, vitamins, minerals, amino acids, salt, phosphate, calcium, or a combination thereof alone or in further combination with other materials. Chickens used in optimized commercial broiler production are typically fed different diets depending upon their age. For example, chickens for broiler production may be raised using three diets. These diets are typically called a “starter”, “grower” and “finisher”. “Pre-starter” diets are also possible. The disclosed compounds can be added to any of the forgoing diets.

Similarly, in some embodiments, the probenecid, metabolite, analog, or prodrug thereof, or pharmaceutically acceptable salt thereof is formulated as a part of swine feed. Swine feed can be formed of one or more of grains (i.e., corn, wheat, barley, oats), oilseed meals (i.e., soybean meal, cottonseed meal, flaxseed meal, canola meal, sunflower meal), byproducts (i.e., wheat middlings, wheat bran, rice bran, corn distiller dried grains, brewers grains, corn gluten meal, corn gluten feed, molasses, rice mill byproduct), oils (i.e., corn oil, flax oil, soy oil, palm oil, animal fat, restaurant grease, and blends thereof), vitamins and minerals, amino acids, antioxidants, tocochromanols, tocopherols, salt, coccidostats and/or antibiotics, enzymes (i.e., phytase, xylanase), and other feed additives.

Feed additives and supplement may include, for example, macro minerals, which include those selected from the group consisting of calcium, phosphorus, magnesium, sodium, potassium and chloride; trace Minerals, including zinc and/or selenium; and/or added vitamins, which include those selected from the group consisting of vitamin A, nicotinic acid, pantothenic acid, pyridoxine (B6) and biotin in maize and wheat-based feed. Additionally, there is a basic requirement of broiler chickens for vitamin E at 10-15 mg/kg. The need for extra supplementation with vitamin E will depend on the level and type of fat in the diet, on the level of selenium and on the presence of pro- and anti-oxidants. Heat treatment of feeds can result in the destruction of up to 20% of vitamin E. Choline may also be given in a complete feed.

Non-nutritive feed additives may also be included. Enzymes are routinely used in poultry feeds to improve digestibility of feed ingredients. In general, feed enzymes are available that act on carbohydrates, plant bound minerals and proteins. Non Starch Polysaccharide (NSP) enzymes are economically beneficial in wheat-based feeds. These enzymes will also allow greater flexibility in the levels of barley to be included in the ration. Phytase enzymes can be used to enhance phytate phosphorus utilization. Protease enzymes can be included to act upon vegetable products. Carbohydrase enzymes can be added, and may provide beneficial responses when used in maize-soya diets. When adding enzymes before heat processing of broiler feeds, there is the potential for a loss in enzyme activity. This may be avoided by spraying enzymes on to the feed at the end of processing.

Additional medicinal and/or prophylactic may be added. A wide range of medicinal products, e.g., coccidiostats and antibiotics, may be administered through the feed. Antibiotic Growth Promoters/Digestion Enhancers can be included and can, for example, provide a mode of action involving modification of the gut microflora, with consequential benefits in nutrient utilization.

Prebiotics can be added, and refer to a group of substances which stimulate the growth of beneficial microorganisms, at the expense of harmful, micro-organisms. Oligosaccharides form the largest group of these products at present.

Probiotics can be added to introduce live micro-organisms into the digestive tract to assist the establishment of a stable and beneficial microflora. The objective is to provide the gut with positive, non-pathogenic micro-organisms which will then prevent colonization with pathogenic micro-organisms by competitive exclusion.

Organic Acids may be added. Organic acid products can be used to reduce bacterial contamination of the feed (e.g. after heat treatment) and can also encourage beneficial microflora to develop in the digestive tract of the bird.

Absorbents are used specifically to absorb mycotoxins. They may also have a beneficial effect on general bird health and nutrient absorption. There are a range of products available for use as absorbents, including various clays and charcoal.

Antioxidants can provide important protection against nutrient loss in broiler feeds. Some feed ingredients e.g. fish meal and fats, can be protected. Vitamin premixes should be protected by an antioxidant unless optimum storage times and conditions are provided. Additional antioxidants may be added to the final feed where prolonged storage or inadequate storage conditions are unavoidable.

Anti-Mold Agents can be added. For example, mold inhibitors may be added to feed ingredients, which have become contaminated, or to finished rations to reduce growth of fungi and production of mycotoxins.

Pelleting agents can be added, and are used to improve pellet hardness. Some examples of pellet binders are hemicellulose, bentonite and guar gum.

Other products of possible use in feed and supplement production include essential oils, nucleotides, glucans and specialized plant extracts. In areas of the world where its use is permitted, formaldehyde can be used to treat/preserve feed.

### III. Methods of Treatment

Methods of use are also provided. The methods typically include administering to a subject in need thereof, an effective amount of probenecid or a metabolite, analog, or prodrug thereof, or a pharmaceutically acceptable salt thereof, or composition or formulation formed therefrom.

A. Methods of Treating and/or Preventing Viral Infections

Methods of treating a viral infection in subject in need thereof are provided. Thus, method of treating and/or preventing a viral infection and/or symptoms associate therewith are provided.

In some embodiments, the virus can be one that causes a respiratory disease or illness or a non-respiratory disease or illness. Thus, methods of treating a respiratory disease or illness, particularly in a subject infected with a virus are also provided.

The methods can be prophylactic. Thus, methods of preventing viral infection and/or respiratory or non-respiratory diseases and illnesses particularly from infection with a virus are also provided.

The methods can include administering to a subject an effective amount of probenecid, a metabolite, analog, or prodrug thereof, or a pharmaceutically acceptable salt thereof to reduce viral replication, infection, or a combination thereof. In some embodiments, the amount is effective to reduce virus titer in the subject, reduce the host cell from assembling virus, reduce and/or limit hyper-inflammation associated with infection, and/or one or more severe respiratory symptoms.

In some embodiments, the subject has been, or will be, exposed to the virus. In some embodiments, the subject has been exposed to the virus or is experiencing an active viral infection.

In some embodiments, a viral infection is detected by PCR test designed to detect viral DNA or RNA in a sample from the subject, for example, a nasal swab, throat swab, saliva, or other bodily fluid; or a serological or immunodiagnostic test designed to detect antibodies, typically in a blood sample from the subject, that the body's immune system has produced in response to the infection.

The compositions can also be administered prophylactically to, for example, reduce or prevent the effects of future exposure to virus and the infection that may associated therewith. Thus, in some embodiments, the subject has not been exposed to the virus and/or is not yet experiencing an active viral infection. In some embodiments, the subject is a healthy subject.

In some embodiments, the subject has been in close contact with a subject that has tested positive for the virus. Such a subject may or may not be exhibiting one or more symptoms of an infection. Close contact may be or include, for example, being within 6 feet (or 2 meters) of someone who is infected for a total of 15 minutes or more, providing care at home to someone who is infected, having direct physical contact with the person (hugged or kissed them) who is infected, sharing eating or drinking utensils with an infected person, having been sneezed on, coughed on, or otherwise by contacted by respiratory droplets from an infected person.

In some embodiments, the subject was identified in contact-tracing as having been exposed to the virus, or one or more subjects infected therewith.

In some embodiments, the subject will be exposed to the virus. An exemplary subject is a healthcare worker that will treat infected people.

In some embodiments, treatment begins 1, 2, 3, 4, 5, or more hours, days, or weeks prior to or after exposure to the virus.

In some embodiments, the probenecid, a metabolite or analog or prodrug thereof, or a pharmaceutically acceptable salt thereof is administered in an effective amount to reduce or prevent one or more symptoms of a viral infection. Symptoms include those of an acute respiratory illness, for example, fever, congestion in the nasal sinuses and/or lungs, runny or stuffy nose, cough, sneezing, sore throat, body aches, fatigue, shortness of breath, chest tightness, and wheezing when exhaling. Exemplary viruses and particular symptoms associated with infection thereby are discussed in more detail below.

In some embodiments, the subject does not have gout, need prolonged penicillin (or other antibiotic) serum levels, pelvic inflammatory disease, or gonorrhea.

### 1. Exemplary Viruses

The disclosed compounds, compositions, and method of use can be used to prevent and therapeutically treat infection by one or more virus.

In some embodiments, the virus is an RNA virus. In some embodiments, the virus is a negative strand RNA virus. In some embodiments, the virus is a DNA virus. In some embodiments, the virus is not a DNA virus.

In preferred embodiments, the viruses have an RNA genome, e.g., having a negative-sense or positive-sense genome made of ribonucleic acid. In some embodiments, the viruses encode an RNA-dependent RNA polymerase (RdRp). In some embodiments, the viruses are members of the kingdom Orthornavirae,

Viruses can belong to, for example, the families Adenoviridae, Papoviridae, Herpesviridae, Poxviridae, Anelloviridae, Pleolipoviridae, Reoviridae, Picornaviridae, Caliciviridae, Togaviridae, Arenaviridae, Flaviviridae, Orthomyxoviridae, Paramyxoviridae, Bunyaviridae, Rhabdoviridae, Filoviridae, Coronaviridae, Astroviridae, Bornaviridae, Arteriviridae or Hepeviridae.

For example, in some embodiments, the virus is from a negative-sense RNA virus family such as Arenaviridae, Bunyaviridae, Filovirida, Nymaviridae, Orthmyxoviridae, Paramyxoviridae, Pneumoviridae, or Rhabdoviridae; or a positive strand family such as Arteriviridae, Astroviridae, Caliciviridae, Coronaviridae, Flaviviridae, Hepeviridae/Nodaviridae, Picornaviridae, orTogaviridae.

Host gene pathway analysis indicate that replication of some virus can intersect OAT activity and that OATs may be needed for transport of viral constituents needed for viral replication using a similar process of OAT-mediated vectorial transport as for sodium and chloride ions across the airway lumen (Zhang, et al., J Virol 76, 5654-5666, doi:10.1128/jvi.76.11.5654-5666.2002 (2002), Chen, et al., Am J Respir Cell Mol Biol 40, 588-600, doi:10.1165/rcmb.2008-0034OC (2009)). In some embodiments, the virus is one that utilizes an organic anion transporter. In some embodiments, the transporter is a SLC22 family member, see, e.g., Engelhart, et al., Int. J. Mol. Sci., 21(5), 1791 (2020); doi.org/10.3390/ijms21051791, which is specifically incorporated by reference herein in its entirety. In some embodiments, the transporter is an OAT selected from OAT1, OAT2, OAT3, OAT4, OAT5, OAT6, OAT7, rOAT8, OAT9, OAT10, and/or URAT1. In some embodiments, the probenecid, metabolite, analog, or prodrug, or pharmaceutically acceptable salt thereof is effective to reduce or inhibit the activity of a transporter, such as one or more of the foregoing.

In some embodiments, the compositions are used as a prophylactic or therapeutic pan-antiviral, preventing or treating infection by two or more viruses.

Examples of viruses infection by which can be prevented and/or treated include, but are not limited to, influenza viruses, such as influenza virus A, influenza virus B, influenza virus C, respiratory syncytial virus (RSV), human metapneumovirus, coronaviruses, measles virus, parainfluenza virus, mumps virus, Zika virus, dengue virus, yellow fever virus, Japanese encephalitis, Ebola virus, hantaviruses, Lassa fever virus, and West Nile virus. Viral infections include viral infections of the liver. Examples of viruses causing infections of the liver include, but are not limited to, Hepatitis A virus, Hepatitis B virus, and Hepatitis C virus. Some such viruses are discussed in more detail below.

In some embodiments the virus is a respiratory virus. Thus, viral infections include viral infections of the respiratory tract.

In some embodiments, the subject is infected with a virus that is the target of the disclosed compounds, compositions, and methods, a second virus that may or may not be treatable with the disclosed compounds, compositions, and methods. In some embodiments, the second virus is human immunodeficiency virus (HIV).

a. Respiratory Syncytial Virus (RSV)

In some embodiments, the virus is respiratory syncytial virus (RSV), also called human respiratory syncytial virus (hRSV) and human orthopneumovirus. RSV is a common, contagious virus that causes infections of the respiratory tract. It is a negative-sense, single-stranded RNA virus, and its name is derived from the large cells known as syncytia that form when infected cells fuse.

RSV is divided into two antigenic subtypes, A and B, based on the reactivity of the F and G surface proteins to monoclonal antibodies. The subtypes tend to circulate simultaneously within local epidemics, although subtype A tends to be more prevalent. Generally, RSV subtype A (RSVA) is thought to be more virulent than RSV subtype B (RSVB), with higher viral loads and faster transmission time.

A working model of RSV transmission is that yearly outbreaks of RSV are the result of variants that grow out of locally evolved clades, not necessarily viruses that have been introduced from distant locations Griffiths, et al., Clinical microbiology reviews, 30(1):277-319 (2017) doi:10.1128/CMR.00010-16. Initially, 5 RSVA clades and 4 RSVB clades were identified, named GA1 to GA5 and GB1 to GB4, respectively. This list of clades has since grown to 16 RSVA clades and 22 RSVB clades. A recent global survey identified GA1, GA2, GA5, and GA7 as the major circulating clades of RSVA worldwide. GA7 is a major circulating clade of RSVA that is found only in the United States. The BA clade of RSVB predominates worldwide. The disclosed compounds, compositions, and methods can be utilized to treat or prevent RSVA, RSVB, or a combination thereof. The disclosed compounds, compositions, and methods can be utilized to treat or prevent any one of more of GA1-GA16, and one or more of GB1-GB22, or any combination thereof.

b. Mumps Virus

Mumps virus, scientific name Mumps orthorubulavirus, is assigned to the genus Orthorubulavirus, in the subfamily Rubulavirinae, family Paramyxoviridae. The mumps virus has one serotype and twelve genotypes. The genotypes can be distinguished based on the F, SH, are HN genes. The SH gene has a degree of variation between genotypes ranging from 5% to 21%, the highest among MuV's genes. The genotypes are named genotypes A to N, excluding E and M, i.e. genotypes A, B, C, D, F, G, H, I, J, K, L, and N. Genotypes E and M were previously recognized but were abolished due to phylogenetic analysis that MuVs assigned to them instead belonged genotypes C and K, respectively.

The different genotypes vary in frequency from region to region. For example, genotypes C, D, H, and J are more common in the western hemisphere, whereas genotypes F, G, and I are more common in Asia, although genotype G is considered to be a global genotype. Genotypes A and B have not been observed in the wild since the 1990s. This diversity of MuV is not reflected in the antibody response since because there is only one serotype, antibodies to one genotype are also functional against all others.

The disclosed compounds, compositions, and methods can be used to treat any one or more of mumps virus genotypes A, B, C, D, F, G, H, I, J, K, L, and N.

c. Measles Virus

Measles morbillivirus (MeV), also called measles virus (MV), is a single-stranded, negative-sense, enveloped, non-segmented RNA virus of the genus Morbillivirus within the family Paramyxoviridae. It is the cause of measles. The measles virus genome is typically 15,894 nucleotides long and encodes eight proteins. The WHO currently recognizes 8 clades of measles (A-H). Subtypes are designed with numerals-A1, D2 etc. 24 subtypes are recognized (Bianchi, et al., Epidemiol Infect., 147: e80 (2019)). Despite the variety of measles genotypes, there is only one measles serotype. The following 19 genotypes have been detected since 1990: A*, B2, B3, C1, C2, D2, D3, D4, D5, D6, D7, D8, D9, D10, D11, G2, G3, H1, H2 (Centers for Disease Control Website, “Measles (Rubeola).” Four predominant measles genotypes currently circulating worldwide: D8, B3, H1 and D4. Antibodies to measles bind to the hemagglutinin protein. Thus, antibodies against one genotype (such as the vaccine strain) protect against all other genotypes.

The disclosed compounds, compositions, and methods can be used to treat any one or more of measles viruses of any of the eight clades and/or 24 subtypes.

d. Zika Virus

Zika virus is a mosquito-borne virus belonging to the family Flaviviridae and the genus Flavivirus, thus is related to the dengue, yellow fever, Japanese encephalitis, and West Nile viruses. Like other flaviviruses, Zika virus is enveloped and icosahedral and has a nonsegmented, single-stranded, 10 kilobase, positive-sense RNA genome. It is most closely related to the Spondweni virus and is one of the two known viruses in the Spondweni virus clade. There are two Zika lineages: the African lineage and the Asian lineage. Phylogenetic studies indicate that the virus spreading in the Americas is 89% identical to African genotypes, but is most closely related to the Asian strain that circulated in French Polynesia during the 2013-2014 outbreak. The disclosed compounds, compositions, and methods can be used to treat any one or more Zika viruses, including those of the African and/or Asian lineages.

e. Dengue Virus

Dengue virus is the cause of dengue fever. It is a mosquito-borne, single positive-stranded RNA virus of the family Flaviviridae; genus Flavivirus. Four serotypes of the virus have been found, a reported fifth has yet to be confirmed, all of which can cause the full spectrum of disease. The disclosed compounds, compositions, and methods can be used to treat any one or more dengue viruses, including and serotype thereof.

f. Influenza Virus

In some embodiments, the virus is a member of the family Orthomyxoviridae, for instance, a member of the genus Influenzavirus A, a member of the genus Influenzavirus B, a member of the genus Influenzavirus C, or a member of the genus Thogotovirus. Type species that are members of the genus Influenzavirus A, include, but are not limited to, Influenza A virus.

There are 18 different hemagglutinin subtypes and 11 different neuraminidase subtypes (H1 through H18 and N1 through N11, respectively). While there are potentially 198 different influenza A subtype combinations, only 131 subtypes have been detected in nature. Exemplary serotypes of the type species Influenza virus A include, but are not limited to, H1N1, H1N2, H2N2, H3N1, H3N2, H3N8, H5N1, H5N2, H5N3, H5N8, H5N9, H7N1, H7N2, H7N3, H7N4, H7N7, H9N2, and H10N7. The skilled person will recognize that other serotypes are possible in view of antigenic drift and the simultaneous infection of one animal with different influenza viruses.

Current subtypes of influenza A viruses that routinely circulate in people include: A(H1N1) and A(H3N2). Currently circulating influenza A(H1N1) viruses are related to the pandemic 2009 H1N1 virus that emerged in the spring of 2009 and caused a flu pandemic (CDC 2009 H1N1 Flu website). This virus, scientifically called the “A(H1N1)pdm09 virus,” and more generally called “2009 H1N1,” has continued to circulate seasonally since then. These H1N1 viruses have undergone relatively small genetic changes and changes to their antigenic properties (i.e., the properties of the virus that affect immunity) over time.

Type species that are members of the genus Influenzavirus B include, but are not limited to, Influenza B virus. Type species that are members of the genus Influenzavirus C include, but are not limited to, Influenza C virus. Type species that are members of the genus Thogotovirus include, but are not limited to, Thogoto virus and Dori virus. Serotypes of the type species Dhori virus include, but are not limited to, Batken virus and Dhori virus.

In some embodiments, the subject has an influenza infection. See, e.g., Perwitasari, et al., Antimicrob Agents Chemother, 57(1):475-83 (2013). doi: 10.1128/AAC.01532-12.)). For example, in some embodiments, the subject has an influenza (e.g., influenza A, influenza B, influenza C, and/or influenza D) infection and an infection from another virus, such as a coronavirus. In some embodiments, the subject does not have an influenza viral infection.

g. Coronavirus

In some embodiments, the virus is a coronavirus. The current classification of coronaviruses recognizes 39 species in 27 subgenera, five genera and two subfamilies that belong to the family Coronaviridae, suborder Cornidovirineae, order Nidovirales and realm Riboviria (Coronaviridae Study Group of the International Committee on Taxonomy of Viruses, Nat Microbiol 2020. DOI: 10.1038/s41564-020-0695-z). They are enveloped viruses with a positive-sense single-stranded RNA genome and a nucleocapsid of helical symmetry. The genome size of coronaviruses ranges from approximately 26 to 32 kilobases, one of the largest among RNA viruses.

Coronaviruses cause diseases in mammals and birds. Most typically, alphacoronaviruses and betacoronaviruses infect mammals, while gammacoronaviruses and deltacoronaviruses primarily infect birds. At least seven of these viruses can infect people: 229E (alpha) NL63 (alpha) OC43 (beta) HKU1 (beta) MERS-CoV (beta) virus, SARS-CoV (beta), and SARS-CoV-2 (beta).

Coronavirus species and representative viruses thereof include [representative virus (of species)]: SARSr-CoV BtKY72 (Severe acute respiratory syndrome-related coronavirus), SARS-CoV-2 (Severe acute respiratory syndrome-related coronavirus), SARSr-CoV RaTG13 (Severe acute respiratory syndrome-related coronavirus), SARS-CoV PC4-227 (Severe acute respiratory syndrome-related coronavirus), SARS-CoV (Severe acute respiratory syndrome-related coronavirus), Bat-Hp-BetaCovC (Bat Hp-betacoronavirus Zhejiang2013), Ro-BatCoV GCCDCl (Rousettus bat coronavirus GCCDCl), Ro-BatCoV HKU9 (Rousettus bat coronavirus HKU9), Ei-BatCoV C704 (Eidolon bat coronavirus C704), Pi-BatCoV HKU5 (Pipistrellus bat coronavirus HKU5), Ty-BatCoV HKU4 (Tylonycteris bar coronavirus HKU4), MERS-CoV (Middle East respiratory syndrome-related coronavirus), EriCoV (Hedgehog coronavirus), MHV (murine coronavirus), HCoV HKU1 (Human coronavirus HKU1), ChRCoV HKU24 (China Rattus coronavirus HKU24), ChRCovC HKU24 (Betacoronavirus 1), MrufCoV 2JL14 (Myodes coronavirus 2JL14), HCoV NL63 (Human coronavirus NL63), HCoV 229E (Human coronavirus 229E), and HCoV C43 (Human coronavirus OC43). See, e.g., Coronaviridae Study Group of the International Committee on Taxonomy of Viruses, Nat Microbiol 2020. DOI: 10.1038/s41564-020-0695-z), which is specifically incorporated by reference in its entirety. In some embodiments, the coronavirus is a common cold coronavirus such as 229E, NL63, OC43, and HKU1.

In particularly preferred embodiments, the virus is a Severe acute respiratory syndrome-related virus, such as, SARSr-CoV BtKY72, SARS-CoV-2, SARSr-CoV RaTG13, SARS-CoV PC4-227, or SARS-CoV, preferably one that infects humans such as SARS-CoV or SARS-CoV-2.

In some embodiments, the virus is a Middle East respiratory syndrome-related virus such as MERS-CoV.

In some embodiments the virus is a SARS-CoV-2. The sequence WIV04/2019, belonging to the GISAID S clade/PANGO A lineage/Nextstrain 19B clade, is thought to most closely reflect the sequence of the original SARS-CoV-2 infecting humans. It is known as “sequence zero”, and is widely used as a reference sequence. Subsequent to the initial isolate from Wuhan, China, numerous SARS-CoV-2 virus sequence variants of WIV04/2019 have been identified, some of which may be of particular importance due to their potential for increased transmissibility, increased virulence, and reduced effectiveness of vaccines against them. SARS-CoV-2 having a variation of the WIV04/2019 sequence include, but are not limited to:
B.1.1.7 lineage (a.k.a. 20I/501Y.V1 Variant of Concern (VOC) 202012/01). This variant has a mutation in the receptor binding domain (RBD) of the spike protein at position 501, where the amino acid asparagine (N) has been replaced with tyrosine (Y). The shorthand for this mutation is N501Y. This variant also has several other mutations, including: 69/70 deletion: occurred spontaneously many times and likely leads to a conformational change in the spike protein.
P681H: near the S1/S2 furin cleavage site, a site with high variability in coronaviruses. This mutation has also emerged spontaneously multiple times.
B.1.351 lineage (a.k.a. 20H/501Y.V2). This variant has multiple mutations in the spike protein, including K417N, E484K, N501Y. Unlike the B.1.1.7 lineage detected in the UK, this variant does not contain the deletion at 69/70.
P.1 lineage (a.k.a. 20J/501Y.V3). The P.1 variant is a branch off the B.1.1.28 lineage that was first reported by the National Institute of Infectious Diseases (NIID) in Japan in four travelers from Brazil, sampled during routine screening at Haneda airport outside Tokyo. The P.1 lineage contains three mutations in the spike protein receptor binding domain: K417T, E484K, and N501Y.
Omicron lineage. B.1.1.529, BA.1, BA.1.1, BA.2, BA.3, BA.4 and BA.5 lineages. Spike Protein Substitutions include A67V, del69-70, T95I, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. Omicron variants are described having potential increased transmissibility, potential reduction in neutralization by some EUA monoclonal antibody treatments, a potential reduction in neutralization by post-vaccination sera. CDC website, “SARS-CoV-2 Variant Classifications and Definitions,” updated Apr. 26, 2022.

Other lineages and mutations of interest include, but are not limited to, B.1.1.207, B.1.429, B.1.427, B.1.525, and other two-mutation (e.g., N501T-G142D), or three-mutation (e.g., N501T-G142D-F486L) variants in the Spike protein. All of these lineages and sequence alternatives relative to the WIV04/2019 strain are also considered SARS-CoV-2 virus. In some embodiments, SARS-CoV-2 is a strain or isolate with elevated or potentially elevated risk for causing human disease relative to WIV04/2019. See, e.g., Science Brief, Emerging SARS-CoV-2 variants (CDC website, Updated Jan. 28, 2021), Horby, et al., “NERVTAG note on B.1.1.7 severity.” SAGE meeting report. Jan. 21, 2021; Wu, et al. “mRNA-1273 vaccine induces neutralizing antibodies against spike mutants from global SARS-CoV-2 variants.” bioRxiv. Posted Jan. 25, 2021; Xie, et al., “Neutralization of N501Y mutant SARS-CoV-2 by BNT162b2 vaccine-elicited sera.” bioRxiv. Posted Jan. 7, 2021; Greaney, et al. “Comprehensive mapping of mutations to the SARS-CoV-2 receptor-binding domain that affect recognition by polyclonal human serum antibodies.” bioRxiv. [Preprint posted online Jan. 4, 2021]; Weisblum, et al., “Escape from neutralizing antibodies by SARS-CoV-2 spike protein variants.” eLife 2020; 9:e61312; Resende, at al. “Spike E484K mutation in the first SARS-CoV-2 reinfection case confirmed in Brazil,” 2020. [Posted on virological.org on Jan. 10, 2021]

Various strains and isolates of the foregoing viruses are known and include the representative genomic sequences provided as, for example, GenBank Accession Nos. MN908947.3 (SEQ ID NO:1), MN985325.1 (SEQ ID NO:2), AY274119.3 (SEQ ID NO:3), or JX869059.2 (SEQ ID NO:4), other accession numbers provided herein, and those sequences and accession numbers provided in, e.g., Coronaviridae Study Group of the International Committee on Taxonomy of Viruses, Nat Microbiol 2020. DOI: 10.1038/s41564-020-0695-z), as well as NCBI and GISAID which provide hundreds of SARS-CoV-2 sequences, all of which are specifically incorporated by reference herein in their entireties.

In some embodiments, the SARS-CoV-2 has 1, 2, 3, 4, 5, 6, 7, 8, 9, 10, 11, 12, 13, 14, 15, 16, 17, 18, 19, 20, 21, 22, 23, 24, 25, or more mutations in its spike protein relative to e.g., WIV04/2019, SEQ ID NO:1, and/or SEQ ID NO:2 or another reference sequence such as those provided therein. The spike protein sequence for SEQ ID NO:1 is

(SEQ ID NO: 5)
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNFRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPR
RARSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIANQFNSA
IGKIQDSLSSTASALGKLQDVVNQNAQALNTLVKQLSSNFGAISSVLNDILSRLDKVEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAIVMVTIMLCCMTSC
CSCLKGCCSCGSCCKFDEDDSEPVLKGVKLHYT

which is encoded by 21563..25384 of GenBank: MN908947.3 (SEQ ID NO:1), /gene=“S”, /note=“structural protein”, /codon_start=1/product=“surface glycoprotein”, /protein_id=“QHD43416.1”).

In some embodiments, the spike protein of the SARS-CoV-2 has at least 70%, 75%, or 80%, preferably at least 85%, more preferably at least 86%, 87%, 88%, 89%, 90%, 91%, 92%, 93%, 94%, 95%, 96%, 97%, 98%, 99%, or 100% sequence identity to the spike protein of one or more of SEQ ID NOS:1 or 2, or another viral accession number provided herein.

In some embodiments, the spike protein of the SARS-CoV-2 has at least 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO:5.

Mutations can be substitutions, insertions, deletions, or a combination thereof. Exemplary mutations include those discussed herein, e.g., one or more of the following mutations in the spike protein sequence:
H69, optionally deletion thereof;
V70, optionally deletion thereof;
G142, optionally G142D;
K417, optionally K417N or K417T;
E484, optionally E484K;
F486, optionally F486L; and/or
N501 mutation, optionally N501Y or N501T.
   These mutations are provided individually and in all combinations. These residues are illustrated with bold/italics/shading in SEQ ID NO:5 above.

In some embodiments, the SARS-CoV-2 is of the B.1.1.7, B.1.351, P.1, B.1.1.207, B.1.429, B.1.427, or B.1.525 lineage.

In an exemplary embodiment, the SARS-CoV-2 is isolate USA/CA_CDC_5574/2020, or another isolate sharing one or mutations therewith relative to the original Wuhan isolate. Under the nomenclature system introduced by GISAID (Global Initiative on Sharing All Influenza Data), SARS-CoV-2, isolate USA/CA_CDC_5574/2020 is assigned lineage B.1.1.7 and GISAID clade GR using Phylogenetic Assignment of Named Global Outbreak LINeages (PANGOLIN) tool (GISAID website, 3. Rambaut, et al., Nat. Microbiol. 5 (2020): 1403-1407. PubMed: 32669681; Mercatelli, et al., Front. Microbiol. (2020): doi.org/10.3389/fmicb.2020.01800. PubMed: 32793182. The complete genome of SARS-CoV-2, isolate USA/CA_CDC_5574/2020 has been sequenced (GISAID: EPI_ISL_751801). The following mutations are present in the clinical isolate:

Spike A570D, Spike D614G, Spike D1118H, Spike H69del, Spike N501Y, Spike P681H, Spike S982A, Spike T716I, Spike V70del, Spike Y145del, M (Membrane protein) V70L, N (Nucleocapsid protein) D3L, N G204R, N R203K, N S235F, NS3 T223I, NS8 (Non-structural protein 8) Q27stop, NS8 R52I, NS8 Y73C, NSP3 (Non-structural protein 3) A890D, NSP3 I1412T, NSP3 T183I, NSP6 (Non-structural protein 6) F108del, NSP6 G107del, NSP6 S106del, NSP12 (Non-structural protein 12) P323L, NSP13 (Non-structural protein 13) A454V, NSP13 K460R. One additional SNP in ORFIab L3826F was reported in the deposited passage two virus, in comparison to the clinical specimen. See also BEI Resources, Catalog No. NR-54011, and its description, which is specifically incorporated by reference herein in its entirety.

These, however, are non-limiting examples, and the disclosed compositions and methods can also be used to treat other strains of coronavirus, particularly SARS and MERS coronaviruses. In some embodiments, the (DNA sequence) of the viral genome has a sequence at least 70%, 75%, or 80%, preferably at least 85%, more preferably at least 86%, 87%, 88%, 89%, 90%, 91%, 92%, 93%, 94%, 95%, 96%, 97%, 98%, 99%, or 100% sequence identity to one or more of SEQ ID NOS:1, 2, 3, or 4, or another viral accession number provided herein, or a sequence or accession number provided in Coronaviridae Study Group of the International Committee on Taxonomy of Viruses, Nat Microbiol 2020. DOI: 10.1038/s41564-020-0695-z, all of which are specifically incorporated by reference herein in their entireties. It will be appreciated that the sequences are provided as DNA sequences, but the viral genome itself will typically have the corresponding RNA sequences. Thus, the corresponding RNA sequences are also expressly provided herein.

GenBank Accession No. MN908947.3, NCBI Accession No. NC_045512.2, which is specifically incorporated by reference herein in its entirety, provides the (DNA) genomic sequence for SARS-CoV-2 (Severe acute respiratory syndrome coronavirus 2 isolate Wuhan-Hu-1, complete genome): (SEQ ID NO:1).

GenBank Accession No. MN985325.1, which is specifically incorporated by reference herein in its entirety, provides the (DNA) genomic sequence for SARS-CoV-2 (Severe acute respiratory syndrome coronavirus 2 isolate 2019-nCoV/USA-WA1/2020, complete genome): (SEQ ID NO:2).

GenBank Accession No. GenBank: AY274119.3, which is specifically incorporated by reference herein in its entirety, provides the (DNA) genomic sequence for SARS-CoV (Severe acute respiratory syndrome-related coronavirus isolate Tor2, complete genome): (SEQ ID NO:3).

GenBank Accession No. GenBank: JX869059.2, which is specifically incorporated by reference herein in its entirety, provides the (DNA) genomic sequence for MERS-CoV (Human betacoronavirus 2c EMC/2012, complete genome): (SEQ ID NO:4).

In some embodiments, the subject is diagnosed with a positive SARS-CoV-2 viral test result and has at least one mild or moderate COVID-19 symptom (i.e. fever or chills, cough, shortness of breath or difficulty breathing, fatigue, muscle or body aches, headache, new loss of taste or smell, sore throat, congestion or runny nose, nausea, vomiting or diarrhea) for 6 days or less prior to the first administration of the probenecid, metabolite or analog or prodrug thereof or pharmaceutically acceptable salt thereof.

In some embodiments, the subject does not have a coronavirus viral infection.

### B. Exemplary Subjects

Subjects can be male and/or female. Subject can be adults (e.g., 18 or over), and/or children under 18 years old.

The age of the subject can range from the young children, including pediatric subjects, to the elderly. Thus, the formulations can be administered to an adult patient, or a pediatric patient. In embodiments, the pediatric patient can be 0 to 18 years of age or any integer or fractional subrange or specific number therebetween. For example, in some embodiments the subject is 2 to 10 years of age, or 8 to 17 years of age, or 12 to 17 years of age, or 8 to 11 years of age, or 1 month to 7 years of age, or 0.5 years to 8 years of age, or 6 years of age or younger, or 4 years of age or younger, or 2 years of age or younger. In some embodiments, the children are under two years of age. In some embodiments, the subject is at least 2 years old, and optionally has one or more symptoms, e.g., cough fever, and/or other discussed herein, etc. Treatment may be particularly indicated if the subject is male and/or over the age of 40, 50, 60, 70 or 80. In some embodiments, the subject is obese (BMI of over 30, where BMI is Body Mass Index, calculated as a person's weight in kilograms divided by the square of his or her height in meters).

In some embodiments, the subject has an underlying condition such as asthma, heart disease, diabetes, cancer, chronic lung disease, chronic heart disease, chronic kidney disease, vitamin A deficiency, immune-compromised, or a combination thereof.

In some embodiments, the subject is a human. In some embodiments, a group of humans is treated together. Certain viruses including, but not limited to, flu, SARS-CoV-2, and measles can be highly contagious. For example, measles is highly contagious and spreads through the air when an infected person coughs or sneezes. It is so contagious that if one person has it, 9 out of 10 people of all ages around him or her will also become infected if they are not protected (CDC.gov “Measles is Highly Contagious Infographic”). Although the vaccine is highly effective at controlling measles virus, in recent years there has been a resurgence of measles in the US (Dimala, et al., Scientific Reports volume 11, Article number: 51 (2021)). This has resulted in growing concerns of a potential re-establishment of transmission of measles, and loss of the ‘measles elimination’ status by the US in the years to come. There are indications this resurgence could be due to the declining vaccine coverage as a result of vaccine hesitancy. Additionally, given the highly infectious nature of measles, several factors could potentially favor the transmission of measles and consequent resurgence such as; population density; inter/intra-age contact; timing of the vaccination and waxing conferred immunity. Because measles is highly contagious beginning about four days before the rash appears, subjects with the virus can spread it to other before knowing they are infected. This problem is particularly salient in institutional settings such as schools, hospitals, prisons, churches, and other locations where large numbers of people gather. Thus, in some embodiments, the disclosed methods include treating a group of people. The group of people can be infected by virus, uninfected, or a combination thereof. The treatment thus may be prophylactic to some subjects and/or therapeutic to some subjects in the group. Because the disclosed compounds can be used for prophylactic and therapeutic treatment of viruses, all subjects of groups can be advantageously treated together regardless of their status. In some embodiments, the group is or was present together in an institution such a school, hospital, prison, church, and/or a group unable to, or reluctant to, engage in social distancing such as children, e.g., 2-10 years, 2-18 years, etc. as discussed above, military units, etc. Thus, for non-limiting example, in some embodiments, a class of students, optionally pediatric aged students, is treated after being exposed to a classmate that has been diagnosed as a having a viral infection. Exemplary viruses include all of those disclosed herein. In some embodiments the virus is a highly infectious virus and/or a highly virulent virus. In particular embodiments, the virus is measles or SARS-CoV-2.

In other embodiments, the subject is a non-human mammal or a bird. For example, the non-human mammal can be a member of the family Muridae (a murine animal such as rat or mouse), a primate, (e.g., monkey, human), a gerbil, a guinea pig, a ferret, or a swine species. The subject can be an avian species, including but not limited to, gulls, terns, and shorebirds, waterfowl, such as swans, ducks, and geese, chickens, turkeys, wild backyard birds, and pigeons. The subject can be a dog or cat, an agricultural animal such as cattle, poultry (e.g., chickens), sheep, pigs, goats, horses, etc.

For example, birds, just like people, get the flu. Bird flu viruses infect birds, including chickens, other poultry, and wild birds such as ducks. Usually bird flu viruses only infect other birds. It is rare for people to get infected with bird flu viruses, but it can happen. Two types, H5N1 and H7N9, have infected some people during outbreaks in Asia, Africa, the Pacific, the Middle East, and parts of Europe. Thus disclosed herein are methods of administering an effective amount of probenecid, a metabolite, analog, or prodrug thereof, or a pharmaceutically acceptable salt thereof to treat flu, including but not limited to H5N1 and H7N9, in birds including but not limited to agricultural poultry such as chickens and turkeys.

Poultry are domesticated birds that are raised by farmers for meat and eggs. Poultry includes, without limitation, chickens, ducks, geese, turkeys, guinea fowl, and pheasants. An example of a commercially raised duck is the White Pekin duck.

Examples of commercially raised geese are Embden, Toulouse, Chinese goose, African goose, Sebastopol, Pilgrim, and American Buff breeds. Examples of commercially raised turkeys include White, Hollands, Bronze, Narragansett, Bourbon Red, Black, Slate, Royal Palm, Beltsville, and Small White breeds.

Examples of commercially raised chickens include the American Class, the Asiatic Class, the English Class, and the Mediterranean Class. The American Class includes Buckeye, Chantecler, Delaware, Doninique, Holland, Java, Jersey Giant, Lamona, New Hampshire, Plymouth Rock, Rhode Island Red, Rhode Island White, and Wyandotte breeds. The Asiatic Class includes the Brahma, Cochin, and Langshan breeds. The English Class includes the Australorp, Cornish, Dorking, Orpington, Redcap, and Sussex breeds. The Mediterranean Class includes the Ancona, Blue Andalusian, Catalanas, Leghorn, Minorca, Spanish, and Buttercup breeds. As would be appreciated by those of skill in the art, there are classes and breeds of poultry other than those listed above. The disclosed compositions can be used on all classes and breeds of poultry.

Similarly, swine influenza is a respiratory disease of pigs caused by type A influenza viruses that regularly cause outbreaks of influenza in pigs. Influenza viruses that commonly circulate in swine are called “swine influenza viruses” or “swine flu viruses.” Like human influenza viruses, there are different subtypes and strains of swine influenza viruses. The main swine influenza viruses circulating in U.S. pigs in recent years have been, swine triple reassortant (tr) H1N1 influenza virus, trH3N2 virus, and trH1N2 virus. Thus disclosed herein are methods of administering an effective amount of probenecid, a metabolite, analog, or prodrug thereof, or a pharmaceutically acceptable salt thereof to treat flu, including but not limited to H5N1 and H7N9, in swine (i.e., pigs).

A preferred method of administration to non-human mammals and birds is by mouth, e.g., in the animal's water or feed.

The methods include both preventing and/or therapeutically treating viral infections in non-human animals, particularly non-human mammals and birds. The methods can be practiced on a single animal, or on multiple animals, which may optionally be reared together and, further optionally wherein all animals reared together may be aged matched to within a month, a week, or less, such as within 6, 5, 4, 3, 2 or 1 days of each other.

For example, the methods can be practiced on a group of up to, about, or at least, 2, 3, 4, 5, 6, 7, 8, 9, 10, 15, 20, 25, 30, 35, 40, 45, 50, 55, 60, 70, 80, 90, 100, 150, 200, 250, 300, 400, 500, 600, 700, 800, 900, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶ or more, and all animals in the group can be optionally age matched as indicated above. The term “about” in this context can mean within ±50%, ±40%, ±30%, ±20%, ±10%, ±5%, ±4%, ±3%, ±2%, ±1% or less of the stated value.

The treated animals can be healthy animals, for example, animals which are not infected with virus. In another embodiment, the animals are unhealthy animals, for example, animals which are infected with virus. In some embodiments, the animals are or include animals exposed to unhealthy (e.g., virally infected) animals. In some embodiments a mixture of healthy and unhealthy animals are treated together in a group.

Additionally or alternatively, probenecid may be given to improve weight gain in animals (important in swine and chickens) as probenecid may improve antibiotic efficacy in animals (e.g., chicken and pigs) and may lower inflammation in addition to treating or preventing infection of influenzas and other viruses.

### C. Exemplary Symptoms and Infections

In some embodiments, the subject presents and/or the disclosed compounds, compositions, and methods are effective to treat, one or more symptoms associated with one or more viruses for which treatment is desired.

For example, RSV infection can present with a wide variety of signs and symptoms that range from mild upper respiratory tract infections (URTI) to severe and potentially life-threatening lower respiratory tract infections (LRTI) requiring hospitalization and mechanical ventilation. Most childhood RSV infections may include one or more of nasal congestion, runny nose, cough, and low-grade fever. Inflammation of the nasal mucosa (rhinitis) and throat (pharyngitis), as well as redness of the eyes (conjunctival infection), may be seen, and bronchiolitis can occur. Reinfection in adulthood often produces only mild to moderate symptoms similar to the common cold or sinus infection. Infection may also be asymptomatic. If present, symptoms are generally isolated to the upper respiratory tract: runny nose, sore throat, fever, and malaise.

Mumps virus infection leads to fever, muscle pain, and painful swelling of the parotid glands, two salivary glands situated on the sides of the mouth in front of the ears. Infection may also involve many other tissues and organs, resulting in a variety of inflammatory reactions such as encephalitis, aseptic meningitis, orchitis, myocarditis, pancreatitis, nephritis, oophoritis, and mastitis. Mumps is usually not life-threatening and typically resolves within a few weeks after the onset of symptoms, but long-term complications such as paralysis, seizures, hydrocephalus, and deafness can occur.

Measles is a highly contagious infectious disease caused by measles virus. Symptoms usually develop 10-12 days after exposure to an infected person and last 7-10 days. Initial symptoms typically include fever, often greater than 40° C. (104° F.), cough, runny nose, and inflamed eyes. Small white spots known as Koplik's spots may form inside the mouth two or three days after the start of symptoms, followed by a red, flat rash which usually starts on the face and then spreads to the rest of the body typically beginning three to five days after the start of symptoms. Common complications include diarrhea, middle ear infection, and pneumonia, which occur in part due to measles-induced immunosuppression, and less commonly seizures, blindness, or inflammation of the brain may occur.

Zika fever (also known as Zika virus disease) is an illness caused by Zika virus. Most cases have no symptoms, but when present they are usually mild and can resemble dengue fever. Symptoms may include fever, red eyes, joint pain, headache, and a maculopapular rash. Symptoms generally last less than seven days, and no deaths have been reported related to initial infection.

Infection during pregnancy causes microcephaly and other brain malformations in some babies, an infection in adults has been linked to Guillain-Barré syndrome (GBS) and Zika virus has been shown to infect human Schwann cells.

Dengue virus causes dengue fever disease, as referred to as breakbone fever, vomiting and dandy fever; and dengue hemorrhagic fever and dengue shock syndrome referring to severe forms. Signs and symptoms may include severe headache; retro-orbital pain; muscle, joint, and bone pain; macular or maculopapular rash; and minor hemorrhagic manifestations, including petechiae, ecchymosis, purpura, epistaxis, bleeding gums, hematuria, or a positive tourniquet test result. Allergic symptoms are one of the core symptoms that are highly associated with dengue severity.

Influenza, commonly known as “the flu”, is an infectious disease caused by influenza viruses. Symptoms range from mild to severe and often include fever, runny nose, sore throat, muscle pain, headache, coughing, and fatigue. These symptoms begin from one to four days after exposure to the virus (typically two days) and last for about 2-8 days. Diarrhea and vomiting can occur, particularly in children. Influenza may progress to pneumonia, which can be caused by the virus or by a subsequent bacterial infection. Other complications of infection include acute respiratory distress syndrome, meningitis, encephalitis, and worsening of pre-existing health problems such as asthma and cardiovascular disease.

In humans, coronaviruses can cause respiratory tract infections that can range from mild to lethal. Mild illnesses include some cases of the common cold, while more lethal varieties can cause SARS, MERS, and COVID-19 (i.e., caused by SARS-CoV-2).

The subject may have one or more symptoms characteristic of SARS, MERS, or COVID-19.

SARS (i.e., SARS-CoV) usually begins with flu-like signs and symptoms such as fever, chills, muscle aches, headache and occasionally diarrhea. After about a week, signs and symptoms include fever of 100.5 F (38 C) or higher, dry cough, and shortness of breath.

Reported illnesses from COVID-19 (i.e., caused by SARS-CoV-2) have ranged from mild symptoms to severe illness and death for confirmed cases. The most common symptoms are fever, tiredness, dry cough, anosmia (loss of taste and/or smell) and shortness of breath. Runny nose, vomiting, diarrhea, skin rash (particularly on toes and fingers), sore throat, fatigue, muscle or body aches, headache, and sore throat have also been reported. These symptoms may appear 2-14 days after exposure.

Most people confirmed to have MERS-CoV infection have had severe respiratory illness with symptoms of fever, cough, and/or shortness of breath. Some people also had diarrhea and nausea/vomiting. For many people with MERS, more severe complications followed, such as pneumonia and kidney failure. Some infected people had mild symptoms (such as cold-like symptoms) or no symptoms at all.

In some embodiments, the subject has at least one mild or moderate COVID-19 symptom such as fever or chills, cough, shortness of breath or difficulty breathing, fatigue, muscle or body aches, headache, new loss of taste or smell, sore throat, congestion or runny nose, nausea, vomiting or diarrhea for 6, 5, 4, 3, 2, or 1 day(s) or less prior has treatment.

Symptoms caused by coronavirus infection in non-human species vary: in chickens, they cause an upper respiratory tract disease, while in cows and pigs they cause diarrhea.

In some embodiments, the subject has post-COVID condition (PCC) or long-COVID (also referred to as long-haul COVID, post-acute COVID-19, post-acute sequelae of SARS CoV-2 infection (PASC), long-term effects of COVID, and chronic COVID). Post-COVID conditions are a wide range of new, returning, or ongoing health problems that people experience after first being infected with SARS-CoV-2. Most people with COVID-19 get better within a few days to a few weeks after infection, so at least four weeks after infection is the start of when post-COVID conditions could first be identified. Anyone who was infected can experience post-COVID conditions. Most people with post-COVID conditions experienced symptoms days after their SARS CoV-2 infection when they knew they had COVID-19, but some people with post-COVID conditions did not notice when they first had an infection.

### D. Other Indications

The disclosed compounds, compositions, and methods can also be used to treat a variety of other diseases, disorders, and indications. For examples, in some embodiments, a formulation of probenecid, or probenecid metabolite, analog, prodrug, or pharmaceutically acceptable salt thereof, or formulation thereof, is used to treat a subject with a condition previously identified to be treatable with probenecid.

For example, probenecid is a medication used to treat gouty arthritis, tophaceous gout, and hyperuricemia. It inhibits the renal excretion of organic anions and reduces tubular reabsorption of urate. Probenecid has also been used to treat patients with renal impairment, and, because it reduces the renal tubular excretion of other drugs, has been used as an adjunct to antibacterial therapy. It is a uricosuric and renal tubular blocking agent and is used in combination with colchicine to treat chronic gouty arthritis when complicated by frequent, recurrent acute attacks of gout. It inhibits the reabsorption of urate at the proximal convoluted tubule, thus increasing the urinary excretion of uric acid and decreasing serum urate levels. Effective uricosuria reduces the miscible urate pool, retards urate deposition, and promotes resorption of urate deposits. At the proximal and distal tubules, probenecid competitively inhibits the secretion of many weak organic acids including penicillins, most cephalosporins, and some other β-lactam antibiotics. This results in an increase in the plasma concentrations of acidic drugs eliminated principally by renal secretion, but only a slight increase if the drug is eliminated mainly by filtration. Thus, the drug can be used for therapeutic advantages to increase concentrations of certain β-lactam antibiotics in the treatment of gonorrhea, neurosyphilis, or pelvic inflammatory disease (PID). For the treatment of uncomplicated gonorrhea in men or women, a single 1000 mg dose of Probenecid (2 tablets) may be given with adequate doses of oral ampicillin, intramuscularly injected aqueous procaine penicillin G or cefoxitin. If oral ampicillin is used, probenecid can be administered simultaneously. If a parenteral antibiotic is administered, the dose of probenecid can be given preferably at least 30 minutes before the injection. In some embodiments, the subject has a bacterial infection. Thus, in some embodiments, a subject is administered an effective amount of a metabolite, analog, or prodrug of probenecid in addition to, or in place of, probenecid to treat on or more of the foregoing diseases, disorders, or conditions.

### E. Exemplary Dosages and Regimens

Probenecid, metabolites, analogs and prodrugs thereof and pharmaceutically acceptable salts thereof can be administered to a subject in a pharmaceutical composition, such as those discussed above, and can be administered by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes, as discussed in more detail above. Contemplated routes and strategies of administration include, but are not limited to, oral, buccal, nasal, transdermal, injectable, slow release, controlled release, iontophoresis, sonophoresis, and other delivery devices and methods. Injectable methods include, but are not limited to, parenteral routes of administration, intravenous, intramuscular, subcutaneous, intraperitoneal, intraspinal, intrathecal, intracerebroventricular, intraarterial and other routes of injection.

The compounds can also be delivered transdermally as a continuous release drug delivery system through microneedles. In the microneedle system for transdermal delivery of the compound, a transdermal patch with microneedles facilitates delivery of a drug through skin of a subject, said apparatus capable of generating at least one micro-channel in an area on the skin of the subject, and the patch comprising a pharmaceutical composition comprising probenecid and a cyclodextrin molecule that enhances the solubility of probenecid in aqueous solution.

The precise dosage will vary according to a variety of factors such as subject-dependent variables (e.g., age, immune system health, clinical symptoms, route of delivery, etc.). Controlled, slow release, or sustained release of the therapeutic compound over a predetermined period of time are also contemplated. Administration of the composition using these formulations allows for a desired concentration of the drug to be maintained in the bloodstream of the patient for a longer period of time than with conventional formulations. Slow-release, controlled or sustained release formulations are known to those skilled in the art and include formulations such as coated tablets, pellets, capsules, dispersion of the active agent in a medium that is insoluble in physiologic fluids or where the release of the active agent is released after degradation of the formulation due to mechanical, chemical or enzymatic activity.

For pediatric doses, syrups are generally preferred and visually appealing to increase the patient compliance. However, probenecid is water insoluble and formulation in syrups are less stable. Therefore, a method for manufacturing a sustained release syrup formulation is provided. The syrup formulation includes a water insoluble polymer and probenecid in water, wherein the water insoluble polymer can be ethyl cellulose, polyvinylacetate, hydroxy methyl cellulose etc. After dissolving in an organic solvent, the solution containing the water insoluble polymer and probenecid are spray-dried to form microparticles which are dispersed in a sugar syrup to form a dispersion in a homogenously mixed state.

For treating gout, probenecid has been administered at 250 mg Per os/oral (PO) twice daily for 1 week; increasing to 500 mg PO twice daily to 2 g/day maximum with dosage increases of 500 mg.

For prolonging penicillin serum levels, probenecid has been administered at 500 mg PO four times daily.

For pelvic inflammatory disease probenecid has been administered at 1 g PO with 2 g cefoxitin intramuscular (IM) as single dose.

For gonorrhea, probenecid has been administered at 1 g PO with 2 g cefoxitin IM as single dose.

A typically pediatric (e.g., age: 2 to 14 years and weight less than 50 kg) administration as an adjuvant to antibiotic therapy is Initial: 25 mg/kg (or 0.7 g/m2) orally once; Maintenance: 40 mg/kg (or 1.2 g/m2) per day orally administered in 4 equally divided doses 4 times a day.

Thus, in general, by way of example only, dosage forms useful in the disclosed methods may include doses in the range of 0.1 mg to 3,000 mg; 25 mg to 2,000 mg; 25 mg to 1,000 mg; 50 mg to 1,000 mg; 100 mg to 1,000 mg; or 250 mg to 1,000 mg, with doses of 10 mg, 25 mg, 45 mg, 50 mg, 100 mg, 150 mg, 200 mg, 250 mg, 300 mg, 350 mg, 400 mg, 450 mg, 500 mg, 550 mg, 600 mg, 750 mg, and 1,000 mg being exemplary doses.

The treatment can be administered, for example, 1, 2, 3, 4, or 5 times daily, weekly, bi-weekly, etc., for 1, 2, 3, 4, or more weeks, and for example until symptoms improve or disappear, or other biochemical or physiological endpoint is reached. In some embodiments, a single treatment can be repeated 1, 2, 3, 4, 5, 6, 7, or more days, weeks, or months apart. In some embodiments, the treatment period is for days, weeks, or months. For example, in some embodiments, the treatment period is between 1-62 days, or any subrange or specific integer number of days therebetween, inclusive. In non-limiting examples, the treatment period is 5 days, 7 days, 10 days, 14 days, 15 days, 20 days, 21 days, 28 days, 30 days, 31 days, 40 days, 50 days, 56 days, 58 days, 60 days, 61 days, or 62 days long.

In some embodiments, the treatment regimen is similar to those describe above for, e.g., gout, prolonging penicillin serum levels, pelvic inflammatory disease, gonorrhea, etc.

In a particular embodiments, the probenecid or a metabolite or analog or prodrug thereof or pharmaceutically acceptable salt thereof is administered as 250 mg twice per day.

Results show that 2 mg/kg and 200 mg/kg dosages were both effective at treating SARS-CoV-2 in hamsters in vivo. Thus, in some embodiments, the dosage is between 2 mg/kg and 200 mg/kg, inclusive.

Results below also show that pop-PK model-generated probenecid exposure profiles show that at steady state 500 mg bid, 600 mg bid, 900 mg bid, 1000 mg bid and 1800 mg qd dosing regimens would lead to achieving concentration multifold higher than drug level required for 90% inhibition of viral replication. These doses are all below the maximum allowable FDA-approved dose and are generally safe with no significant side effects.

Thus, in some embodiments, the dosage is between 500 mg and 2,000 mg, or between 600 mg and 1,800 mg, one or twice daily. The results in the Examples below indicate that dosage for many viruses except flu can be in the 500-1000 mg once or twice daily range. The dose for flu is likely to be lower because the IC50 is in the picomolar range Specific dosages are, for example, 500 mg or 1000 mg twice daily. Dosage many also be determined based on weight, particularly in pediatric patients.

As introduced above, recitation of ranges of values herein including the dosage ranges above and elsewhere herein, are merely intended to serve as a shorthand method of referring individually to each separate value falling within the range and each separate value is incorporated into the specification as if it were individually recited herein.

Dosing regimens may be, for example, intermittent dosing or continuous (e.g., constant infusion). The dosing regimens can include administrations of the same or different doses. Thus, the dosing regimen can include dose escalation, dose reduction, or a combination thereof.

In some embodiments, the composition is administered in a pulsed dosage regimen. Pulse dosing refers to dosing approach that produces escalating drug levels early in the dosing interval followed by a prolonged dose-free period. For example, in some embodiments, drug administration is frontloaded by means of, for example, 1, 2, 3, 4, or 5 sequential bolus administrations, after which drug levels are allowed to diminish until the next dose. In some embodiments, the serum drug level is allowed to diminish to about 0.

This type of drug delivery technology could offer therapeutic advantages such as reduced dose frequency and greater patient compliance. In comparison to intermittent dosing, pulse dosing front loads the drug, allowing an extended dose-free period during which drug concentration falls close to zero. However, unlike a single, large bolus dose (e.g., given once daily), short bursts of drug are separated by short dose-free periods, allowing the serum concentration to fluctuate (Ibrahim, et al., Antimicrobial Agents and Chemotherapy, 48(11):4195-4199 (2004)). In particular embodiments, pulse dosing is carried out by oral administration or intravenous administration. For example, in some embodiments, the therapy includes discontinuous/intermittent intravenous infusion of very high doses of probenecid, a metabolite or analog or prodrug thereof, or a pharmaceutically acceptable salt thereof over a short period.

In some embodiments, a large bolus dose of probenecid, a metabolite or analog or prodrug thereof, or pharmaceutically acceptable salt thereof is between about 1,000 mg and 5,000 mg inclusive, or any subrange or specific dosage there between.

In some embodiments, a single dose including (e.g., one or two injections) provides adequate plasma concentrations for either treatment or prophylactic concentrations over the entire treatment period. In some embodiments, the treatment period is 1-31 days, or any integer number of days therebetween. Such a long-acting formulation may be time-release or slow release formulation, such as time-release or slow-release parenteral formulation.

In some embodiments, probenecid, metabolite, analog, or prodrug is administered a dosage known in the art. The maximum recommended dosage for probenecid is 2 grams/day PO for adults, adolescents, and children of more than 50 kg, and 40 mg/kg/day (1.2 grams/m2/day) PO (not to exceed 2 grams/day PO) for adolescents and children of 50 kg or less. Thus, in some embodiments, administration does not exceed 5 g, 4 g, 3 g, or 2 g per day. In some embodiments, administration does not exceed 40 mg/kg/day. See also “probenecid—Drug Summary”, the Prescribers' Digital Reference.

In some embodiments, a tablet for oral administration contains e.g., 500 mg of probenecid and optionally, one or more of the following inactive ingredients: microcrystalline cellulose, sodium lauryl sulfate, sodium starch glycolate, starch (corn), povidone, colloidal silicon dioxide, magnesium stearate, polyvinyl alcohol, titanium dioxide, polyethylene glycol, talc, D&C Yellow #10 Aluminum Lake, FD&C Yellow #6 Aluminum Lake, and FD&C Blue #2 Aluminum Lake.

In some embodiments, a subject is administered 500 mg of probenecid or a metabolite or analog or prodrug thereof, or pharmaceutically acceptable salt thereof, orally (P.O.) twice daily (B.I.D.) for 14 days.

In some embodiments, treatment ends 0, 1, 2, 3, 4, or 5 days after the subject's symptom(s) resolve, the subject registers one, two, or more negative tests for viral infections (e.g., negative SARS-CoV-2/COVID-19 tests), or a combination thereof.

In some embodiments, the dose and/or dosage regimen is effective to achieve a plasma concentration in the subject that is or exceeds IC90, optionally but preferably over a 5-10 day period. This is believed to be sufficient for both treatment and prophylaxis. See also, Example 4 below.

The results presented in the Examples below illustrate strain differences in drug efficacy that may relate to the replication rate of the virus and/or cell tropism as viruses prefer certain cell types in which to replicate. For example flu A appears more sensitive than flu B to probenecid and the same is true for RSV A vs RSV B. Such results indicate that a lower dosage and/or less frequent dosage regimen may be effective for treating more sensitive viruses, and relatively higher dosage and/or more frequent dosage regiment may be needed for treating less sensitive viruses.

Dosages in non-animals can the same or similar to those used in humans, adjusted as has been done for other drugs, or determined empirically. For example, doses and bioavailability in pigs may be similar to humans as they also have a similar metabolism. See, e.g., Nielson, et al., The American Journal of Clinical Nutrition, Volume 99, Issue 4, April 2014, Pages 941-949, doi.org/10.3945/ajcn.113.074724; Tang and Mayersohn, Drug Metabolism and Disposition, 46 (11) 1712-1724; doi.org/10.1124/dmd.118.083311; and Hutchinson, et al., Phil. Trans. R. Soc. B369:20130583.20130583, doi.org/10.1098/rstb.2013.0583

### F. Combination Therapies

In some embodiments, probenecid, a metabolite or analog or prodrug thereof, or a pharmaceutically acceptable salt thereof is administered in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of probenecid, a metabolite or analog or prodrug thereof, or a pharmaceutically acceptable salt thereof.

In some embodiments, the combination of the probenecid, a metabolite or analog or prodrug thereof, or a pharmaceutically acceptable salt thereof and a second or more active agent(s) leads to an additive or more than additive response in the subject in need thereof.

In some embodiments, the second active agent is an antiviral (i.e., a second antiviral), a fever reducer, an anti-inflammatory, an analgesic, or a combination thereof.

Some of the drugs that can be used as a second drug for treating infections include but are not limited to Oxaprozin, Ketorolac Tromethamine, Irbesartan, Balsalazide, Meclofenamic Acid, Nateglinide, Diflunisal, Valsartan, Ethacrynic acid, Pioglitazone, Amlexanox, Nitazoxanide, Telmisartan, Ivosidenib, Lenvatinib, Teriflunomide, Taurocholic acid, Salicylic acid, Quinidine, Benzylpenicillin, Ouabain, Indomethacin, Ibuprofen, Guanidine, Glutaric Acid, Furosemide, Diclofenac, Cholic Acid, Bumetanide, Cilastatin, Piroxicam, Aminohippuric acid, Caprylic acid, Cimetidine, Aspartame, Tetracycline, Oxytetracycline, Minocycline, Ganciclovir, Acyclovir, Dinoprostone, Cefalotin, Cefoperazone, Cefazolin, Cefamandole, Cefadroxil, Ceftriaxone, Cefotaxime, Phenylbutazone, Ketoprofen, Famotidine, Liothyronine, Methotrexate, Conjugated estrogens, Tenoxicam, Enalapril, trans-2-hydroxycinnamic acid, Cephalexin, Valproic acid, Melatonin, Benzoic acid, Mercaptopurine, Novobiocin, Liotrix, Cefacetrile, Zidovudine, Dabrafenib, Topiroxostat, Ataluren, Enasidenib, Letermovir, Dolutegravir, Rucaparib, Baricitinib, Apalutamide, Avatrombopag, Cefaclor, Cefotiam, Ceftibuten, Ceftizoxime, Cefaloridine, Leucovorin, Rosuvastatin, Ivermectin, Rifampicin, Cabotegravir, Pradigastat, Lansoprazole, Acetylsalicylic acid, Pantoprazole, Esomeprazole, Pravastatin, Tazobactam, Pretomanid, Latanoprost, Gemfibrozil, Dronedarone, Tafamidis, Rimegepant, Favipiravir, Osilodrostat, Hydroflumethiazide, Artesunate, Ritonavir, Lopinavir, and Losartan.

Exemplary anti-inflammatory drugs that can be included in the pharmaceutical composition or pharmaceutical formulation include, but are not limited to, ibuprofen, naproxen sodium, aspirin, naproxen sodium, diclofenac potassium, celecoxib, sulindac, oxaprozin, piroxicam, indomethacin, meloxicam, fenoprofen, naproxen, esomeprazole, diclofenac, diflunisal, etodolac, ketorolac tromethamine, ketoprofen, meclofenamate, nabumetone, salsalate, tolmetin, and steroids, such as corticosteroids (e.g. hydrocortisone, cortisone, ethamethasoneb, prednisone, prednisolone, triamcinolone, methylprednisolone, and dexamethasone) and mineralocorticoids (e.g. fludrocortisone), and a combination thereof.

Exemplary antiviral drugs that can be included in the pharmaceutical composition or pharmaceutical formulation include, but are not limited to, anti-SARS-CoV-2 monoclonal or polyclonal antibodies, convalescent plasma (e.g., from a subject previously diagnosed with COVID-19), itaconate, Acyclovir, Adefovir, Amantadine, Ampligen, Umifenovir, Baloxavir marboxil, Biktarvy, Boceprivir, Bulevirtide, Combivir, Daclastavir, Darunavir, Delavirdine, Descovy, Didanosine, Docosanol, Dolutegravir, Doravirine, Edoxudine, Ensitrelvir, Famciclovir, Foscarnet, Ganciclovir, Ibacitabine, Idoxuridine, Imiquimod, Imunovir, Indinavir, Letermovir, Methisazone, Moroxydine, Nexavir, Nitazoxanide, Oseltamivir, Penciclovir, Peramavir, Pleconaril, Podophyllotoxin, Remdesivir, Ribavirin, Rilpivirine, Rimantadine, Simeprevir, Sofosbuvir, Taribavirin, Telaprivir, Telbivudine, Tenofovir alafenamide, Tipranavir, Tromantadine, Umifenovir, Valaciclovir, Valganciclovir, Vidarabine, Zalcitabine, Zanamivir, Zidovudine, Nirmatrelvir, Remdesivir, Molnupiravir, interferon alpha, interferon beta, interferon lambda, ivermectin, hydroxychloroquine, chloroquine, and fluvoxamine.

In a particular embodiment, the antiviral is oseltamivir phosphate (TAMIFLU®). Tamiflu is a prescription medicine used to treat the flu (influenza) in people 2 weeks of age and older who have had flu symptoms. Probenecid or metabolites or analogs or prodrug or pharmaceutical salts thereof may enhance the efficacy of antivirals like oseltamivir phosphate as it helps retain excretion of the drug during treatment.

Some products that may interact with the disclosed compounds include: cancer chemotherapy, baricitinib, dyphylline, ketorolac, methotrexate, pyrazinamide, salicylates (e.g., high-dose aspirin), zidovudine, certain drugs removed from the body by the kidneys (such as ceftazidime/avibactam, dapsone, heparin, fosfomycin). Thus, in some embodiments, the subject is not administered one or more of these drugs while being treated with probenecid or metabolites or analogs or prodrugs or pharmaceutical salts thereof.

In some embodiments, one or more additional active agent is remdesivir.

The disclosed invention can be further understood by the following numbered paragraphs.

1. A compound having the structure of:

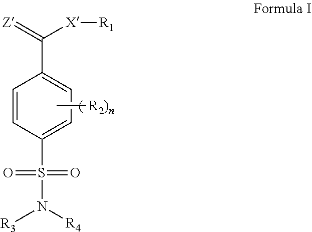

wherein:
(a) Z′ is O, NR₅, or S;
(b) X′ is absent, O, NR₅, or S;
(c) R₁ is hydrogen, a substituted or unsubstituted alkyl, a substituted or unsubstituted alkenyl, a substituted or unsubstituted alkynyl, a substituted or unsubstituted aryl, a substituted or unsubstituted heteroaryl, a substituted or unsubstituted polyaryl, a substituted or unsubstituted polyheteroaryl, an azo, an alkoxy, a polyether, a thiol, a sulfanimine, an amino, a carbonate, an ester, an amide, a carbamate, an imine, a substituted or unsubstituted carbonyl, a hydroxyl, a polyol, a phosphonyl, sulfinyl, a sulfonamide, a nitro, a cyano, a lipid, a peptide, a cholesterol, a phytosterol, a glycoside, or a glucuronide;
(d) n is an integer from 0 to 4;
(e) each R₂ is independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted aralkyl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen;
(f) R₃-R₅ are independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol; and
(g) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

2. The compound of paragraph 1, wherein R₁ is hydrogen, a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, a substituted or unsubstituted heteropolyaryl, a polyol, a polyalkylene glycol, a lipid, a peptide, a cholesterol, a phytosterol, a glucuronide,

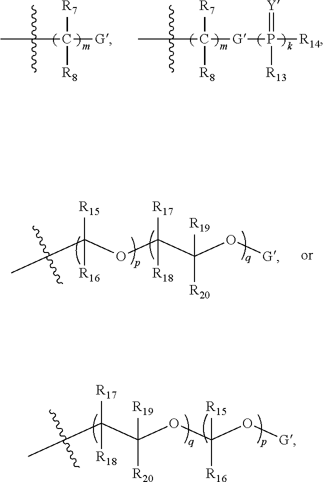

wherein G′ is hydrogen, a lipid, a peptide, a cholesterol, a phytosterol, a glycoside, a glucuronide,

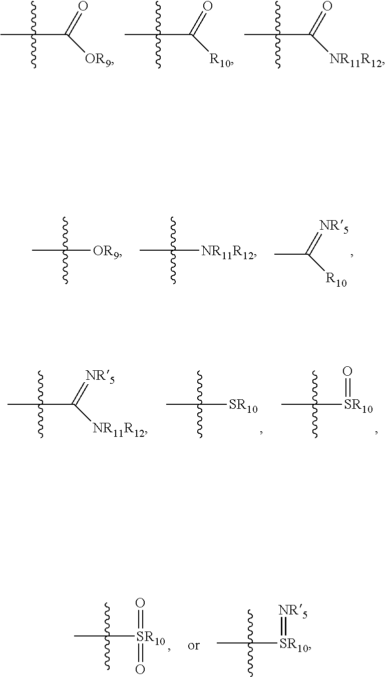

and
R₉-R₁₂ are independently hydrogen, a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, an alkoxy, a di-alkyl amino, or a halogen;
R′₅ is independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol, such as a hydrogen or a substituted or unsubstituted C₁-C₆ alkyl (e.g., an unsubstituted C₁-C₆ linear or branched alkyl, an unsubstituted C₁-C₆ cycloalkyl, an unsubstituted C₁-C₄ linear or branched alkyl, an unsubstituted C₁-C₄ cycloalkyl, an unsubstituted C₁-C₃ linear or branched alkyl, an unsubstituted C₁-C₃ cycloalkyl, etc.);
m, k, p, and q are independently an integer from 0 to 20, from 0 to 18, from 0 to 16, from 0 to 14, from 0 to 12, from 0 to 10, from 0 to 8, from 0 to 6, from 0 to 4, from 0 to 3, or from 0 to 2, such as 0 or 1;
each Y′ is independently O or S;
each occurrence of R₇, R₈, and R₁₅-R₂₀ is independently hydrogen, a substituted or unsubstituted C₁-C₂₀ alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen, or
R₇ and R₈ together, R₁₅ and R₁₆ together, and/or R₁₇ and R₁₈ together, with the carbon atom to which they are attached, form a C₁-C₂₀ cycloalkyl, or
when X′ is NR₅, m is not 0, at least one of p and q is not 0, then (i) R₇ is hydrogen and R₈ is a substituted or unsubstituted C₁-C₂₀ alkyl that form a ring together with R₅ that includes the adjoining N and C atoms, (ii) R₁₅ is hydrogen and R₁₆ is a substituted or unsubstituted C₁-C₂₀ alkyl that form a ring together with R₅ that includes the adjoining N and C atoms, and/or (iii) R₁₇ is hydrogen and R₁₈ is a substituted or unsubstituted C₁-C₂₀ alkyl that form a ring together with R₅ that includes the adjoining N and C atoms; and
R₁₃ and R₁₄ are independently hydrogen, a substituted or unsubstituted C₁-C₂₀ alkyl, or an alkoxy.

3. The compound of paragraph 2, wherein R₁ is an unsubstituted C₁-C₂₀ linear or branched alkyl, an unsubstituted C₃-C₂₀ cycloalkyl, a C₁-C₂₀ haloalkyl, an unsubstituted aryl, an unsubstituted polyaryl, an unsubstituted heteroaryl, an unsubstituted heteropolyaryl, a polyalkylene glycol, a lipid, a peptide, a cholesterol, a phytosterol, a glucuronide,

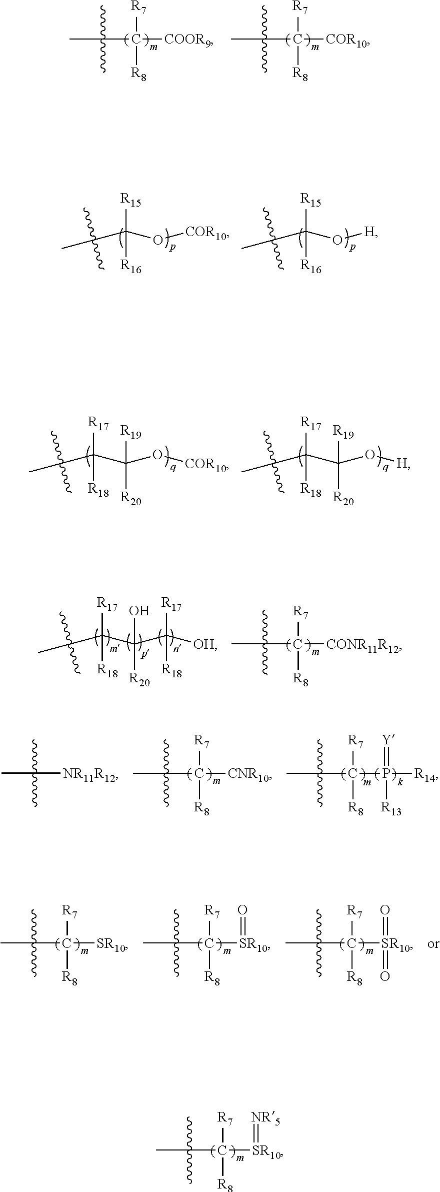

and wherein m, m′, p′, and n′ are independently an integer from 0 to 10, from 0 to 8, from 0 to 6, from 0 to 4, from 0 to 3, from 0 to 2, or 0 or 1, p is an integer from 1 to 6, from 1 to 4, from 1 to 3, or 1 or 2, and k is an integer from 1 to 6, from 1 to 4, or.

4. The compound of any one of paragraphs 1-3, wherein Z′ is O, X′ is absent or O, and R₁ is a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, a substituted or unsubstituted heteropolyaryl, a polyalkylene glycol, a lipid, a peptide, a cholesterol, a phytosterol, a glycoside, or a glucuronide.

5. The compound of any one of paragraphs 1-3, wherein Z′ is O or R₅ and

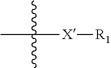

is

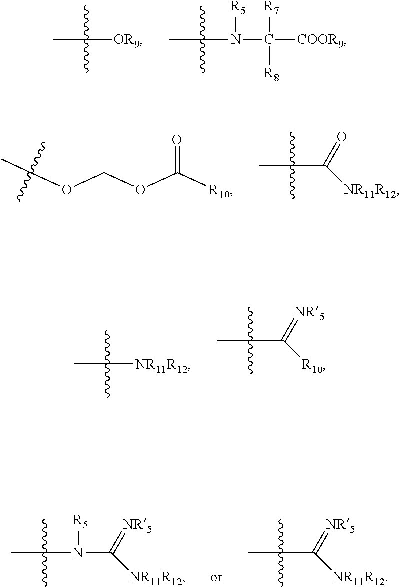

6. The compound of any one of paragraphs 1-3, wherein Z′ is O, X′ is O, and R₁ is

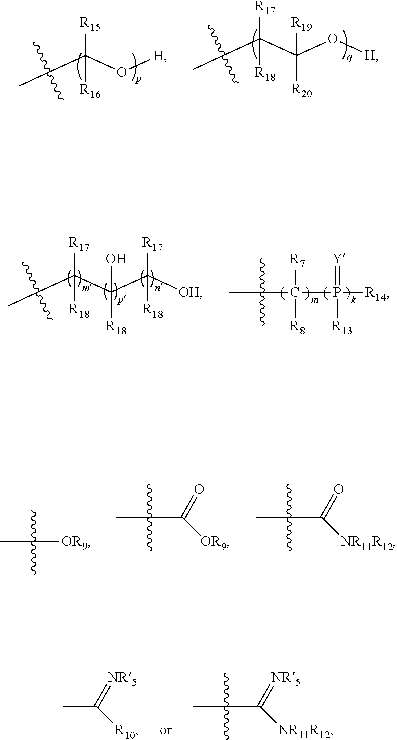

optionally wherein Z′ is O, X′ can be O, and R₁ is

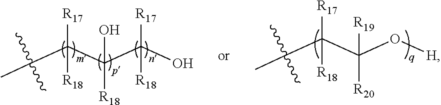

m′ and n′ are independently an integer from 0 to 6, from 0 to 5, from 0 to 4, from 0 to 3, from 1 to 3, such as 1 or 2; p′ is an integer from 1 to 10, from 1 to 8, from 1 to 6, from 1 to 4, or from 1 to 3; q is an integer from 1 to 10, from 1 to 8, from 1 to 6, from 1 to 4, or from 1 to 3; and each occurrence of R₁₇-R₂₀ are independently hydrogen, hydroxyl, —SH, —(CH₂)₁₋₆NR₂₂R₂₃, —(CH₂)₁₋₆OH, —(CH₂)₁₋₆SH, or an unsubstituted C₁-C₁₀ alkyl.

7. The compound of any one of paragraphs 1-3, wherein Z′ is O, X′ is S, and R₁ is

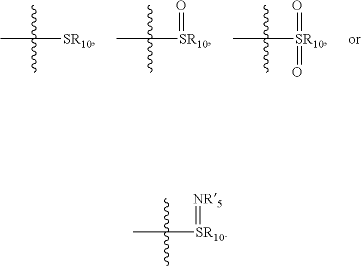

8. The compound of any one of paragraphs 2-7, wherein each occurrence of R₇, R₈, and R₁₅-R₂₀ is independently hydrogen, a substituted or unsubstituted C₁-C₂₀ alkyl, —(CH₂)₁₋₆NR₂₂R₂₃, —(CH₂)₁₋₆OH, a substituted or unsubstituted aralkyl, —(CH₂)₁₋₆SH, —(CH₂)₁₋₆S(O)₀₋₂CH₃, —(CH₂)₁₋₆NHC(═NH)NH₂, -(1H-indol-3-yl) methyl, -(1H-imidazol-4-yl)methyl, —(CH₂)₀₋₆COOR₂₁, —(CH₂)₀₋₆CONR₂₂R₂₃, a substituted or unsubstituted aryl, an aryl-C₁₋₃ alkyl, CH₂-indol-3-yl, —(CH₂)₁₋₆SCH₃, —CH₂-imidazol-4-yl, CH(OH)(CH₂)₀₋₅CH₃, —CH₂((4′-OH)-Ph), and wherein R₂₁-R₂₃ are independently hydrogen or an unsubstituted C₁₋₆ alkyl.

9. The compound of any one of paragraphs 1-8, wherein R₃ and R₄ are independently hydrogen, a substituted or unsubstituted alkyl, a substituted or unsubstituted aryl, a substituted or unsubstituted hetercyclyl, a substituted or unsubstituted heteroaryl, an alkoxy, an amino, or an imine, preferably R₃ and R₄ are independently hydrogen or a substituted or unsubstituted C₁-C₂₀ alkyl such as an unsubstituted methyl, ethyl, propyl, butyl, pentyl, or hexyl, for example, an unsubstituted propyl; and/or wherein each occurrence of R₂ is independently hydrogen, hydroxyl, —SH, —(CH₂)₁₋₆NR₂₂R₂₃, —(CH₂)₁₋₆OH, —(CH₂)₁₋₆SH, or an unsubstituted C₁-C₁₀ alkyl, such as hydrogen.

10. The compound of any one of paragraphs 1-9, wherein R₅ and/or R′₅ is(are) independently hydrogen or a substituted or unsubstituted C₁-C₂₀ alkyl.

11. The compound of any one of paragraphs 1-10, wherein when substituents are present, the substituents are independently an unsubstituted C₁-C₆ alkyl, a C₁-C₆ alkyl substituted with unsubstituted C₁₋₆ alkyl, an unsubstituted C₁-C₆ heteroalkyl, a C₁-C₆ heteroalkyl substituted with unsubstituted C₁₋₆ alkyl, an unsubstituted C₂-C₆ alkenyl, an unsubstituted C₂-C₆ alkynyl, an unsubstituted aryl, an unsubstituted heteroaryl, an unsubstituted C₁-C₆ alkoxy, —(CH₂)₁₋₆CO₂R₂₁, a halogen, C₁-C₆ haloalkyl, —NR₂₂R₂₃, C₁₋₆ acylamino, —NHSO₂C₁₋₆ alkyl, —SO₂NR₂₂R₂₃, —SO₂C₁₋₆ alkyl, —COOR₂₁, —CONR₂₂R₂₃, nitro, cyano, hydroxide, thiol, or an aryl or heteroaryl substituted with unsubstituted C₁_₅ alkyl, an alkoxy, a di(C₁₋₆ alkyl)-amino, a fluoro, or an unsubstituted C₃-C₆ cycloalkyl.

12. The compound of any one of paragraphs 2-11, wherein the peptide comprises or is a peptide selected from RKKRRQRRR (SEQ ID NO:6), RRRRRRRR (SEQ ID NO:7), RKKRRRESRKKRRRES (SEQ ID NO:8), GRPRESGKKRKRKRLKP (SEQ ID NO:9), RQIKIWFQNRRMKWKK (SEQ ID NO:10), GRRRRRRRRRPPQ (SEQ ID NO:11), LLIILRRRIRKQAHAHSK (SEQ ID NO:12), RVRVFVVHIPRLT (SEQ ID NO:13), GALFLGFLGAAGSTMGAWSQPKKKRVK (SEQ ID NO:14), KLALKLALKALKAALKLA (SEQ ID NO:15), GWTLNSAGYLLGKINLKALAALAKKIL (SEQ ID NO:16), VSALK (SEQ ID NO:17), CSIPPEVKFNPFVYLI (SEQ ID NO:18), GIGAVLKVLTTGLPALISWIKRKRQQ (SEQ ID NO:19), HGLASTLTRWAHYNALIRAF (SEQ ID NO:20).

13. The compound of any one of paragraphs 1-12, wherein the compound is not any one of the compounds in Table 2, optionally wherein the compound is

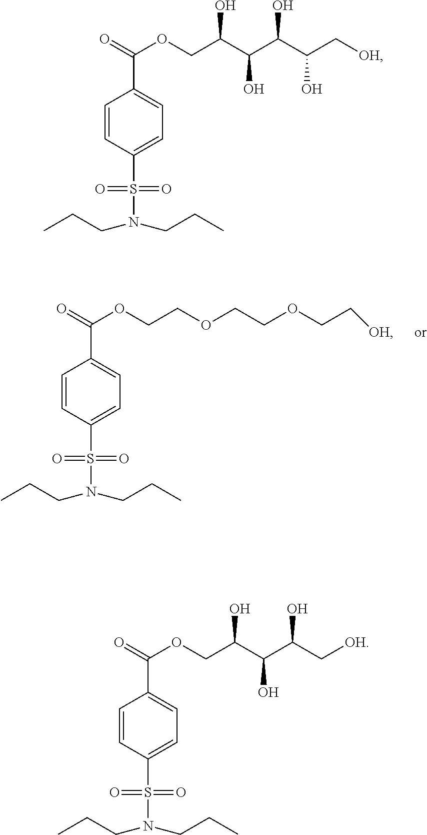 [SMILES: CCCN(CCC)S(=O)(=O)c1ccc(C(=O)OC[C@@H](O)[C@H](O)[C@@H](O)[C@@H](O)CO)cc1 ; CCCN(CCC)S(=O)(=O)c1ccc(C(=O)OCCOCCOCCO)cc1 ; "or" ; CCCN(CCC)S(=O)(=O)c1ccc(C(=O)OC[C@@H](O)[C@H](O)[C@@H](O)CO)cc1]

14. A pharmaceutical formulation comprising: one or more compounds of any one of paragraphs 1-13; and a pharmaceutically acceptable excipient and/or carrier, wherein the one or more compounds are in an effective amount to prevent, treat, or ameliorate one or more symptoms associated with a viral infection in a subject in need thereof.

15. The pharmaceutical formulation of paragraph 14, wherein the pharmaceutically acceptable carrier is nanoparticles, liposomes, cyclodextrins, or hydrogels, and optionally wherein the one or more prodrugs are encapsulated in, conjugated to, and/or complexed with the nanoparticles, liposomes, cyclodextrins, or hydrogels.

16. The pharmaceutical formulation of paragraph 14 or 15, wherein the pharmaceutical formulation is in the form of tablets, syrups, capsules, powders, or microneedles.

17. The pharmaceutical formulation of any one of paragraphs 14-16, further comprising one or more additional active agents, and optionally wherein the one or more additional active agents is/are one or more antiviral and/or anti-inflammatory agents.

18. A method of treating or preventing a viral infection in a subject comprising administering the subject an effective amount of the compound of any one of paragraphs 1-13.

19. A method of treating or preventing a viral infection comprising administering each of the subjects of the group an effective amount of a compound selected from probenecid, or a metabolite or analog, or prodrug thereof, or pharmaceutically acceptable salt thereof, optionally wherein the prodrug is a compound of any one of paragraphs 1-13, optionally wherein the group comprises at least one subject exposed to a subject with the viral infection.

20. The method of paragraph 19, wherein the subjects are infected with the virus, not infected with the virus, or a combination thereof.

21. The method of any one of paragraphs 18-20, wherein the wherein the viral infection is due to DNA or RNA viruses.

22. The method of paragraph 21, wherein the viral infection is due to DNA viruses belonging to the family adenoviridae, papoviridae, herpesviridae, poxviridae, anelloviridae or pleolipoviridae.

23. The method of paragraph 21, wherein the viral infection is due to RNA viruses belonging to the family reoviridae, picornaviridae, caliciviridae, togaviridae, arenaviridae, Flaviviridae, Orthomyxoviridae, paramyxoviridae, bunyaviridae, rhabdoviridae, filoviridae, coronaviridae, astroviridae, bornaviridae, arteriviridae, Nymaviridae, Pneumoviridae, Flaviviridae, Hepeviridae/Nodaviridae, Picornaviridae, or Togaviridae.

24. The method of any one of paragraphs 21-23, wherein the virus is a respiratory virus.

25. The method of any one of paragraphs 21-23, wherein the virus is selected from influenza viruses, optionally influenza virus A, influenza virus B, or influenza virus C, respiratory syncytial virus (RSV), human metapneumovirus, coronaviruses, measles virus, parainfluenza virus, mumps virus, Zika virus, dengue virus, yellow fever virus, Japanese encephalitis virus, West Nile virus, Hepatitis A virus, Hepatitis B virus, or Hepatitis C virus.

26. The method of paragraph 20, wherein the virus is a coronavirus selected from a Severe acute respiratory syndrome-related coronavirus, a Bat Hp-betacoronavirus Zhejiang2013, a Rousettus bat coronavirus GCCDCl, a Rousettus bat coronavirus HKU9, a Eidolon bat coronavirus C704, a Pipistrellus bat coronavirus HKU5, a Tylonycteris bar coronovirus HKU4, a Middle East respiratory syndrome-related coronavirus, a Hedgehog coronavirus, a murine coronavirus, a Human coronavirus HKU1, a China Rattus coronavirus HKU24, a Betacoronavirus 1, a Myodes coronavirus 2JL14, a Human coronavirus NL63, a Human coronavirus 229E, and a Human coronavirus OC43.

27. The method of paragraph 26, wherein the coronavirus is a Severe acute respiratory syndrome-related coronavirus.

28. The method of paragraph 27, wherein the Severe acute respiratory syndrome-related coronavirus is SARS-CoV-2, SARS-CoV, SARSr-CoV RaTG13, SARS-CoV PC4-227, or SARSr-CoV BtKY72.

29. The method of paragraph 28, wherein the Severe acute respiratory syndrome-related coronavirus is SARS-CoV-2, optionally within the subject has COVID 19.

30. A method of treating or preventing a viral infection in a subject comprising administering the subject an effective amount of a compound selected from probenecid, or a metabolite or analog, or prodrug thereof, or pharmaceutically acceptable salt thereof, optionally wherein the prodrug is a compound of any one of paragraphs 1-13, wherein the virus causing the infection is selected from Zika virus, dengue virus, RSV subtype A, and RSV subtype B.

31. The method of any one of paragraphs 18-30, wherein the virus has an RNA genome optionally encoding an RNA-dependent RNA polymerase (RdRp), optionally is a member of the kingdom Orthornavirae, optionally utilizes a host organic anion transporter optionally selected from OAT1, OAT2, OAT3, OAT4, OAT5, OAT6, OAT7, rOAT8, OAT9, OAT10, and/or URAT1.

32. The method of any one of paragraphs 18-31, wherein the subject has one or more symptoms are selected from fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhea, tiredness, nausea, anosmia, skin rash, and combinations thereof.

33. The method of any one of paragraphs 18-31, wherein the subject is asymptomatic.

34. The method of any one of paragraphs 18-33, wherein compound is in a delivery vehicle optionally selected from nanoparticles and liposomes.

35. The method of any one of paragraphs 18-34, wherein compound is in a pharmaceutical composition further comprising a pharmaceutically acceptable carrier and/or excipient.

36. The method of any one of paragraphs 18-35, wherein the compound is administered systemically.

37. The method any one of paragraphs 18-36, wherein the compound is administered orally, parenterally, topically, or mucosally.

38. The method of any one of paragraphs 18-37, wherein the compound is administered mucosally to the lungs, nasal mucosa, or combination thereof.

39. The method of any one of paragraphs 18-38, wherein the compound is administered in an effective amount to reduce viral replication.

40. The method of any one of paragraphs 18-39, wherein the compound is at a dosage of 10 mg-2,000 mg, or 600 mg, 900 mg, or 1,800 mg, optionally twice daily, optionally for 14 days.

41. The method of any one of paragraphs 18-40, wherein the subject is treated by pulse dosing.

42. The method of any one of paragraphs 18-41, wherein the subject is a human.

43. The method of any one of paragraphs 18-42, wherein the compound is administered in a dose of 250 mg to 2,000 mg once or twice a day, optionally wherein the dose is 600 mg or 900 mg twice a day, or 1,800 mg once a day.

44. The method of any one of paragraphs 18-42, wherein the compound is administered to the subject for two weeks or more.

45. The method of any one of paragraphs 18-44, wherein the subject or subjects is/are human(s), non-human mammal(s), or bird(s).

46. The method of any one of paragraphs 18-45, wherein the subject or subjects is/are non-human mammal(s) or bird(s), and wherein the compound is formulated in the subject(s)'s drinking water, milk, or feed, and administered when the subject drinks the water or eats the feed.

47. The method of paragraph 46, wherein the subject(s) is/are chicken(s), optionally wherein the virus is influenza A H5N1.

48. The method of paragraph 46, wherein the subject(s) is/are pig(s), optionally wherein the virus is influenza A H1N1.

49. The method of any one of paragraphs 18-45, wherein the subject or subjects is/are human and the virus is measles.

50. The method of paragraph 49, wherein the subject or subjects is/are pediatric subjects, optionally between the ages as of 2-10 inclusive.

51. An animal feed comprising an effective amount of a compound selected from probenecid, or a metabolite or analog, or prodrug thereof, or pharmaceutically acceptable salt thereof, optionally wherein the prodrug is a compound of any one of paragraphs 1-13.

52. The animal feed of paragraph 51 further comprising one or more of crude proteins, fats, sugars, amino acids, minerals, starch, and vitamins.

53. A method of treating a subject for gout comprising administering a subject in need thereof an effective amount of the compound of any one of paragraphs 1-13.

54. A method of treating a subject for hyperuricaemia comprising administering a subject in need thereof an effective amount of the compound of any one of paragraphs 1-13.

### EXAMPLES

### Example 1: Probenecid Prophylactically and Therapeutically Reduces RSV Replication

Respiratory syncytial virus (RSV) is the leading viral pathogen associated with lower respiratory tract disease in infants and young children worldwide also afflicting the elderly and immune-compromised (Welliver, et al., Curr Med Res Opin 26, 2175-2181, doi:10.1185/03007995.2010.505126 (2010), Falsey, et al., The New England Journal of Medicine 352, 1749-1759, doi:10.1056/NEJMoa043951 (2005)). Preventing RSV morbidity and mortality has been an effort of research and vaccine studies development for decades. RSV is responsible for >150,000 pediatric hospitalizations/year costing >$300 million in young children (Han, et al., J Infect Dis 179, 25-30, doi:10.1086/314567 (1999)). Therapeutic intervention is limited to inhaled ribavirin and palivizumab (Synagis), a humanized monoclonal antibody targeting the F protein. Ribavirin has shown mixed-to-poor results and palivizumab treatment is not fully effective (Turner, et al., Clinicoecon Outcomes Res 6, 217-225, doi:10.2147/CEOR.S60710 (2014), Foolad, et al., Clin Infect Dis 68, 1641-1649, doi:10.1093/cid/ciy760 (2019)). Additionally, palivizumab is administered monthly to help protect high-risk infants from severe RSV disease throughout the RSV season, and although treatment reduces hospitalizations in treated infants by approximately 50%, its efficacy decreases as mutations in F protein are induced by treatment (Olchanski, et al., Open Forum Infect Dis 5, ofy031, doi:10.1093/ofid/ofy031 (2018), Moore, et al., J Pediatr 214, 121-127 e121, doi:10.1016/j.jpeds.2019.06.058 (2019). Unfortunately, there is no safe and effective RSV vaccine available despite years of effort, thus there is a need for effective RSV therapeutics.

### Materials and Methods

Murray, et al., “Probenecid Inhibits Respiratory Syncytial Virus (RSV) Replication,” Viruses, 14(5):912 (2022). doi: 10.3390/v14050912; and Murray, et al., “Probenecid Inhibits Respiratory Syncytial Virus (RSV) Replication,” Research Square, posted Feb. 1, 2022, doi.org/10.21203/rs.3.rs-1280404/v1, are specifically incorporated by reference herein in their entireties.

### Cells and Cell Culture

Vero E6 cells (ATCC; CRL-1586) and human epithelial (HEp-2) cells (ATCC; CCL-23) were propagated in Dulbecco's modified Eagle's medium (DMEM; Gibco) supplemented with 5% heat-inactivated fetal bovine serum (FBS; Hyclone) at 37° C. with 5% CO₂. Vero E6 cells and HEp-2 cells were maintained in log-phase in T75 cm² culture flasks (ThermoFisher) and HEp-2 was used for virus propagation. HEp-2 and Vero E6 cells depend largely on RSV G protein binding to cell surface glycosaminoglycans (GAGs). GAG-dependent infection is reduced by a single passage of RSV in Vero E6 cells (Kwilas, et al., J Virol 83, 10710-10718, doi:10.1128/JVI.00986-09 (2009)). Normal human bronchial epithelial (NHBE) cells (Lonza) from a healthy male donor were expanded, cryopreserved, and maintained in bronchial epithelial cell growth medium (BEGM; Lonza) through 15 population doublings and were used undifferentiated.

### Viruses

RSV A2 (ATCC VR-1540), RSV B1 (ATCC VR-1580), or Memphis-37 (a clinical strain of human RSV strain A obtained from Meridian Life Science) were propagated and quantified on HEp-2 cells and Vero E6 cells then stored at −80° C. as described previously (Haynes, et al., J Virol 76, 6873-6881 (2002)). HEp-2 cells and Vero E6 cells were maintained in Dulbecco's modified essential medium (DMEM) supplemented with glutamine and 5% fetal bovine serum (5% DMEM; Gibco). Virus titers were determined using a methylcellulose plaque assay as described (Matrosovich, et al., Virol J 3, 63, doi:10.1186/1743-422X-3-63 (2006)).

### In Vitro Probenecid Inhibition Assays

A working stock of probenecid (Sigma) was dissolved in DMSO (Sigma) and dilutions of the working stock were resuspended in PBS (Gibco). Cellular toxicity was determined using a ToxiLight Bioassay (Lonza). Vero E6 cells, HEp-2 cells, or Undifferentiated NHBE cells were plated overnight at 10⁴ cells/well in 96-well flat-bottom plates (Costar). Cells were either pretreated for 24 h prior to infection (prophylactically) or therapeutically at 24 h post-infection with probenecid at different concentrations, i.e. 100, 50, 25, 12, 6, 3, 1, 0.5, 0.2, 0.1, 0.05, 0.01, or 0 μM. Subsequently, the media and probenecid were removed and the cells were infected with RSV A2, RSV B1, or Memphis-37 at an MOI=0.1. At 72 h post-infection the plates containing the cells were frozen at −80° C. the freeze-thawed 3× and the cell-free supernatants were used for log 10 dilutions in RSV plaque assays.

### In Vivo Inhibition Studies

BALB/c male and female mice (6-8 weeks old) were obtained from Charles River and rested a week before use. All experiments and procedures were approved by the Institutional Animal Care and Use Committee (IACUC) of the University of Georgia. All experiments were performed with five mice per group and repeated twice independently. To evaluate lung virus titers, probenecid was administered intraperitoneally (i.p.) at doses and time points pre- or post-RSV infection as indicated in the Results. Briefly, 2 mg/kg or 200 mg/kg of probenecid in PBS were i.p. delivered to the mice. On days 3, 5, and 7 bronchoalveolar lavage (BAL) samples were collected from individual mice and analyzed. BAL cell yield was determined by counting the total cell number, and cell viability was determined by Trypan blue (Sigma) exclusion. Smears for cell differentiation were prepared by cytocentrifugation (Shandon), and cell differentiation was performed by microscopy on cytospun slides after staining with hematoxylin and eosin staining where at least 100 cells were counted for macrophages, polymorphonuclear (PMN) cells, lymphocytes, and eosinophils (Haynes, et al., J Virol 77, 9831-9844, doi:10.1128/jvi.77.18.9831-9844.2003 (2003)). At each time point, sera were collected, and the lungs were isolated to determine virus titers by PFU/ml analyses (Perwitasari, et al., Antimicrob Agents Chemother 57, 475-483, doi:10.1128/AAC.01532-12 (2013)). For virus titration analyses, lung homogenates were serially diluted, and the titer was determined on Vero E6 cells (Caidi, et al., Antiviral Res 154, 149-157, doi:10.1016/j.antiviral.2018.04.014 (2018)).

The BAL cell pattern reflects the inflammatory cell profile in the lung (Haynes, et al., J Virol 77, 9831-9844, doi:10.1128/jvi.77.18.9831-9844.2003 (2003)). Neither prophylactic nor therapeutic probenecid treatment with 2 mg/kg or 200 mg/kg probenecid had substantial effects on the differential cell counts or BAL leukocyte subpopulations at days 3, 5, or 7 pi (Table 5). Further, no substantial differences in BAL cells were evident by smears despite the reduced RSV lung titers in the probenecid-treated mice highlighting the anti-RSV effects of the drug.

### Lung Virus Titers

Lung viral titers from RSV-infected mice were determined as previously described (Haynes, et al., J Virol 76, 6873-6881 (2002)). Briefly, lungs were homogenized in 1 ml of sterile Dulbecco PBS per lung, and 10-fold serial dilutions in serum-free DMEM (Gibco) were added to confluent Vero cell monolayers in 24-well plates. After adsorption for 2 h at 37° C., cell monolayers were overlaid with 2% methylcellulose, incubated at 37° C. for 6 days, and then enumerated by immunostaining with anti-F protein monoclonal antibody, 131-2A.

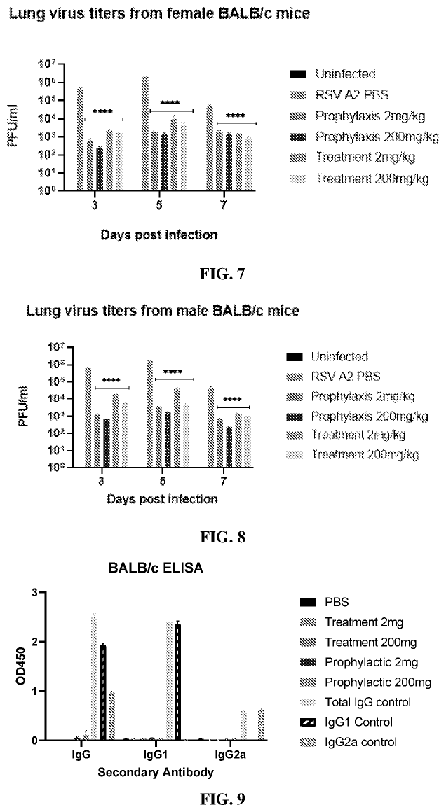

RSV-specific ELISA Antibodies against RSV prevent disease by various mechanisms including virus neutralization, antibody-dependent cellular cytotoxicity (ADCC), and complement-mediated neutralization. To determine if probenecid treatment affected the anti-RSV antibody response, the sera from four female mice per group collected at days 7 pi were diluted (1:40) and assayed by ELISA using a modified protocol as described (Bergeron, et al., Viruses 13, doi:10.3390/v13020352 (2021)). The ELISA detects both neutralizing and non-neutralizing antibodies and the use of RSV A2 lysate antigen provides a way to detect antibodies against multiple RSV proteins. As expected, there were very low IgG, IgG1, and IgG2a levels as these mice received a primary infection and sera were collected 7d post-challenge (FIG. 9).

### OAT3 Expression

SLC22A8 (OAT3) transcripts were evaluated by qPCR as previously described (Perwitasari, et al., Pharmaceuticals (Basel) 6, 124-160, doi:10.3390/ph6020124 (2013), Tripp, et al., Methods Mol Biol 555, 43-61, doi:10.1007/978-1-60327-295-7_4 (2009), Wu, et al., Sci Data 4, 170021, doi:10.1038/sdata.2017.21 (2017)). For in vitro studies, HEp-2 cells were plated in 96-well tissue culture plates (Corning) and treated with the IC90 of probenecid [7.2 uM] or DMSO only control for 24 h. RNA was isolated by RNAzol RT (Molecular Research Center) and digested with DNAse1, and total RNA was quantified by Nanodrop (ThermoFisher). cDNA first-strand synthesis was performed using LunaScript (New England Biolabs) as described by the manufacturer. cDNA was used as a template for qPCR in Luna Universal qPCR master mix (New England Biolabs). For in vivo studies, BALB/c lung RNA was extracted by RNAdvance Tissue (Beckman Coulter) at indicated time points and processed as described above.

TABLE 3
Primer pairs used.

| Gene | Primer Left (5′-3′) | Primer Right (5′-3′) |
|---|---|---|
| SLC22A8 | TGCAAATGAATGCGAATGAGG | CGGTCGTCGCATAACACATA |
| (huOAT3) | (SEQ ID NO: 21) | (SEQ ID NO: 25) |
| ACTB | CATGTACGTTGCTATCCAGGC | CTCCTTAATGTCACGCACGAT |
| (huB-actin) | (SEQ ID NO: 22) | (SEQ ID NO: 26) |
| SLC22A8 | CATACTCACTCCTGCACTCATC | CCAGGGAATCTCAAAGGGAAA |
| (msOAT3) | (SEQ ID NO: 23) | (SEQ ID NO: 27) |
| ACTB | CAGCCTTCCTTCTTGGGTATG | GGCATAGAGGTCTTTACGGATG |
| (msB-actin) | (SEQ ID NO: 24) | (SEQ ID NO: 28) |

Gene expression was determined, and raw Ct values or fold change (reciprocal of 2ΔΔCt) are presented normalized to a housekeeping gene. Data represent mean Ct values ± 95% confidence interval, or SEM, respectively, of three independent repeats.

### Statistical Analysis.

Statistical analyses were done using the Student's t-test or one-way analysis of variance (ANOVA), as indicated. Results were calculated as means±standard errors. Values of p<0.05 were considered significant.

### Results

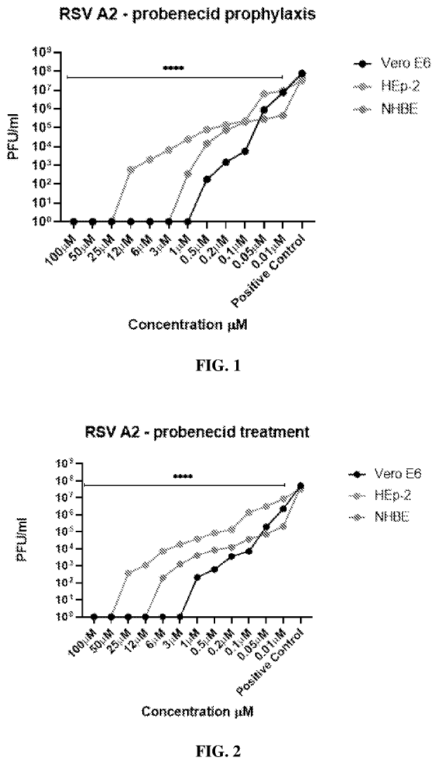

Experiments were designed to determined if RSV replication in Vero E6 cells, HEp-2 cells, or NHBE cells infected with RSV A2, RSV B1, or Memphis-37 was affected by probenecid treatment. The different epithelial cell types were pretreated (prophylaxis) with differing probenecid concentrations (i.e., 100, 50, 25, 12, 6, 3, 1, 0.5, 0.2, 0.1, 0.05, 0.01, or 0 μM) and the effect of treatment on replication determined at 72 h after infection by plaque assay. Probenecid prophylaxis resulted in a dose-dependent decrease in RSV A2 replication in all infected cells types with an IC50/IC90=0.07/0.63 uM in Vero E6 cells, 0.8/7.2 uM in HEp-2 cells, and 0.4/3.6 uM in NHBE cells (FIG. 1).

Cell viability was examined and no cellular toxicity was evidently similar to earlier studies (Perwitasari, et al., Antimicrob Agents Chemother 57, 475-483, doi:10.1128/AAC.01532-12 (2013), Murray, et al., Sci Rep 11, 18085, doi:10.1038/s41598-021-97658-w (2021)). Moreover, HEp-2 cells treated with IC90 probenecid resulted in undetectable levels of OAT3 transcripts (Table 4).

TABLE 4
OAT3 Expression.

| | Probenecid | |
|---|---|---|
| | Treated | DMSO only |
| HEp-2 Cells | Raw Ct Value | Raw Ct Value |
| ACTB | 16.3 (95% CI 14.8- | 15.3 (95% CI 14.4- |
| | 17.8) | 16.2) |
| OAT3 | No Ct | 32.1 (95% CI 31.2- |
| | | 33) |

| | |
|---|---|
| BALB/c Mice | Probenecid Treated (200 mg/kg) |
| | Fold change reduction |
| OAT3 | 15.6 (95% CI 3.8-27.4) |

HEp-2 cells were treated with IC₉₀ concentration of probenecid [7.2 uM] or mock-treated (DMSO only) for 24 h. OAT3 transcripts were determined as described in Methods. Since probenecid treatment resulted in undetectable levels of OAT3 transcripts, fold-change was not performed. Lung RNA was extracted at day 2 pi in mice treated with 200 mg/kg probenecid or PBS (i.e., 24 h post-treatment). OAT3 gene expression in 200 mg/kg probenecid treated mice was normalized to a housekeeping gene, and compared to PBS treated mice. The data is represented as the reciprocal of 2^{ΔΔct} (fold-change decrease). Data represent the mean Ct values or fold-change of three individual experiments and 95% confidence intervals (CI).

Probenecid treatment was very effective at inhibiting RSV A2 replication in all cells types (FIG. 2). The IC50/IC90=0.1/2.7 uM in Vero E6 cells, 1.2/10.8 uM in HEp-2 cells, and 0.3/2.7 uM in NHBE cells. The results for probenecid prophylaxis showed the highest IC50/IC90 activity in Vero E6 cells and NHBE cells.

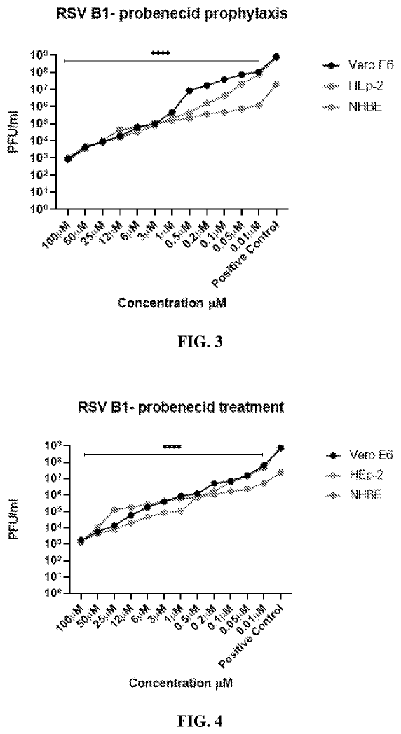

As RSV groups A and B co-circulate, and both groups may cause infection during a single season (Sullender, et al., Clin Microbiol Rev 13, 1-15, table of contents, doi:10.1128/CMR.13.1.1 (2000), it was important to determine the probenecid susceptibility to RSV A and RSV B particularly as it has been shown that the two groups have evolved separately for a considerable period (Mufson, et al., J Gen Virol 66 (Pt 10), 2111-2124, doi:10.1099/0022-1317-66-10-2111 (1985)). As for RSV A2, probenecid prophylaxis resulted in a dose-dependent decrease in RSV B1 replication in all infected cells types (FIG. 3). There was no IC90 for RSV B1 in the treated cell types because RSV B1 was not reduced 90% using the concentrations tested. Probenecid prophylaxis resulted in an IC50=0.85 uM in Vero E6 cells, 0.8 uM in HEp-2 cells, and 0.8 uM in NHBE cells (FIG. 3, Table 5). Probenecid treated Vero E6 cells infected with RSV B1 had an IC50=2.0 uM, HEp-2 cells=0.9 uM, and NHBE cells=1.2 uM (FIG. 4, Table 5). Similar to RSV A2 infected cells there was no cellular toxicity detected. The results showed that probenecid prophylaxis or treatment was more effective for RSV A2 infected cell types compared to RSV B1.

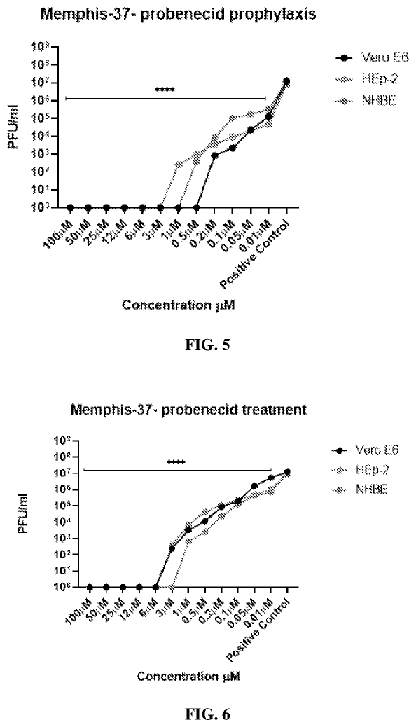

Memphis-37 is an RSV A strain isolated from a pediatric case and used in studies in human adult subjects (Kim, et al., PLoS One 9, e113100, doi:10.1371/journal.pone.0113100 (2014)). Memphis-37 that is propagated in Vero E6 cells have been shown to develop a truncated G protein (Kwilas, et al. J Virol 83, 10710-10718, doi:10.1128/JVI.00986-09 (2009)), thus the Memphis-37 strain used in these studies was propagated in HEp-2 cells. Probenecid prophylaxis was effective at inhibiting Memphis-37 replication in all infected cells types (FIG. 5). The IC50/IC90=0.03/0.27 uM in Vero E6 cells, 0.04/0.36 uM in HEp-2 cells, and 0.16/1.44 uM in NHBE cells (Table 5), and no effect on cell viability was detectable for any probenecid concentration. Treatment with probenecid inhibited Memphis-37 replication in all infected cells types as expected and was similar to RSV A2 and B1 studies (FIG. 6). The IC50/IC90=0.4/3.6 uM in Vero E6 cells, 0.5/4.5 uM in HEp-2 cells, and 0.2/1.8 uM in NHBE cells (Table 5).

TABLE 5
IC50/IC90 values.

| | | IC50 | | | IC90 | | |
|---|---|---|---|---|---|---|---|
| | | RSV A2 | RSV B1 | Memphis-37 | RSV A2 | RSV B1 | Memphis-37 |
| Prophylaxis | Vero E6 cells | 0.07 μM | 0.85 μM | 0.03 μM | 0.63 μM | * | 0.27 μM |
| | HEp-2 cells | 0.8 μM | 0.8 μM | 0.04 μM | 7.2 μM | * | 0.36 μM |
| | NHBE cells | 0.4 μM | 0.8 μM | 0.16 μM | 3.6 μM | * | 1.44 μM |
| Treatment | Vero E6 cells | 0.1 μM | 2.0 μM | 0.4 μM | 2.7 μM | * | 3.6 μM |
| | HEp-2 cells | 1.2 μM | 0.9 μM | 0.5 μM | 10.8 μM | * | 4.5 μM |
| | NHBE cells | 0.3 μM | 1.2 μM | 0.2 μM | 2.7 μM | * | 1.8 μM |

Table Legend. IC50 and IC90 values in NHBE cells, Vero E6 cells, and HEp-2 cells after treating with different probenecid concentrations and infecting with RSV A2, RSV B1, or Memphis-37.
* = no IC90value for the RSV B1 virus as there was not a 90% reduction of virus titers with the concentrations of probenecid used.

Having shown probenecid to have potent activity on prophylactically or therapeutically treated cell types (FIGS. 1-6), the effectiveness of prophylactic or therapeutic treatment in a BALB/c mouse model of RSV infection was tested. Male or female 6-8-week-old BALB/c mice were intranasally (i.n.) infected with RSV strain A2. Mice were treated once with probenecid 24 h before infection (prophylaxis) or 24 h post-infection (treatment) dosed at 2 mg/kg or 200 mg/kg, or with PBS. There were no substantial clinical signs of disease determined by BAL cell infiltrates (Tables 6 and 7), and treatment did not affect antibody levels (FIG. 9) (Altamirano-Lagos, et al., Front Microbiol 10, 873, doi:10.3389/fmicb.2019.00873 (2019)).

TABLE 6
BAL cells/mL.

| | | BAL | | |
|---|---|---|---|---|
| | | cells/mL | | |
| | Group | Day 3 | Day 5 | Day 7 |
| Males | Prophylactic 2 mg/kg | 125000 | 118750 | 90625 |
| | Prophylactic 200 mg/kg | 243750 | 293750 | 65625 |
| | Treatment 2 mg/kg | 193750 | 120000 | 87500 |
| | Treatment 200 mg/kg | 265000 | 145000 | 96875 |
| | PBS | 243750 | 100000 | 97500 |
| Females | Prophylactic 2 mg/kg | 268750 | 256250 | 75000 |
| | Prophylactic 200 mg/kg | 308333.333 | 280000 | 125000 |
| | Treatment 2 mg/kg | 360000 | 270000 | 135000 |
| | Treatment 200 mg/kg | 312500 | 140000 | 91666.6667 |
| | PBS | 235000 | 170000 | 250000 |

TABLE 7
BAL cell types.

| | | Total Macrophages | | | Total Lymphocytes | | | Total PMNs | | | Total Eosinophils | | |
|---|---|---|---|---|---|---|---|---|---|---|---|---|---|
| | | Day 3 | Day 5 | Day 7 | Day 3 | Day 5 | Day 7 | Day 3 | Day 5 | Day 7 | Day 3 | Day 5 | Day 7 |
| Males | Prophylactic 2 mg/kg | 19300 | 21530 | 16610 | 360 | 390 | 675 | 260 | 0 | 0 | 80 | 80 | 30 |
| | Prophylactic 200 mg/kg | 70460 | 45280 | 12675 | 1380 | 1000 | 206 | 1080 | 370 | 0 | 80 | 350 | 44 |
| | Treatment 2 mg/kg | 30260 | 23180 | 16625 | 630 | 310 | 430 | 0 | 380 | 0 | 110 | 130 | 100 |
| | Treatment 200 mg/kg | 33730 | 28070 | 13985 | 770 | 290 | 1155 | 500 | 470 | 0 | 0 | 170 | 55 |
| | RSV A2 PBS | 29220 | 19230 | 18625 | 280 | 460 | 630 | 110 | 210 | 0 | 390 | 100 | 20 |
| Females | Prophylactic 2 mg/kg | 51050 | 51760 | 13590 | 1350 | 1800 | 250 | 600 | 280 | 130 | 0 | 160 | 30 |
| | Prophylactic 200 mg/kg | 51400 | 54890 | 24160 | 1400 | 850 | 260 | 200 | 90 | 370 | 333 | 170 | 210 |
| | Treatment 2 mg/kg | 68950 | 52220 | 26380 | 2380 | 1450 | 400 | 130 | 0 | 100 | 540 | 330 | 120 |
| | Treatment 200 mg/kg | 28538 | 24370 | 18133 | 600 | 370 | 200 | 563 | 60 | 0 | 300 | 200 | 0 |
| | RSV A2 PBS | 45340 | 32510 | 48900 | 940 | 1060 | 610 | 610 | 300 | 110 | 110 | 130 | 380 |

BAL cells were collected from BALB/c mice at days 3, 5, or 7 post-infection. Total cell number and cell viability were determined by Trypan blue exclusion. Neither prophylactic nor therapeutic probenecid treatment with 2 mg/kg or 200 mg/kg probenecid had significant effects on the cell counts or BAL leukocyte subpopulations at days 3, 5, or 7 pi.

All probenecid regimens had significantly (p<0.0001) reduced lung virus titer on days 3, 5, and 7 pi in female and male mice (FIGS. 7 and 8, respectively). As predicted from the in vitro results (FIGS. 1 and 2), there was a considerable reduction in the lung virus load in 2 mg/kg and 200 mg/kg probenecid-treated mice challenged with RSV A2. Maximum reductions of lung virus load occurred in mice pretreated with 200 mg/kg probenecid 24 h before infection although substantial reductions in lung virus titer occurred following 2 mg/kg probenecid prophylaxis (FIGS. 7 and 8). Mice therapeutically treated once with 2 or 200 mg/kg probenecid 24 h after RSV infection also had greatly reduced RSV A2 lung titers on days 3, 5, and 7 pi (FIGS. 7 and 8). Maximum reductions of lung virus load occurred in mice treated with 200 mg/kg probenecid, although significant (p<0.0001) and substantial reductions in lung virus titers were observed in 2 mg/kg probenecid-treated mice. Moreover, RNA extracted from the lungs of mice treated with 200 mg/kg probenecid had markedly reduced OAT3 transcripts compared to PBS controls 2 dpi (Table 4).

As previously reported (Murray, et al., Sci Rep 11, 18085, doi:10.1038/s41598-021-97658-w (2021), which is specifically incorporated by reference in its entirety), a population pharmacokinetics (pop-PK) model was developed to characterize probenecid PK using a one-compartment structure with saturable elimination and first-order absorption. Simulations using the final pop-PK model to generate probenecid exposure profiles comparing 600 mg twice daily, 900 mg twice daily, or 1800 mg once daily administration was completed and free drug concentrations were calculated (Table 8).

TABLE 8
Probenecid steady-state concentration and free drug
concentrations after different probenecid doses.

| Dose | | Steady state concentration | Steady-state concentration (mM) with 95% |
|---|---|---|---|
| (mg) | Frequency | (mg/ml) | protein binding |
| 600 | BID | 30.1 | 5.27 |
| 900 | BID | 92.5 | 16.2 |
| 1800 | QD | 64.9 | 11.4 |

The doses examined are predicted to provide plasma concentrations exceeding the protein binding adjusted IC50/IC90 values for all RSV strains under all study conditions. The projected doses are below the maximum allowable FDA-approved dose and have been generally safe and well-tolerated with no significant side effects.

In sum, probenecid pretreatment of Vero E6 cells, HEp-2 cells, or NHBE cells was very effective at preventing RSV replication. The IC50 and IC90 of probenecid prophylaxis against RSV A2 was IC50/IC90=0.07/0.63 uM in Vero E6 cells, 0.8/7.2 uM in HEp-2 cells, and 0.4/3.6 uM in NHBE cells. Similarly, the IC50 of probenecid treatment of RSV B1 infected Vero E6 cells was IC50=0.85 uM, 0.8 uM for HEp-2 cells, and 0.8 uM for NHBE cells. Importantly, comparable IC50/IC90 results following probenecid prophylaxis or treatment of Memphis-37 infected cells were evident. These results show that nanomolar concentrations of probenecid reduce RSV virus replication. Importantly, probenecid administered prophylactically before RSV infection resulted in reduced lung virus titers in vivo. Likewise, probenecid given therapeutically at 24 h post-RSV infection also resulted in reduced lung virus titers demonstrating the versatility of probenecid as a chemotherapeutic. Importantly, human plasma concentrations for probenecid are projected to exceed the protein binding adjusted IC50/IC90 value over the dosing interval providing adequate coverage against the tested strains.

### Example 2: Probenecid Therapeutically Reduces Replication of Mumps, Dengue, and Zika

### Materials and Methods

### Mumps, Zika, Dengue Assays

### Experiments Conducted in Vero-P Cells.

Cells were plated in 96-well plate and treated the next day with varying concentrations of probenecid.

After 24 cells were infected with virus (MOI=0.1) for 1 hour Inoculum was decanted and replaced with media containing varying concentrations of probenecid.

Plates were left for 3 days before being frozen at −80 C until plaquing in Vero-P cells.

### Influenza Assays

### Experiment Conducted in A549 Cells.

Cells were plated in 96-well plate and treated the next day with varying concentrations of Probenecid.

After 24 cells were infected with influenza virus (MOI=0.1) for 1 hour

Inoculum was decanted and replaced with media containing varying concentrations of probenecid.

Plates were left for 3 days before being frozen at −80 C until plaquing in MDCK cells.

The virus is plaqued in MDCK cells while studies are carried out in human respiratory epithelial cells.

### Results

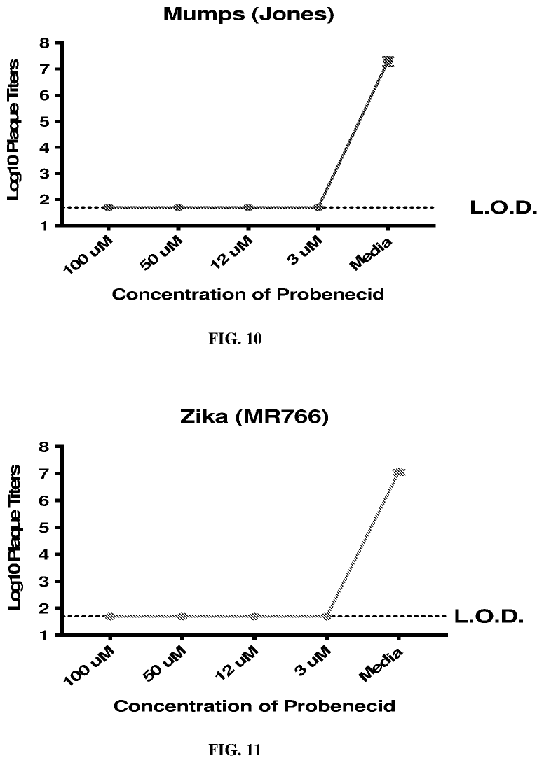

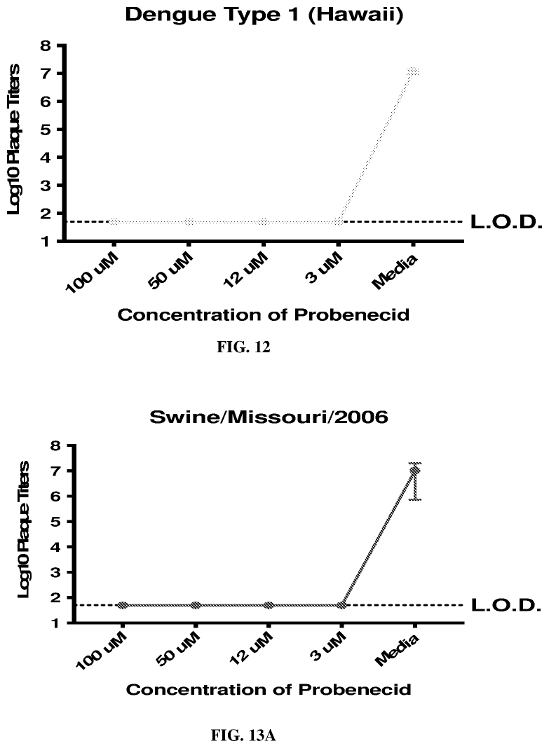

Experiments were designed to test the ability of probenecid to treat infection of Mumps, Zika, and Dengue viruses. The results are presented in FIGS. 10-12, respectively, and show that picomolar levels of probenecid were effective at inhibiting virus replication.

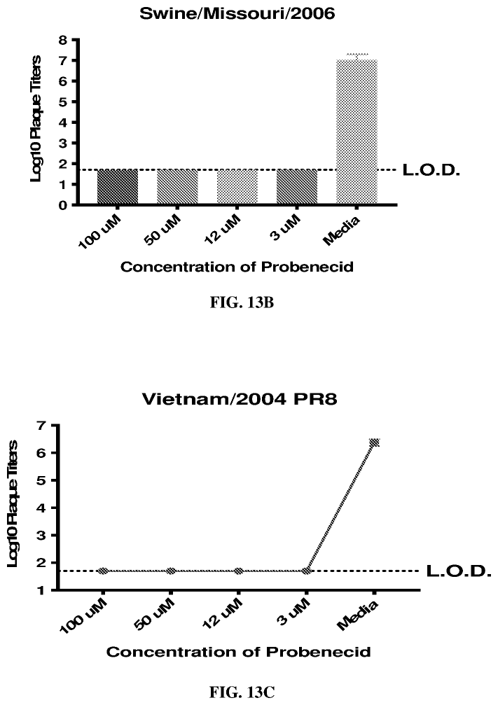

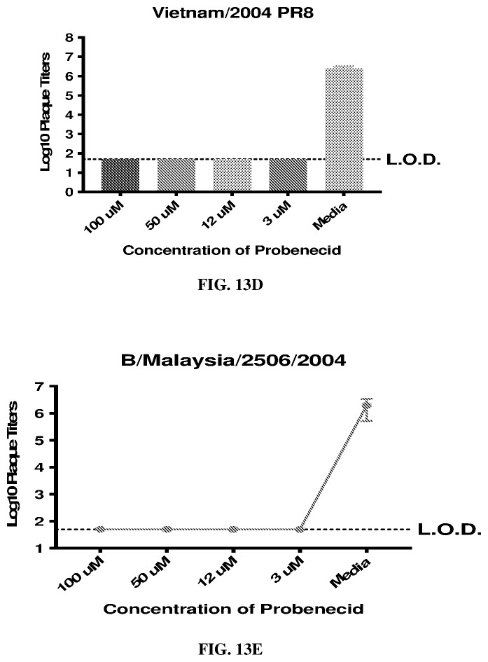

Results are also presented for three strains of influenza: influenza A strain Swine/Missouri/2006 (FIGS. 13A, 13B), influenza A strain Vietnam/2004 PR8 (FIGS. 13C, 13D), and influenza B strain B/Malaysia/2506/2004 (FIGS. 13E, 13F).

Experiments were also designed to test if probenecid treatment induces virus resistance, by comparing probenecid therapeutic treatment of A549 cells. A/WSN/33 infected A549 cells were treated with 0, 10⁻⁴, 10⁻³, 10⁻², 10⁻¹, 100, 10, and 1 μM probenecid for 72 h and assessed and virus replication by plaque assay. After treatment, any virus was attempted to be passaged and assessed for growth. No detectable virus replication was identified. The experiment was repeated twice with the same result.

### Example 3: Probenecid Prophylactically and Therapeutically Reduces Measles Virus Infection

### Materials and Methods

Plate 4×10\hat{ }4/well of Vero-E6 or Hep-2 cells to be 95-100% confluent the next day. The next day make fresh dilutions of Probenecid were made. From a stock concentration of 10 mM in 100% DMSO, dilutions were made with half DMSO and half DMEM with 4% BSA fraction. FBS inhibits some viruses. Media was decanted and dilutions of probenecid were added to cells. Controls: DMSO/Media mixture, Media with no Probenecid, Media with no virus. Probenecid dilutions were left on cells for 24 hrs. After 24 hrs, media was decanted and virus was added (MOI=0.01/well). Virus was diluted to MOI=0.01 per 100 uL with DMEM. Left virus inoculum on cells for 1 hr. After 1 hr, infection media was decanted and replaced with dilutions of Probenecid. For Immunostaining, plates were left for 24 hrs before fixing cells. For Plaque Assays, plates were left for 72 hrs before freezing at −80 C.

The immunostaining protocol generally included fixing cells with 4% formaldehyde in PBS (50 uL) for 15-20 min at room temp, rinsing cells 3× with PBS (100 uL), and optionally storing cells at 4° C. in PBS until ready to stain. Permeabilizing cells with 0.05% TritonX-100 in PBS (100 uL) for 10 min at room temp, rinsing cells 3× with PBS (100 uL), blocking with 3% BSA-PBS (100 uL) for 1 hr at 37° C. or overnight at 4° C. Blocking solution was removed and cells were incubated with primary antibody (mouse mAb uNP, Millipore MAB8906) at 1:10,000 dilution (100 uL) for 1 hour (antibody diluted in 3% BSA-PBS). Cells were washed 3× with KPL Buffer (100 uL), incubated for 45 min with secondary antibody (Goat uMouse conjugated with Alex flour 488) in the dark at 37° C. (antibody diluted in 3% BSA-PBS). Cells were washed 3× with KPL Buffer in the dark (100 uL), rapidly stained with DAPI (1 ug/mL) for 10 min in the dark, washed 3× with PBS in the dark and stored in 200 ul of PBS until visualization.

For plaque assays of probenecid treated cells, cells were treated with various concentrations of Probenecid before being infected with Measles virus (MOI=0.01). After 72 hours, cells were frozen until plaque assay. Plaque concentrations are recorded as log 10 PFU/mL.

### Results

Experiments were designed to test the prophylactic effect of probenecid on measles virus infection. Cells were infected with measle virus with, or without, probenecid treatment and immunostained for syncytia. Syncytia occurs when multiple cells fuse forming a large multinucleated cell which eventually goes on to die forming a hole or plaque in the cell lawn. In an exemplary experiment, a Control Well (24 hrs): Media only and no probenecid (NP fluorescence stain), showed 520 syncytia. In an Experimental Well treated with 1 uM Probenecid (NP fluorescence stain), no syncytia were detected.

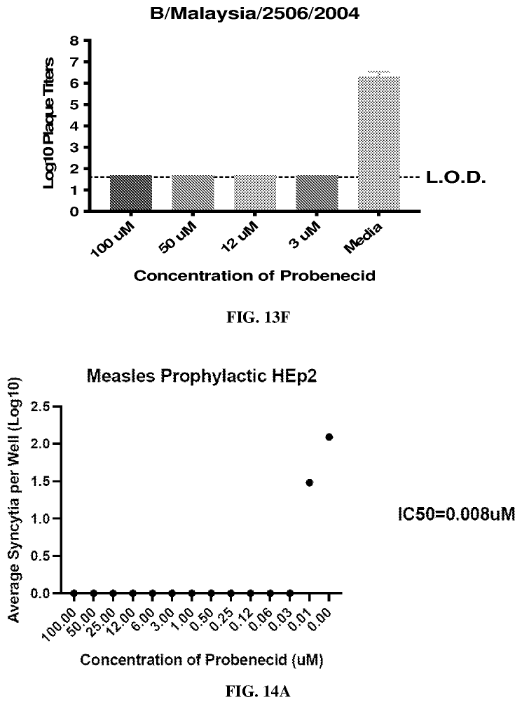

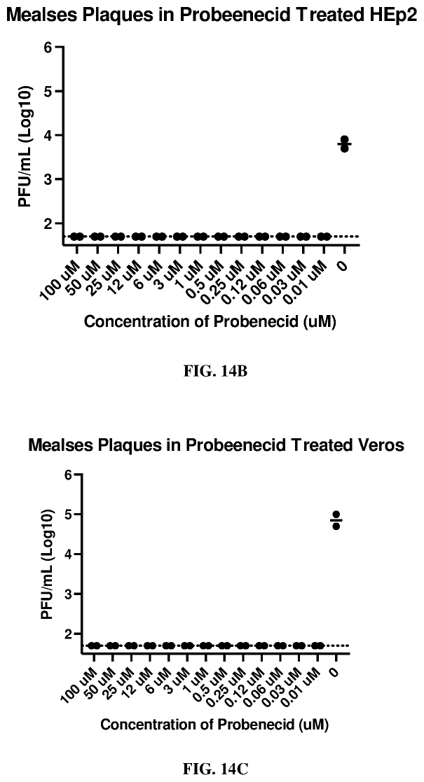

Results of a probenecid dilution assay show an IC50=0.0008 uM, leading to the conclusion that MeV is very sensitive to probenecid. See FIG. 14A showing average syncytia per well (Log 10) relative to the concentration of probenecid. In other assays, 0.03 UM was also effective on MeV in Hep-2 cells.

In plaque assays cells were treated with various concentrations of probenecid before being infected with Measles virus (MOI=0.01). All plaque titers were below the limit of detection for the Probenecid treated HEp2 and Vero cells. See FIGS. 14B and 14C. These results are representative of more than three independent studies, all showing the same trend i.e. very high drug efficacy in Vero and Hep-2 cells.

### Example 4: Modeling and Simulation of Probenecid Pharmacokinetics (PK)

In in vitro and pre-clinical hamster studies, probenecid was shown to block replication of the SARS-CoV-2 virus and the associated acute inflammatory response to the infection (WO 2021/207606 and Murray, et al., Sci Rep 11, 18085 (2021), doi.org/10.1038/s41598-021-97658-w, which are specifically incorporated by reference herein in their entireties). In an open label, ten patients, twenty-eight (28) day, Investigator Initiated Study (IIS) in non-hospitalized patients with mild to moderate SARS-CoV-2 infection were treated with probenecid. Although a scored grading system was not used, patient symptoms showed improvement. Coagulation and inflammatory markers were mostly maintained within normal ranges with the exception of one patient, who upon enrollment had several elevated biomarkers. See, e.g., WO 2021/207606.

In this Example, a probenecid concentration time plot, following oral administration to healthy adult male volunteers were digitized. The digitized data were then used to develop a population PK model using the Phoenix NLME software platform. The final PK model was then used to simulate a variety of potential treatment regimens, and the resulting predicted exposures were then compared with free probenecid concentration needed to achieve 90% inhibition of COVID-19 viral replication (IC90) at time of infection within 0.2 to 0.25 hr based on the initial dose.

Data obtained by digitization represented the mean value for 5 subjects. These data were fit in a nonlinear mixed effects framework using Phoenix NLME software (Javed, et al., Front. Plant. Sci., 11:601335 (2020). doi: 10.3389/fpls.2020.601335). Parameter estimates reported by Selen et al for a PK model with saturating (Michaelis-Menten) elimination were used as initial estimates for this current analysis (Selen et al., J. Pharm. Sci. 71:1238-1242 (1982) doi: 10.1002/jps.2600711114). The model was fitted using first order conditional estimation method with extended least square (FOCE-ELS) algorithm. The base model testing included basic one and two-compartment structures with first order absorption. Other absorption, lag time and clearance models were also evaluated. As the available data was summary level (i.e. mean values), estimates of between-subject variability were not estimable or meaningful for this analysis.

An additive plus proportional residual error model was initially included in the base models, but other forms of residual error models were tested to improve stability/fit. A battery of diagnostic plots was employed to evaluate the adequacy of the goodness-of-fit for the final PK model. The key plots consisted of the following:
Observed individual concentration (DV) versus population predictions (PRED) or DV versus individual predictions (IPRED), evaluated for random scatter around the line of identity;
CWRES versus PRED and CWRES versus time after dose (TAD), evaluated for random scatter around the horizontal line across zero.

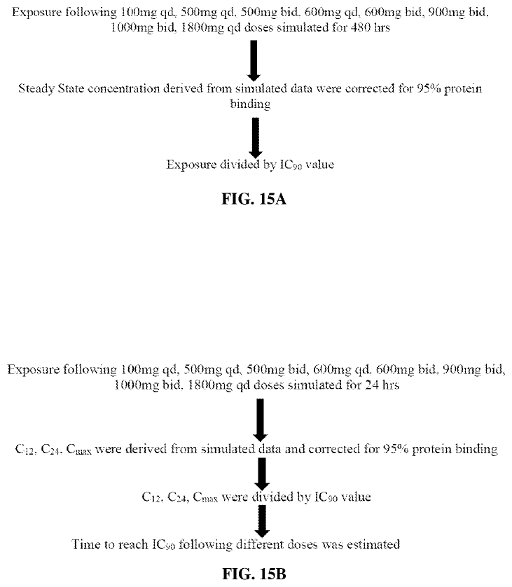

Additionally, plots of DV, IPRED versus TAD were used to assess the model fit. The final pop PK model obtained after the initial and final model building steps was used to simulate expected exposures to explore different dosing regimens. The aim of the simulation was to estimate free steady-state exposures following different regimens and time to reach IC90 concentration. The IC90 level is 0.36369 μM (Investigator Brochure) and the molecular weight of probenecid is 285.36 g/mol. The simulation analysis is detailed in flow-chart in FIGS. 15A-15B.

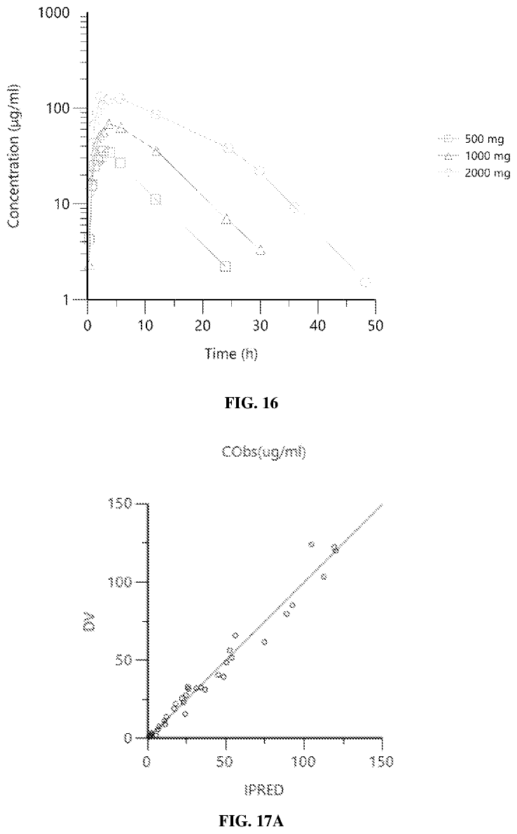

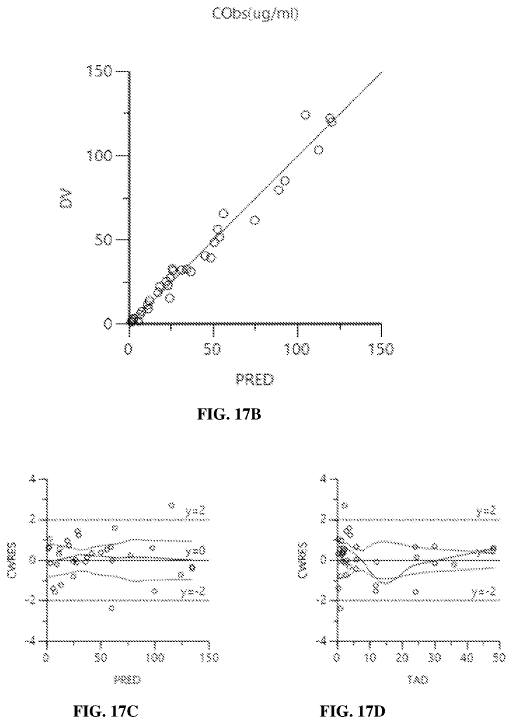

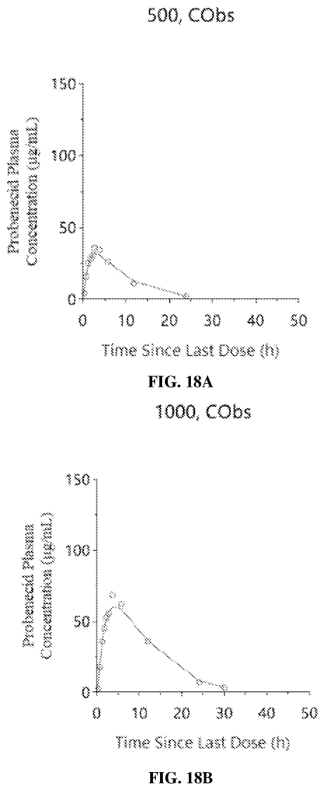

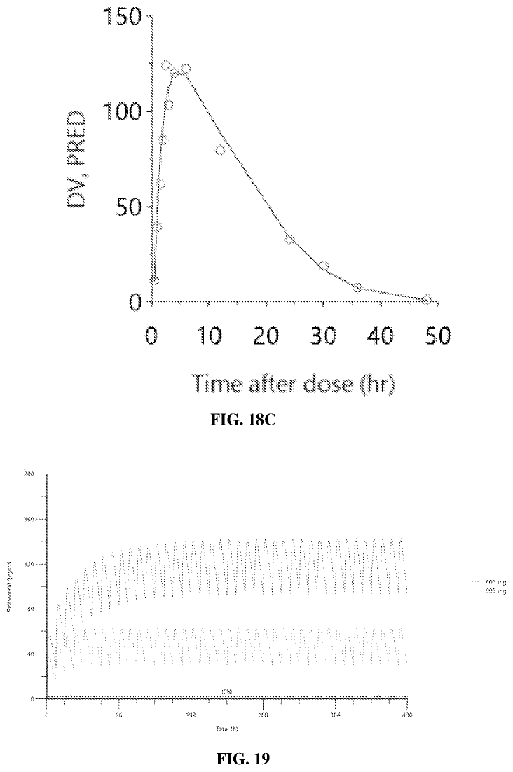

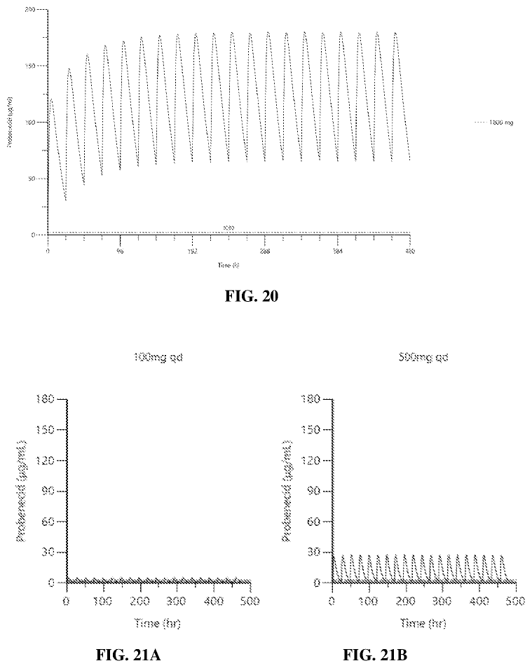

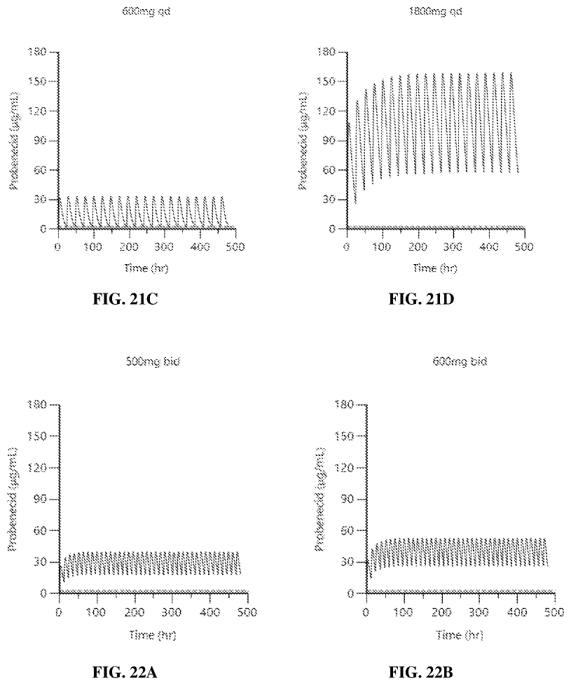

Mean plasma levels of probenecid after three oral dose administrations were obtained by digitization from a graph published by Selen et al., J. Pharm. Sci. 71:1238-1242 (1982) doi: 10.1002/jps.2600711114. A dose-stratified concentration-time plot of the digitized data is shown in FIG. 16. A final PopPK model was developed using these concentration-time data and initial estimates from the manuscript. As illustrated in FIGS. 17A-17D, the final PopPK had a one-compartment structure with saturable elimination and first-order absorption. Diagnostic plots are shown for the final model in FIGS. 18A-18C, and indicate that the available data were well-described by the final model. The final popPK model was used to simulate exposures resulting from various dosing regimens of potential clinical interest. For 600 mg BID arm, the steady state was achieved at around 96 h, while it took about 192 h for 900 mg BID and 1800 mg QD arms (FIGS. 19-20). The steady state concentration after different dosing scenarios and its comparison with reported IC90 level are tabulated in Table 9. The C12, C24 and Cmax after single dose, ratio of free probenecid concentration at these time points with IC90 probenecid level and time to achieve IC90 level after different doses are listed in Table 10 seen below.

TABLE 9
Steady state concentration after different
probenecid doses and IC90 ratio.

| | | | C_{trough} | |
|---|---|---|---|---|
| | | | concentration | |
| | | | (μmol/L) with | C_{trough} |
| | | C_{trough} | 95% protein | concentration |
| Dose (mg) | Frequency | (μg/ml) | binding | Ratio with IC₉₀ |
| 100 | QD | 0.2 | 0.04 | 0.1 |
| 500 | BID | 18.12 | 3.17 | 8.71 |
| 500 | QD | 1.77 | 0.31 | 0.85 |
| 600 | BID | 26.54 | 4.65 | 12.76 |
| 600 | QD | 2.47 | 0.43 | 1.19 |
| 900 | BID | 83.57 | 14.64 | 40.18 |
| 1000 | BID | 129.96 | 22.77 | 62.48 |
| 1800 | QD | 57.38 | 10.05 | 27.59 |

TABLE 10
Probenecid concentration after single doses and time taken to reach IC90 level.

| | | | | | | | | Time to |
|---|---|---|---|---|---|---|---|---|
| | | | | | | | | reach |
| | | C12 | C24 | Cmax | Free | Free | Free | IC90 |
| Dose (mg) | | (μg/ml) | (μg/ml) | (μg/ml) | C12/IC90 | C24/IC90 | Cmax/IC90 | level (h) |
| 100 | QD | 1.53 | 0.19 | 4.83 | 0.74 | 0.09 | 2.32 | 0.9 |
| 500 | BID | 11.13 | 15.17 | 34.27 | 5.35 | 7.29 | 16.48 | 0.472 |
| 500 | QD | 11.13 | 1.66 | 26.11 | 5.35 | 0.8 | 12.55 | 0.472 |
| 600 | BID | 14.46 | 20.49 | 42.73 | 6.95 | 9.85 | 20.54 | 0.457 |
| 600 | QD | 14.46 | 2.29 | 31.85 | 6.95 | 1.1 | 15.31 | 0.457 |
| 900 | BID | 26.53 | 41.54 | 71.25 | 12.75 | 19.97 | 34.25 | 0.433 |
| 1000 | BID | 31.17 | 50.12 | 81.66 | 14.99 | 24.1 | 39.26 | 0.429 |
| 1800 | QD | 76.06 | 26.37 | 107.87 | 36.57 | 12.68 | 51.86 | 0.409 |

In the simulation study, probenecid data from healthy adult male subjects were used to describe drug absorption and disposition. Covariate analysis could not be performed due to small sample size. Final population PK model, obtained using this data and initial estimates from a saturation kinetics model, was used to simulate various dosing scenarios of potential interest. Specifically, doses of 100 mg QD, 500 mg QD, 600 mg QD, 1800 mg QD, 600 mg BID, 600 mg BID, 900 mg BID, and 1000 mg BID were selected for further analysis. Steady state concentration and comparison with IC90 levels were estimated. Additionally, concentrations at 12 and 24 h after single dose administration and maximum concentration, comparison with reported IC90 level and time to reach in plasma following oral administration were estimated. These simulations show that these dosing regimens would lead to achieving concentration multifold higher than drug level required for 90% inhibition of viral replication. Overall, simulation results indicate that all dosing regimens except for 100 mg QD and 500 mg QD resulted in higher than IC90 probenecid exposure in plasma even with 95% protein binding. These simulations show that at steady state 500 mg bid, 600 mg bid, 900 mg bid, 1000 mg bid and 1800 mg qd dosing regimens would lead to achieving concentration multifold higher than drug level required for 90% inhibition of viral replication.

Using the same dose levels and associated plasma concentrations described above and the IC50 for A/WSN/33 (H1N1) (0.5 nM) described in Perwitasari, et al., Antimicrob. Agents Chemother, 57:475-483 (2013). doi: 10.1128/AAC.01532-12), similar tables of an exposure response relationship can be created. These data demonstrate that plasma concentrations approximately 280-150,000-fold higher than the protein binding adjusted IC50 value can be achieved at all dose levels for probenecid at steady state. Additionally, the data demonstrate that following a single dose, the plasma concentrations remain greater than 1,000-350,000 fold higher than the protein binding adjusted IC50 value 24 hours after dosing.

TABLE 11
Steady state concentration after different probenecid
doses and IC50 of 0.5 nM using Influenza disease model.

| | | | Ctrough | |
|---|---|---|---|---|
| | | | concentration | |
| | | | (μmol/L) | Ctrough |
| | | | with 95% | concentration |
| | | Ctrough | protein | Ratio with |
| Dose (mg) | Frequency | (μg/ml) | binding | IC50 |
| 100 | QD | 0.2 | 0.04 | 280 |
| 500 | BID | 18.12 | 3.17 | 22218 |
| 500 | QD | 1.77 | 0.31 | 2173 |
| 600 | BID | 26.54 | 4.65 | 32590 |
| 600 | QD | 2.47 | 0.43 | 3014 |
| 900 | BID | 83.57 | 14.64 | 102607 |
| 1000 | BID | 129.96 | 22.77 | 159588 |
| 1800 | QD | 57.38 | 10.05 | 70437 |

IC50 = 0.5 nM or 0.000143 μg/mL

TABLE 12
Probenecid concentration after single doses and time taken
to reach IC50 level using Influenza disease model.

| | | | | | | | | Time to |
|---|---|---|---|---|---|---|---|---|
| | | | | | | | | reach |
| Dose | | C12 | C24 | Cmax | Free | Free | Free | IC50 |
| (mg) | | (μg/ml) | (μg/ml) | (μg/ml) | C12/IC50 | C24/IC50 | Cmax/IC50 | level (h) |
| 100 | QD | 1.53 | 0.19 | 4.83 | 10699 | 1329 | 33776 | 0.9 |
| 500 | BID | 11.13 | 15.17 | 34.27 | 77832 | 106084 | 239650 | 0.472 |
| 500 | QD | 11.13 | 1.66 | 26.11 | 77832 | 11608 | 182587 | 0.472 |
| 600 | BID | 14.46 | 20.49 | 42.73 | 101119 | 143287 | 298811 | 0.457 |
| 600 | QD | 14.46 | 2.29 | 31.85 | 101119 | 16014 | 222727 | 0.457 |
| 900 | BID | 26.53 | 41.54 | 71.25 | 185524 | 290490 | 498252 | 0.433 |
| 1000 | BID | 31.17 | 50.12 | 81.66 | 217972 | 350490 | 571049 | 0.429 |
| 1800 | QD | 76.06 | 26.37 | 107.87 | 531888 | 184406 | 754336 | 0.409 |

IC50 = 0.000143 μg/mL

Simulations were also conducted in pediatric subjects. Allometric scaling based on body weights used as an empirical approach to predict concentrations in pediatric subjects. 10 mg/kg qd, 10 mg/kg bid, 20 mg/kg qd, 20 mg/kg bid dose was simulated. IC50 from preliminary virology data is 1 nM.

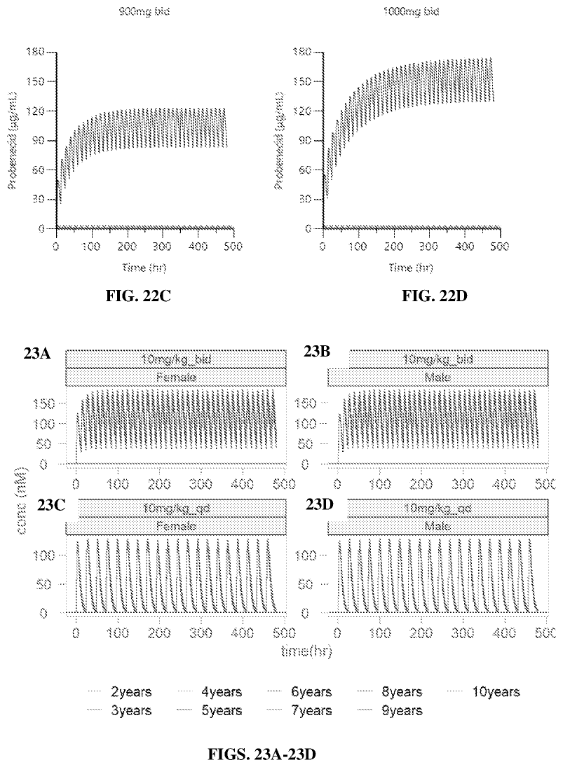

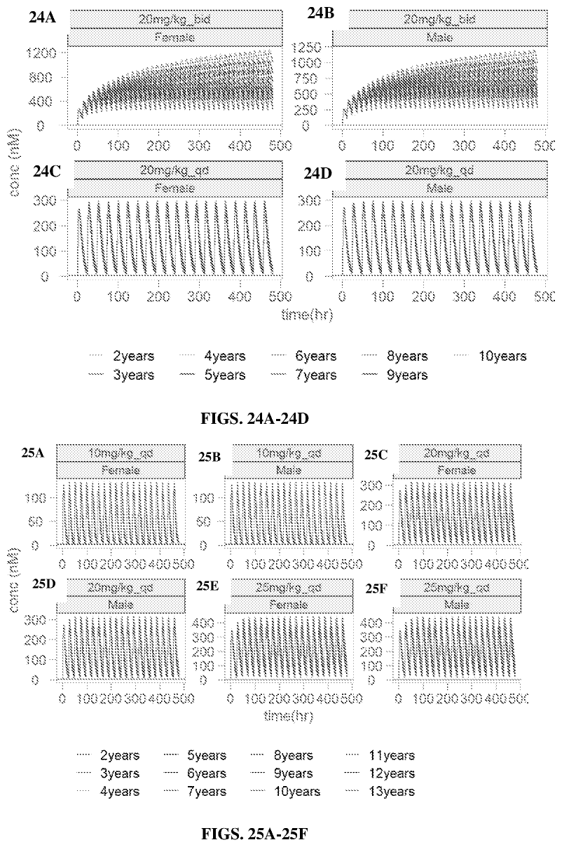

The results are shown in FIGS. 22A-25F.

### Example 5: Probenecid Prophylactically and Therapeutically Reduces Human Metapneumovirus (HMPV) Infection, but not Bacteria or Ad5

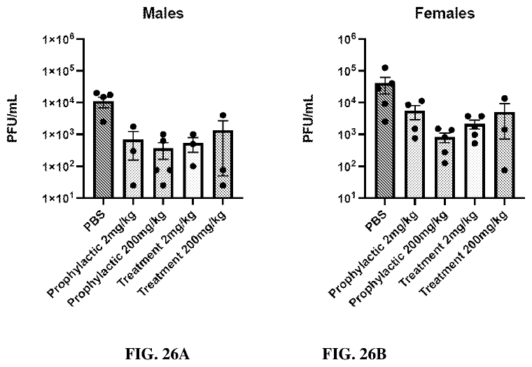

BALB/c mice were prophylactically (24 h prior to) or post-infection (24 h post) (“treatment”) treated with probenecid at the indicated concentrations. Mice (n=5/sex/group) were challenged i.n. with 1e6 PFU hMPV CAN83 (A2 strain). Lungs were collected 5 dpi and a plaque assay performed. The results are illustrated in FIGS. 26A-26B.

In other experiments, probenecid did not kill bacteria (neither free E. coli bacteria nor bacterial replication in macrophage of macrophage-associated bacteria Burkholderia) presumably because it is not cytotoxic. Additionally, probenecid did not statistically significantly reduce infection of A549 cells infected with the double-stranded DNA adenovirus serotype 5 (Ad5). However, only limited condition were tested and early effects, e.g., 24 and 8 hours post infection were not examined.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A compound having the structure of: 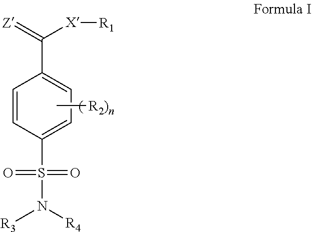
wherein:
(a) Z′ is O, NR₅, or S;
(b) X′ is absent, O, NR₅, or S;
(c) R₁ is hydrogen, a substituted or unsubstituted alkyl, a substituted or unsubstituted alkenyl, a substituted or unsubstituted alkynyl, a substituted or unsubstituted aryl, a substituted or unsubstituted heteroaryl, a substituted or unsubstituted polyaryl, a substituted or unsubstituted polyheteroaryl, an azo, an alkoxy, a polyether, a thiol, a sulfanimine, an amino, a carbonate, an ester, an amide, a carbamate, an imine, a substituted or unsubstituted carbonyl, a hydroxyl, a polyol, a phosphonyl, sulfinyl, a sulfonamide, a nitro, a cyano, a lipid, a peptide, a cholesterol, a phytosterol, a glycoside, or a glucuronide;
(d) n is an integer from 0 to 4;
(e) each R₂ is independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted aralkyl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen;
(f) R₃-R₅ are independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol; and
(g) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl,
with the proviso that the compound is not probenecid having the structure of:

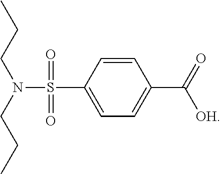 [SMILES: CCCN(CCC)S(=O)(=O)c1ccc(C(=O)O)cc1]

2. The compound of claim 1, wherein R₁ is hydrogen, a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, a substituted or unsubstituted heteropolyaryl, a polyol, a polyalkylene glycol, a lipid, a peptide, a cholesterol, a phytosterol, a glucuronide 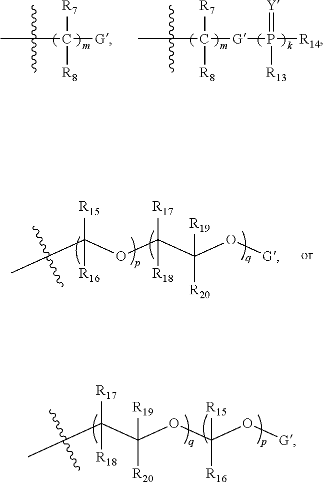
wherein G′ is hydrogen, a lipid, a peptide, a cholesterol, a phytosterol, a glycoside, a glucuronide, 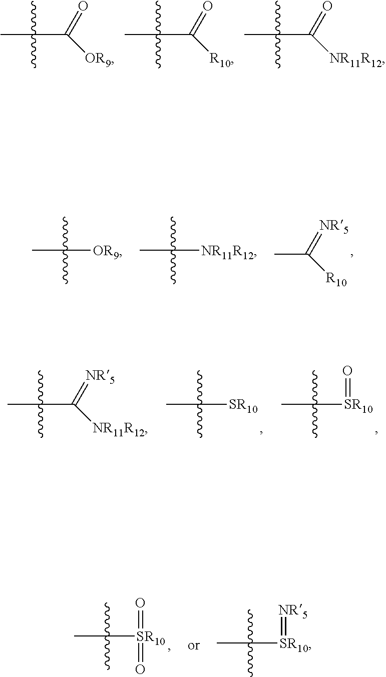
R₉-R₁₂ are independently hydrogen, a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, an alkoxy, a di-alkyl amino, or a halogen;
R′₅ is independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol, such as a hydrogen or a substituted or unsubstituted C₁-C₆ alkyl (e.g., an unsubstituted C₁-C₆ linear or branched alkyl, an unsubstituted C₁-C₆ cycloalkyl, an unsubstituted C₁-C₄ linear or branched alkyl, an unsubstituted C₁-C₄ cycloalkyl, an unsubstituted C₁-C₃ linear or branched alkyl, an unsubstituted C₁-C₃ cycloalkyl, etc.);
m, k, p, and q are independently an integer from 0 to 20, from 0 to 18, from 0 to 16, from 0 to 14, from 0 to 12, from 0 to 10, from 0 to 8, from 0 to 6, from 0 to 4, from 0 to 3, or from 0 to 2, such as 0 or 1;
each Y′ is independently O or S;
each occurrence of R₇, R₈, and R₁₅-R₂₀ is independently hydrogen, a substituted or unsubstituted C₁-C₂₀ alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen, or
R₇ and R₈ together, R₁₅ and R₁₆ together, and/or R₁₇ and R₁₈ together, with the carbon atom to which they are attached, form a C₁-C₂₀ cycloalkyl, or
when X′ is NR₅, m is not 0, at least one of p and q is not 0, then (i) R₇ is hydrogen and R₈ is a substituted or unsubstituted C₁-C₂₀ alkyl that form a ring together with R₅ that includes the adjoining N and C atoms, (ii) R₁₅ is hydrogen and R₁₆ is a substituted or unsubstituted C₁-C₂₀ alkyl that form a ring together with R₅ that includes the adjoining N and C atoms, and/or (iii) R₁₇ is hydrogen and R₁₈ is a substituted or unsubstituted C₁-C₂₀ alkyl that form a ring together with R₅ that includes the adjoining N and C atoms; and
R₁₃ and R₁₄ are independently hydrogen, a substituted or unsubstituted C₁-C₂₀ alkyl, or an alkoxy.

3. The compound of claim 2, wherein R₁ is an unsubstituted C₁-C₂₀ linear or branched alkyl, an unsubstituted C₃-C₂₀ cycloalkyl, a C₁-C₂₀ haloalkyl, an unsubstituted aryl, an unsubstituted polyaryl, an unsubstituted heteroaryl, an unsubstituted heteropolyaryl, a polyalkylene glycol, a lipid, a peptide, a cholesterol, a phytosterol, a glucuronide, 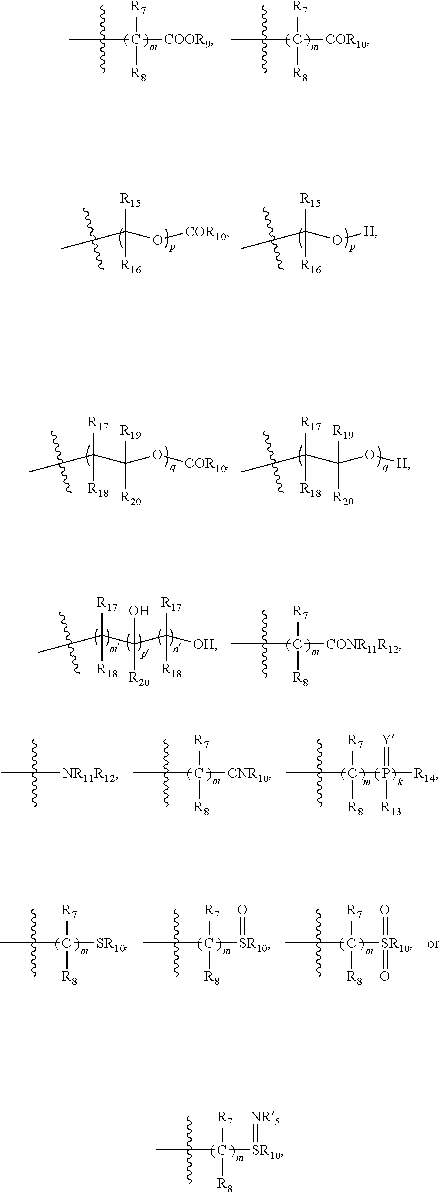
and wherein m, m′, p′, and n′ are independently an integer from 0 to 10, from 0 to 8, from 0 to 6, from 0 to 4, from 0 to 3, from 0 to 2, or 0 or 1, p is an integer from 1 to 6, from 1 to 4, from 1 to 3, or 1 or 2, and k is an integer from 1 to 6, from 1 to 4, or.

4. The compound of claim 1, wherein Z′ is O, X′ is absent or O, and R₁ is a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, a substituted or unsubstituted heteropolyaryl, a polyalkylene glycol, a lipid, a peptide, a cholesterol, a phytosterol, a glycoside, or a glucuronide.

5. The compound of claim 1, wherein Z′ is O or NR₅ and 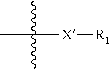
is 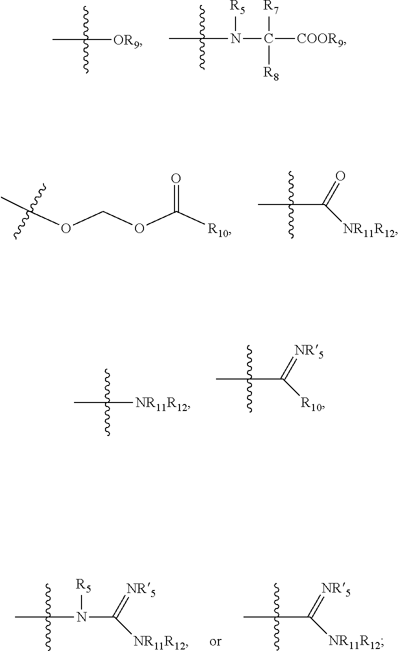
each occurrence of R₇ and R₈ is independently hydrogen, a substituted or unsubstituted C₁-C₂₀ alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen, or
R₇ and R₈ together, with the carbon atom to which they are attached, form a C₁-C₂₀ cycloalkyl;
R₉-R₁₂ are independently hydrogen, a substituted or unsubstituted C₁-C₂₀ linear or branched alkyl (e.g., haloalkyl), a substituted or unsubstituted C₃-C₂₀ cycloalkyl, a substituted or unsubstituted C₁-C₂₀ linear or branched heteroalkyl, a substituted or unsubstituted C₃-C₂₀ heterocyclyl, a substituted or unsubstituted aryl, a substituted or unsubstituted polyaryl, an alkoxy, a di-alkyl amino, or a halogen; and
R′₅ is independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol, such as a hydrogen or a substituted or unsubstituted C₁-C₆ alkyl (e.g., an unsubstituted C₁-C₆ linear or branched alkyl, an unsubstituted C₁-C₆ cycloalkyl, an unsubstituted C₁-C₄ linear or branched alkyl, an unsubstituted C₁-C₄ cycloalkyl, an unsubstituted C₁-C₃ linear or branched alkyl, an unsubstituted C₁-C₃ cycloalkyl, etc.).

6. The compound of claim 2, wherein Z′ is O, X′ is O, and R₁ is 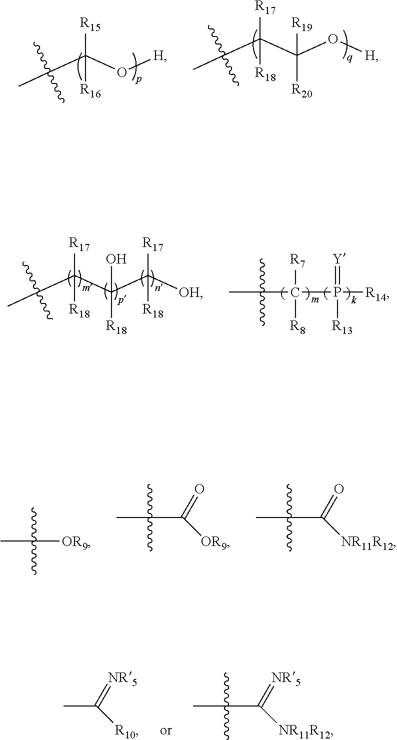
optionally wherein Z′ is O, X′ is O, and R₁ is 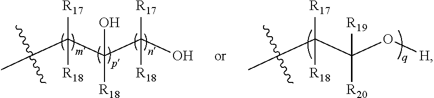
m′ and n′ are independently an integer from 0 to 6, from 0 to 5, from 0 to 4, from 0 to 3, from 1 to 3, such as 1 or 2; p′ is an integer from 1 to 10, from 1 to 8, from 1 to 6, from 1 to 4, or from 1 to 3; q is an integer from 1 to 10, from 1 to 8, from 1 to 6, from 1 to 4, or from 1 to 3; and each occurrence of R₁₇-R₂₀ are independently hydrogen, hydroxyl, —SH, —(CH₂)₁₋₆NR₂₂R₂₃, —(CH₂)₁₋₆OH, —(CH₂)₁₋₆SH, or an unsubstituted C₁-C₁₀ alkyl.

7. The compound of claim 2, wherein Z′ is O, X′ is S, and R₁ is

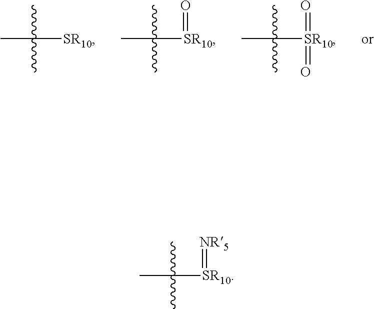

8. The compound of claim 2, wherein each occurrence of R₇, R₈, and R₁₅-R₂₀ is independently hydrogen, a substituted or unsubstituted C₁-C₂₀ alkyl, —(CH₂)₁₋₆NR₂₂R₂₃, —(CH₂)₁₋₆OH, a substituted or unsubstituted aralkyl, —(CH₂)₁₋₆SH, —(CH₂)₁₋₆S(O)₀₋₂CH₃, —(CH₂)₁₋₆NHC(═NH)NH₂, -(1H-indol-3-yl) methyl, -(1H-imidazol-4-yl)methyl, —(CH₂)₀₋₆COOR₂₁, —(CH₂)₀₋₆CONR₂₂R₂₃, a substituted or unsubstituted aryl, an aryl-C₁₋₃ alkyl, CH₂-indol-3-yl, —(CH₂)₁₋₆SCH₃, —CH₂-imidazol-4-yl, CH(OH)(CH₂)₀₋₅CH₃, —CH₂((4′-OH)-Ph), and R₂₁-R₂₃ are independently hydrogen or an unsubstituted C₁₋₆ alkyl; or
wherein R₃ and R₄ are independently hydrogen, a substituted or unsubstituted alkyl, a substituted or unsubstituted aryl, a substituted or unsubstituted hetercyclyl, a substituted or unsubstituted heteroaryl, an alkoxy, an amino, or an imine, optionally R₃ and R₄ are independently hydrogen or a substituted or unsubstituted C₁-C₂₀ alkyl such as an unsubstituted methyl, ethyl, propyl, butyl, pentyl, or hexyl, for example, an unsubstituted propyl; or
wherein each occurrence of R₂ is independently hydrogen, hydroxyl, —SH, —(CH₂)₁₋₆NR₂₂R₂₃, —(CH₂)₁₋₆OH, —(CH₂)₁₋₆SH, or an unsubstituted C₁-C₁₀ alkyl, such as hydrogen; or
wherein R₅ and/or R′₅ is(are) independently hydrogen or a substituted or unsubstituted C₁-C₂₀ alkyl; or
a combination thereof.

9.-11. (canceled)

12. The compound of claim 1, wherein the peptide comprises or is a peptide selected from RKKRRQRRR (SEQ ID NO:6), RRRRRRRR (SEQ ID NO:7), RKKRRRESRKKRRRES (SEQ ID NO:8), GRPRESGKKRKRKRLKP (SEQ ID NO:9), RQIKIWFQNRRMKWKK (SEQ ID NO:10), GRRRRRRRRRPPQ (SEQ ID NO:11), LLIILRRRIRKQAHAHSK (SEQ ID NO:12), RVRVFVVHIPRLT (SEQ ID NO:13), GALFLGFLGAAGSTMGAWSQPKKKRVK (SEQ ID NO:14), KLALKLALKALKAALKLA (SEQ ID NO:15), GWTLNSAGYLLGKINLKALAALAKKIL (SEQ ID NO:16), VSALK (SEQ ID NO:17), CSIPPEVKFNPFVYLI (SEQ ID NO:18), GIGAVLKVLTTGLPALISWIKRKRQQ (SEQ ID NO:19), HGLASTLTRWAHYNALIRAF (SEQ ID NO:20).

13. The compound of claim 1, wherein the compound is not any one of the compounds in Table 1, optionally wherein the compound is

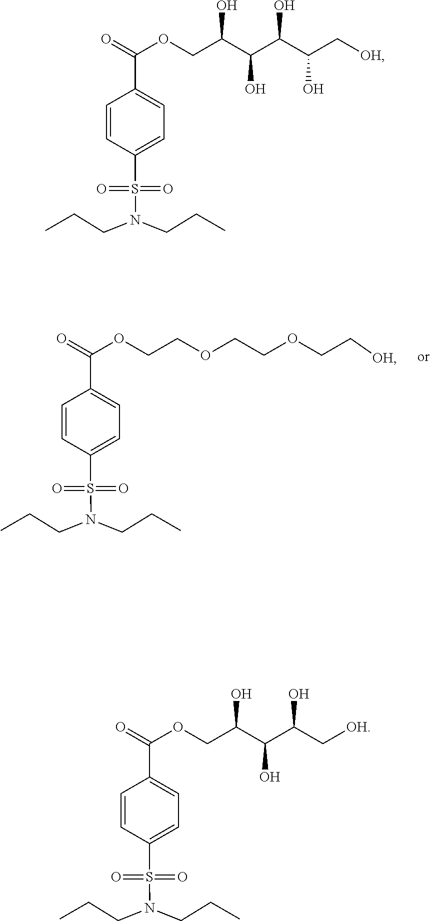 [SMILES: CCCN(CCC)S(=O)(=O)c1ccc(C(=O)OC[C@@H](O)[C@H](O)[C@@H](O)[C@@H](O)CO)cc1 ; CCCN(CCC)S(=O)(=O)c1ccc(C(=O)OCCOCCOCCO)cc1 ; "or" ; CCCN(CCC)S(=O)(=O)c1ccc(C(=O)OC[C@@H](O)[C@H](O)[C@@H](O)CO)cc1]

14. A pharmaceutical formulation comprising: one or more compounds of claim 1; and a pharmaceutically acceptable excipient and/or carrier, wherein the one or more compounds are in an effective amount to prevent, treat, or ameliorate one or more symptoms associated with a viral infection in a subject in need thereof.

15. The pharmaceutical formulation of claim 14, wherein the pharmaceutically acceptable carrier is nanoparticles, liposomes, cyclodextrins, or hydrogels, and optionally wherein the one or more prodrugs are encapsulated in, conjugated to, and/or complexed with the nanoparticles, liposomes, cyclodextrins, or hydrogels.

16. The pharmaceutical formulation of claim 14, wherein the pharmaceutical formulation is in the form of tablets, syrups, capsules, powders, or microneedles.

17. The pharmaceutical formulation of claim 14, further comprising one or more additional active agents, and optionally wherein the one or more additional active agents is/are one or more antiviral and/or anti-inflammatory agents.

18. A method of treating or preventing a viral infection in a subject comprising administering the subject an effective amount of a compound having the structure of: 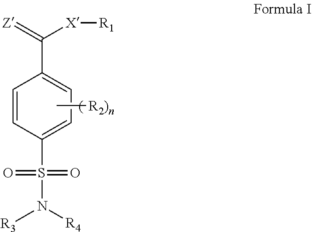
wherein:
(a) Z′ is O, NR₅, or S;
(b) X′ is absent, O, NR₅, or S;
(c) R₁ is hydrogen, a substituted or unsubstituted alkyl, a substituted or unsubstituted alkenyl, a substituted or unsubstituted alkynyl, a substituted or unsubstituted aryl, a substituted or unsubstituted heteroaryl, a substituted or unsubstituted polyaryl, a substituted or unsubstituted polyheteroaryl, an azo, an alkoxy, a polyether, a thiol, a sulfanimine, an amino, a carbonate, an ester, an amide, a carbamate, an imine, a substituted or unsubstituted carbonyl, a hydroxyl, a polyol, a phosphonyl, sulfinyl, a sulfonamide, a nitro, a cyano, a lipid, a peptide, a cholesterol, a phytosterol, a glycoside, or a glucuronide;
(d) n is an integer from 0 to 4;
(e) each R₂ is independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted aralkyl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen;
(f) R₃-R₅ are independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol; and
(g) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

19. (canceled)

20. The method of claim 18, wherein the subjects are infected with the virus, not infected with the virus, or a combination thereof;
optionally wherein the viral infection is due to DNA or RNA viruses; and
optionally wherein the viral infection is due to a DNA virus belonging to the family Adenoviridae, Papoviridae, Herpesviridae, Poxviridae, Anelloviridae or Pleolipoviridae; or an RNA virus belonging to the family Reoviridae, Picornaviridae, Caliciviridae, Togaviridae, Arenaviridae, Flaviviridae, Orthomyxoviridae, Paramyxoviridae, Bunyaviridae, Rhabdoviridae, Filoviridae, Coronaviridae, Astroviridae, Bornaviridae, Arteriviridae, Nymaviridae, Pneumoviridae, Flaviviridae, Hepeviridae/Nodaviridae, Picornaviridae, or Togaviridae.

21.-23. (canceled)

24. The method of claim 20, wherein the virus is a respiratory virus,
optionally wherein the virus is an influenza virus, such as influenza virus A, influenza virus B, or influenza virus C, respiratory syncytial virus (RSV), human metapneumovirus, coronaviruses, measles virus, parainfluenza virus, mumps virus, Zika virus, dengue virus, yellow fever virus, Japanese encephalitis virus, West Nile virus, Hepatitis A virus, Hepatitis B virus, or Hepatitis C virus; or
a coronavirus, such as a Severe acute respiratory syndrome-related coronavirus, a Bat Hp-betacoronavirus Zhejiang2013, a Rousettus bat coronavirus GCCDCl, a Rousettus bat coronavirus HKU9, a Eidolon bat coronavirus C704, a Pipistrellus bat coronavirus HKU5, a Tylonycteris bar coronovirus HKU4, a Middle East respiratory syndrome-related coronavirus, a Hedgehog coronavirus, a murine coronavirus, a Human coronavirus HKU1, a China Rattus coronavirus HKU24, a Betacoronavirus 1, a Myodes coronavirus 2JL14, a Human coronavirus NL63, a Human coronavirus 229E, and a Human coronavirus OC43.

25.-30. (canceled)

31. The method of claim 18, wherein the virus has an RNA genome optionally encoding an RNA-dependent RNA polymerase (RdRp), optionally is a member of the kingdom Orthornavirae, optionally utilizes a host organic anion transporter optionally selected from OAT1, OAT2, OAT3, OAT4, OAT5, OAT6, OAT7, rOAT8, OAT9, OAT10, and/or URAT1.

32. The method of claim 18, wherein the subject has one or more symptoms are selected from fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhea, tiredness, nausea, anosmia, skin rash, and combinations thereof, or
wherein the subject is asymptomatic.

33. (canceled)

34. The method of claim 18, wherein compound is in a delivery vehicle optionally selected from nanoparticles and liposomes; or
wherein the compound is in a pharmaceutical composition further comprising a pharmaceutically acceptable carrier and/or excipient.

35. (canceled)

36. The method of claim 18, wherein the compound is administered systemically, orally, parenterally, topically, or mucosally,
optionally wherein the compound is administered mucosally to the lungs, nasal mucosa, or combination thereof.

37.-38. (canceled)

39. The method of claim 18, wherein the compound is administered in an effective amount to reduce viral replication,
optionally wherein the compound is at a dosage of 10 mg-2,000 mg, or 600 mg, 900 mg, or 1,800 mg, optionally twice daily, optionally for 14 days, optionally for two weeks or more; or the compound is administered in a dose of 250 mg to 2,000 mg once or twice a day, optionally wherein the dose is 600 mg or 900 mg twice a day, or 1,800 mg once a day.

40.-44. (canceled)

45. The method of claim 18, wherein the subject or subjects is/are human(s), non-human mammal(s), or bird(s), optionally wherein the compound is formulated in the subject(s)'s drinking water, milk, or feed, and administered when the subject drinks the water or eats the feed.

46. (canceled)

47. The method of claim 45, wherein the subject(s) is/are chicken(s), optionally the virus is influenza A H5N1, or
wherein the subject(s) is/are pig(s), optionally the virus is influenza A H1N1; or
wherein the subject or subjects is/are human, optionally the virus is measles; or
wherein the subject or subjects is/are pediatric subjects, optionally between the ages of 2-10 inclusive.

48.-50. (canceled)

51. An animal feed comprising an effective amount of a compound selected from probenecid, or a metabolite or analog, or prodrug thereof, or pharmaceutically acceptable salt thereof, optionally wherein the compound has the structure of: 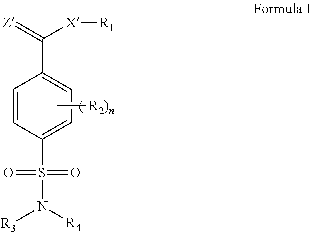
wherein:
(a) Z′ is O, NR₅, or S;
(b) X′ is absent, O, NR₅, or S;
(c) R₁ is hydrogen, a substituted or unsubstituted alkyl, a substituted or unsubstituted alkenyl, a substituted or unsubstituted alkynyl, a substituted or unsubstituted aryl, a substituted or unsubstituted heteroaryl, a substituted or unsubstituted polyaryl, a substituted or unsubstituted polyheteroaryl, an azo, an alkoxy, a polyether, a thiol, a sulfanimine, an amino, a carbonate, an ester, an amide, a carbamate, an imine, a substituted or unsubstituted carbonyl, a hydroxyl, a polyol, a phosphonyl, sulfinyl, a sulfonamide, a nitro, a cyano, a lipid, a peptide, a cholesterol, a phytosterol, a glycoside, or a glucuronide;
(d) n is an integer from 0 to 4;
(e) each R₂ is independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted aralkyl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen;
(f) R₃-R₅ are independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol; and
(g) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

52. (canceled)

53. A method of treating a subject for gout or hyperuricaemia, comprising administering a subject in need thereof an effective amount of a compound having the structure of: 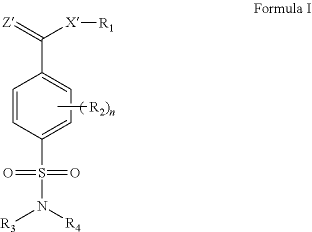
wherein:
(a) Z′ is O, NR₅, or S;
(b) X′ is absent, O, NR₅, or S;
(c) R₁ is hydrogen, a substituted or unsubstituted alkyl, a substituted or unsubstituted alkenyl, a substituted or unsubstituted alkynyl, a substituted or unsubstituted aryl, a substituted or unsubstituted heteroaryl, a substituted or unsubstituted polyaryl, a substituted or unsubstituted polyheteroaryl, an azo, an alkoxy, a polyether, a thiol, a sulfanimine, an amino, a carbonate, an ester, an amide, a carbamate, an imine, a substituted or unsubstituted carbonyl, a hydroxyl, a polyol, a phosphonyl, sulfinyl, a sulfonamide, a nitro, a cyano, a lipid, a peptide, a cholesterol, a phytosterol, a glycoside, or a glucuronide;
(d) n is an integer from 0 to 4;
(e) each R₂ is independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted heteropolyaryl, a substituted or unsubstituted aralkyl, a substituted or unsubstituted carbonyl, an alkoxy, an amido, an amino, a phosphonium, a phosphanyl, a phosphonyl, a silyl, a sulfinyl, a sulfonyl, a sulfate, a thiol, a hydroxyl, or a halogen;
(f) R₃-R₅ are independently a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, an alkoxy, an amido, an amino, an imine, or a thiol; and
(g) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, or a phosphonyl.

54. (canceled)


SEQUENCE LISTING (28 sequences)

SEQ ID NO: 1 - DNA, 29903 bp, Severe acute respiratory syndrome-related coronavirus
  source 1..29903: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 29882 bp, Severe acute respiratory syndrome-related coronavirus
  source 1..29882: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaa

SEQ ID NO: 3 - DNA, 29751 bp, Severe acute respiratory syndrome-related coronavirus
  source 1..29751: mol_type = other DNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggaactcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttagatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaa

SEQ ID NO: 4 - DNA, 30119 bp, Middle East respiratory syndrome-related coronavirus
  source 1..30119: mol_type = other DNA
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtatttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaattaacgaactattaatgagagttcaaagaccacccactctcttgttagtgttttcactctctcttttggtcactgcatcctcaaaacctctctatgtacctgagcattgtcagaattattctggttgcatgcttagggcttgtattaaaactgcccaagctgatacagctggtctttatacaaattttcgaattgacgtcccatctgcagaatcaactggtactcaatcagtttctgtcgatcttgagtcaacttcaactcatgatggtcctaccgaacatgttactagtgtgaatctttttgacgttggttactcagttaattaacgaactctatggattacgtgtctctgcttaatcaaatttggcagaagtaccttaactcaccgtatactacttgtttgtacatccctaaacccacagctaagtatacacctttagttggcacttcattgcaccctgtgctgtggaactgtcagctatcctttgctggttatactgaatctgctgttaattctacaaaagctttggccaaacaggacgcagctcagcgaatcgcttggttgctacataaggatggaggaatccctgatggatgttccctctacctccggcactcaagtttattcgcgcaaagcgaggaagaggagccattctccaactaagaaactgcgctacgttaagcgtagattttctcttctgcgccatgaagaccttagtgttattgtccaaccaacacactatgtcagggttacattttcagaccccaacatgtggtatctacgttcgggtcatcatttacactcagttcacaattggcttaaaccttatggcggccaacctgtttctgagtaccatattactctagctttgctaaatctcactgatgaagatttagctagagatttttcacccattgcgctctttttgcgcaatgtcagatttgagctacatgagttcgccttgctgcgcaaaactcttgttcttaatgcatcagagatctactgtgctaacatacatagatttaagcctgtgtatagagttaacacggcaatccctactattaaggattggcttctcgttcagggattttccctttaccatagtggcctccctttacatatgtcaatctctaaattgcatgcactggatgatgttactcgcaattacatcattacaatgccatgctttagaacttaccctcaacaaatgtttgttactcctttggccgtagatgttgtctccatacggtcttccaatcagggtaataaacaaattgttcattcttatcccattttacatcatccaggattttaacgaactatggctttctcggcgtctttatttaaacccgtccagctagtcccagtttctcctgcatttcatcgcattgagtctactgactctattgttttcacatacattcctgctagcggctatgtagctgctttagctgtcaatgtgtgtctcattcccctattattactgctacgtcaagatacttgtcgtcgcagcattatcagaactatggttctctatttccttgttctgtataactttttattagccattgtactagtcaatggtgtacattatccaactggaagttgcctgatagccttcttagttatcctcataatactttggtttgtagatagaattcgtttctgtctcatgctgaattcctacattccactgtttgacatgcgttcccactttattcgtgttagtacagtttcttctcatggtatggtccctgtaatacacaccaaaccattatttattagaaacttcgatcagcgttgcagctgttctcgttgtttttatttgcactcttccacttatatagagtgcacttatattagccgttttagtaagattagcctagtttctgtaactgacttctccttaaacggcaatgtttccactgttttcgtgcctgcaacgcgcgattcagttcctcttcacataatcgccccgagctcgcttatcgtttaagcagctctgcgctactatgggtcccgtgtagaggctaatccattagtctctctttggacatatggaaaacgaactatgttaccctttgtccaagaacgaatagggttgttcatagtaaactttttcatttttaccgtagtatgtgctataacactcttggtgtgtatggctttccttacggctactagattatgtgtgcaatgtatgacaggcttcaataccctgttagttcagcccgcattatacttgtataatactggacgttcagtctatgtaaaattccaggatagtaaaccccctctaccacctgacgagtgggtttaacgaactccttcataatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaa

SEQ ID NO: 5 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 9 aa, synthetic construct
RKKRRQRRR

SEQ ID NO: 7 - protein, 8 aa, synthetic construct
RRRRRRRR

SEQ ID NO: 8 - protein, 16 aa, synthetic construct
RKKRRRESRKKRRRES

SEQ ID NO: 9 - protein, 17 aa, synthetic construct
GRPRESGKKRKRKRLKP

SEQ ID NO: 10 - protein, 16 aa, synthetic construct
RQIKIWFQNRRMKWKK

SEQ ID NO: 11 - protein, 13 aa, synthetic construct
GRRRRRRRRRPPQ

SEQ ID NO: 12 - protein, 18 aa, synthetic construct
LLIILRRRIRKQAHAHSK

SEQ ID NO: 13 - protein, 13 aa, synthetic construct
RVRVFVVHIPRLT

SEQ ID NO: 14 - protein, 27 aa, synthetic construct
GALFLGFLGAAGSTMGAWSQPKKKRVK

SEQ ID NO: 15 - protein, 18 aa, synthetic construct
KLALKLALKALKAALKLA

SEQ ID NO: 16 - protein, 27 aa, synthetic construct
GWTLNSAGYLLGKINLKALAALAKKIL

SEQ ID NO: 17 - protein, 5 aa, synthetic construct
VSALK

SEQ ID NO: 18 - protein, 16 aa, synthetic construct
CSIPPEVKFNPFVYLI

SEQ ID NO: 19 - protein, 26 aa, synthetic construct
GIGAVLKVLTTGLPALISWIKRKRQQ

SEQ ID NO: 20 - protein, 20 aa, synthetic construct
HGLASTLTRWAHYNALIRAF

SEQ ID NO: 21 - DNA, 21 bp, synthetic construct
  source 1..21: mol_type = other DNA
tgcaaatgaatgcgaatgagg

SEQ ID NO: 22 - DNA, 21 bp, synthetic construct
  source 1..21: mol_type = other DNA
catgtacgttgctatccaggc

SEQ ID NO: 23 - DNA, 22 bp, synthetic construct
  source 1..22: mol_type = other DNA
catactcactcctgcactcatc

SEQ ID NO: 24 - DNA, 21 bp, synthetic construct
  source 1..21: mol_type = other DNA
cagccttccttcttgggtatg

SEQ ID NO: 25 - DNA, 20 bp, synthetic construct
  source 1..20: mol_type = other DNA
cggtcgtcgcataacacata

SEQ ID NO: 26 - DNA, 21 bp, synthetic construct
  source 1..21: mol_type = other DNA
ctccttaatgtcacgcacgat

SEQ ID NO: 27 - DNA, 21 bp, synthetic construct
  source 1..21: mol_type = other DNA
ccagggaatctcaaagggaaa

SEQ ID NO: 28 - DNA, 22 bp, synthetic construct
  source 1..22: mol_type = other DNA
ggcatagaggtctttacggatg